(19) United States
(12) Patent Application Publication
(10) Pub. No.: US 2015/0246130 A1
(43) Pub. Date: Sep. 3, 2015

(54) ANTIBODY FORMULATION

(71) Applicant: MEDIMMUNE, LLC, GAITHERSBURG, MD (US)

(72) Inventors: Steven BISHOP, Frederick, MD (US); Jenny S. WOLFORD, Brookeville, MD (US)

(21) Appl. No.: 14/506,224

(22) Filed: Oct. 3, 2014

Related U.S. Application Data
(63) Continuation of application No. 13/123,300, filed on Jun. 30, 2011, now abandoned.
(60) Provisional application No. 61/104,174, filed on Oct. 9, 2008.
(60) Provisional application No. 61/106,281, filed on Oct. 17, 2008.

(51) Int. Cl. A61K 47/26; A61K 47/22; A61K 39/395; C07K 16/24

(52) U.S. Cl. CPC A61K 47/26; C07K 16/249; A61K 47/22; A61K 39/3955; C07K 2317/76; A61K 2039/505

(57) ABSTRACT

The present invention provides high concentration liquid formulations of antibodies or fragments thereof that specifically bind to a human interferon alpha polypeptide The formulation provides for an anti-13H5 anti-human interferon alpha antibody A pre-filled syringe containing the formulation is also disclosed.

### 1. INTRODUCTION

The present invention relates to high concentration liquid formulations of antibodies or fragments thereof that specifically bind to a human interferon alpha polypeptide, which formulations exhibit stability, low to undetectable levels of antibody fragmentation, low to undetectable levels of aggregation, and very little to no loss of the biological activities of the antibodies, even during long periods of storage. The present invention also relates to methods of preventing, treating, managing or ameliorating symptoms associated with an interferon alpha mediated disease or disorder (for example, but not limited to, systemic lupus erythematosus, multiple sclerosis, inflammatory bowel disease, insulin dependent diabetes mellitus, psoriasis, autoimmune thyroiditis, rheumatoid arthritis and glomerulonephritis, transplant rejection, graft versus host disease) utilizing high concentration liquid formulations of antibodies or fragments thereof that specifically bind to a human interferon alpha polypeptide.

### 2. BACKGROUND

Type I interferons (IFN) (IFN-α, IFN-β, IFN-ω, IFN-τ) are a family of structurally related cytokines having antiviral, antitumor and immunomodulatory effects (Hardy et al. (2001) Blood 97:473; Cutrone and Langer (2001) J Biol. Chem. 276:17140). The human IFNα locus includes two subfamilies. The first subfamily consists of at least 14 non allelic genes and 4 pseudogenes having at least 75% homology. The second subfamily, αII or omega (ω), contains 5 pseudogenes and 1 functional gene which exhibits 70% homology with the IFNα genes. The subtypes of IFNα have different specific activities but they possess the same biological spectrum (Streuli et al. (1981) Proc. Natl. Acad. Sci. USA 78:2848) and have the same cellular receptor (Agnet M. et al. (1983) in “Interferon 5” Ed. I. Gresser p. 1-22, Academic Press, London).

Results from a number of groups suggest that IFN-α may enhance the maturation or activation of dendritic cells (DCs) (Santini, et al. (2000) J. Exp. Med. 191:1777; Luft et al. (1998) J. Immunol. 161:1947; Luft et al. (2002) Int. Immunol. 14:367; Radvanyi et al. (1999) Scand. J. Immunol. 50:499; Paquette et al. (1998) J. Leukoc. Biol. 64:358). Furthermore, increased expression of type I interferons has been described in numerous autoimmune diseases (Foulis et al. (1987) Lancet 2:1423; Hooks et al. (1982) Arthritis Rheum 25:396; Hertzog et al. (1988) Clin. Immunol. Immunopathol. 48:192; Hopkins and Meager (1988) Clin. Exp. Immunol. 73:88; Arvin and Miller (1984) Arthritis Rheum. 27:582). The most studied examples of this are insulin-dependent diabetes mellitus (IDDM) (Foulis (1987) supra), systemic lupus erythematosus (SLE) (Hooks (1982) supra; Blanco et al. (2001) Science 294:1540; Ytterberg and Schnitzer (1982) Arthritis Rheum. 25:401; Batteux et al. (1999) Eur. Cytokine Netw. :509), and autoimmune thyroiditis (Prummel and Laurberg (2003) Thyroid 13:547; Mazziotti et al. (2002) J. Endocrinol. Invest. 25:624; You et al. (1999) Chin. Med. J. 112:61; Koh et al. (1997) Thyroid 7:891), which are all associated with elevated levels of IFN α, and rheumatoid arthritis (RA) (Hertzog (1988), Hopkins and Meager (1988), Arvin and Miller (1984), supra) in which IFN-β may play a more significant role.

Moreover, administration of interferon α has been reported to exacerbate underlying disease in patients with psoriasis, autoimmune thyroiditis and multiple sclerosis and to induce an SLE like syndrome in patients without a previous history of autoimmune disease. Interferon α has also been shown to induce glomerulonephritis in normal mice and to accelerate the onset of the spontaneous autoimmune disease of NZB/W mice. Further, IFN-α therapy has been shown in some cases to lead to undesired side effects, including fever and neurological disorders. Hence, there are pathological situations in which inhibition of IFN-α activity may be beneficial to the patient and a need exists for therapeutic agents (e.g., anti-interferon alpha antibody formulations) effective in inhibiting IFN-α activity.

Currently, many antibodies are provided as lyophilized formulations. Lyophilized formulations of antibodies have a number of limitations, including a prolonged process for lyophilization and resulting high cost for manufacturing. In addition, a lyophilized formulation has to be reconstituted aseptically and accurately by healthcare practitioners prior to administering to patients. The reconstitution step itself requires certain specific procedures, for example: (1) a sterile diluent (i.e., water for intravenous administration and 5% dextrose in water for intramuscular administration) is added to the vial containing lyophilized antibody, slowly and aseptically, and the vial must be swirled very gently for 30 seconds to avoid foaming; (2) the reconstituted antibody may need to stand at room temperature for a minimum of 20 minutes until the solution clarifies; and (3) the reconstituted preparation must be administered within six (6) hours after the reconstitution. Such reconstitution procedure is cumbersome and the time limitation after the reconstitution can cause a great inconvenience in administering the formulation to patients, leading to significant waste, if not reconstituted properly, or if the reconstituted dose is not used within six (6) hours and must be discarded.

Thus, a need exists for liquid formulations of antibodies, in particular, anti-human interferon alpha antibodies, at a concentration comparable to or higher than the reconstituted lyophilized formulations so that there is no need to reconstitute the formulation prior to administration. This allows healthcare practitioners much quicker and easier administration of antibodies to a patient.

Prior liquid antibody preparations have short shelf lives and may lose biological activity of the antibodies resulting from chemical and physical instabilities during the storage. Chemical instability may be caused by deamidation, racemization, hydrolysis, oxidation, beta elimination or disulfide exchange, and physical instability may be caused by antibody denaturation, aggregation, precipitation or adsorption. Among those, aggregation, deamidation and oxidation are known to be the most common causes of the antibody degradation (Wang et al., 1988, J. of Parenteral Science & Technology 42(Suppl):S4-S26; Cleland et al., 1993, Critical Reviews in Therapeutic Drug Carrier Systems 10(4):307-377). Thus, there is a need for a stable liquid formulation of antibodies, in particular, stable liquid anti-human interferon alpha antibodies.

### 3. SUMMARY

The present invention relates to sterile, stable aqueous formulations comprising an antibody or fragment thereof that specifically binds human interferon alpha.

The present invention provides methods of stabilizing an anti-human interferon alpha antibody or fragment thereof.

The present invention further relates to processes of making a sterile, stable aqueous formulation comprising an antibody or fragment thereof that specifically binds human interferon alpha.

The present invention also encompasses methods of preventing, managing, treating or ameliorating an inflammatory disease or disorder, an autoimmune disease or disorder, a proliferative disease, an infection, a disease or disorder associated with or characterized by aberrant expression and/or activity of an interferon alpha polypeptide, a disease or disorder associated with or characterized by aberrant expression and/or activity of the interferon alpha receptor or one or more subunits thereof, or one or more symptoms thereof, said methods comprising administering to a subject in need thereof a prophylactically or therapeutically effective amount of an anti-human interferon alpha antibody formulation.

### 3.1. Definitions

All formulations of antibodies and/or antibody fragments that specifically bind to an antigen of interest (e.g., an interferon alpha polypeptide) are herein collectively referred to as “formulations of the invention”, “liquid formulations of the invention”, “high concentration stable liquid formulations of the invention”, “antibody liquid formulations of the invention”, or “antibody formulations of the invention”.

The terms “interferon alpha” and “IFN alpha” are used interchangeably and intended to refer to IFN alpha proteins encoded by a functional gene of the interferon alpha gene locus with 75% or greater sequence identity to IFN alpha 1 (GenBank accession number NP_{—}076918 or protein encoded by GenBank accession number NM 024013). Examples of IFN alpha subtypes include IFN alpha 1, alpha 2a, alpha 2b, alpha 4, alpha 5, alpha 6, alpha 7, alpha 8, alpha 10, alpha 13, alpha 14, alpha 16, alpha 17 and alpha 21. The term “interferon alpha” is intended to encompass recombinant forms of the various IFN alpha subtypes, as well as naturally occurring preparations that comprise IFN alpha proteins, such as leukocyte IFN and lymphoblastoid IFN. The term IFN alpha is not intended to encompass, for example, IFN omega alone, although a composition that comprises both IFN alpha and IFN omega is encompassed by the term IFN alpha.

The term “IFN alpha receptor” as used herein is intended to refer to members of the IFN alpha receptor family of molecules that are receptors for the ligand IFN alpha. Examples of IFN alpha receptors are IFN alpha receptor 1 (GenBank accession number NM_{—}000629 and NP_{—}000620) and IFN alpha receptor 2 (GenBank accession number NM_{—}207585 and NP_{—}997468).

As used herein, the term “subject” includes any human or nonhuman animal. The term “nonhuman animal” includes all vertebrates, for example, but not limited to, mammals and non-mammals, such as nonhuman primates, sheep, dogs, cats, horses, cows, chickens, amphibians, reptiles, etc.

The term “antibody” as referred to herein encompasses whole antibodies and any antigen binding fragment (i.e., “antigen-binding portion”) or single chains thereof. An “antibody” refers to a glycoprotein comprising at least two heavy (H) chains and two light (L) chains inter-connected by disulfide bonds, or an antigen binding portion thereof. Each heavy chain is comprised of a heavy chain variable region (abbreviated herein as V_{H}) and a heavy chain constant region. The heavy chain constant region is comprised of three domains, C_{H1}, C_{H2} and C_{H3}. Each light chain is comprised of a light chain variable region (abbreviated herein as V_{L}) and a light chain constant region. The light chain constant region is comprised of one domain, C_{L}. The V_{H} and V_{L} regions can be further subdivided into regions of hypervariability, termed complementarity determining regions (CDR), interspersed with regions that are more conserved, termed framework regions (FR). Each V_{H} and V_{L} is composed of three CDRs and four FRs, arranged from amino-terminus to carboxy-terminus in the following order: FR1, CDR1, FR2, CDR2, FR3, CDR3, FR4. The variable regions of the heavy and light chains contain a binding domain that interacts with an antigen. The constant regions of the antibodies may mediate the binding of the immunoglobulin to host tissues or factors, including various cells of the immune system (for example, but not limited to, effector cells) and the first component (Clq) of the classical complement system. Antibodies may be derived from any mammal, including, but not limited to, humans, monkeys, pigs, horses, rabbits, dogs, cats, mice, etc. The term “antibody” refers to monoclonal antibodies, multispecific antibodies, human antibodies, humanized antibodies, camelised antibodies, chimeric antibodies, single-chain Fvs (scFv), single chain antibodies, single domain antibodies, Fab fragments, F(ab′) fragments, disulfide-linked Fvs (sdFv), and anti-idiotypic (anti-Id) antibodies (including, for example, but not limited to, anti-Id antibodies to antibodies of the invention), intrabodies, and epitope-binding fragments of any of the above. Immunoglobulin molecules can be of any type (e.g., IgG, IgE, IgM, IgD, IgA and IgY), class (e.g., IgG₁, IgG₂, IgG₃, IgG₄, IgA₁ and IgA₂) or subclass.

The term “antigen-binding portion” of an antibody (or simply “antibody portion”), as used herein, refers to one or more fragments of an antibody that retain the ability to specifically bind to an antigen (e.g., IFN alpha). It has been shown that the antigen-binding function of an antibody can be performed by fragments of a full-length antibody. Examples of binding fragments encompassed within the term “antigen-binding portion” of an antibody include, but are not limited to, (i) a Fab fragment, a monovalent fragment consisting of the V_{L}, V_{H}, C_{L} and C_{H1} domains; (ii) a F(ab′)2 fragment, a bivalent fragment comprising two Fab fragments linked by a disulfide bridge at the hinge region; (iii) a Fd fragment consisting of the V_{H} and C_{H1} domains; (iv) a Fv fragment consisting of the V_{L} and V_{H} domains of a single arm of an antibody, (v) a dAb fragment (Ward et al., (1989) Nature 341:544-546), which consists of a V_{H} domain; and (vi) an isolated complementarity determining region (CDR). Furthermore, although the two domains of the Fv fragment, V_{L} and V_{H}, are coded for by separate genes, they can be joined, using recombinant methods, by a synthetic linker that enables them to be made as a single protein chain in which the V_{L} and V_{H} regions pair to form monovalent molecules (known as single chain Fv (scFv); see e.g., Bird et al. (1988) Science 242:423-426; and Huston et al. (1988) Proc. Natl. Acad. Sci. USA 85:5879-5883). Such single chain antibodies are also intended to be encompassed within the term “antigen-binding portion” of an antibody. These antibody fragments are obtained using conventional techniques known to those with skill in the art, and the fragments are screened for utility in the same manner as are intact antibodies.

The terms “monoclonal antibody” or “monoclonal antibody composition” as used herein refer to a preparation of antibody molecules of single molecular composition. A monoclonal antibody composition displays a single binding specificity and affinity for a particular epitope.

The term “human antibody”, as used herein, is intended to include antibodies having variable regions in which both the framework and CDR regions are derived from human germline immunoglobulin sequences. Furthermore, if the antibody contains a constant region, the constant region also is derived from human germline immunoglobulin sequences. The human antibodies of the invention may include amino acid residues not encoded by human germline immunoglobulin sequences (for example, but not limited to, mutations introduced by random or site-specific mutagenesis in vitro or by somatic mutation in vivo). However, the term “human antibody”, as used herein, is not intended to include antibodies in which CDR sequences derived from the germline of another mammalian species, such as a mouse, have been grafted onto human framework sequences.

The term “human monoclonal antibody” refers to antibodies displaying a single binding specificity which have variable regions in which both the framework and CDR regions are derived from human germline immunoglobulin sequences. In one embodiment, the human monoclonal antibodies are produced by a hybridoma which includes a B cell obtained from a transgenic nonhuman animal, for example, but not limited to, a transgenic mouse, having a genome comprising a human heavy chain transgene and a light chain transgene fused to an immortalized cell.

The term “recombinant human antibody”, as used herein, includes all human antibodies that are prepared, expressed, created or isolated by recombinant means, such as (a) antibodies isolated from an animal (for example, but not limited to, a mouse) that is transgenic or transchromosomal for human immunoglobulin genes or a hybridoma prepared therefrom (described further below), (b) antibodies isolated from a host cell transformed to express the human antibody, for example, but not limited to, from a transfectoma, (c) antibodies isolated from a recombinant, combinatorial human antibody library, and (d) antibodies prepared, expressed, created or isolated by any other means that involve splicing of human immunoglobulin gene sequences to other DNA sequences. Such recombinant human antibodies have variable regions in which the framework and CDR regions are derived from human germline immunoglobulin sequences. In certain embodiments, however, such recombinant human antibodies can be subjected to in vitro mutagenesis (or, when an animal transgenic for human Ig sequences is used, in vivo somatic mutagenesis) and thus the amino acid sequences of the V_{H} and V_{L} regions of the recombinant antibodies are sequences that, while derived from and related to human germline V_{H} and V_{L} sequences, may not naturally exist within the human antibody germline repertoire in vivo.

The term “isotype” refers to the classification of an antibody's heavy or light chain constant region. The constant domains of antibodies are not involved in binding to antigen, but exhibit various effector functions. Depending on the amino acid sequence of the heavy chain constant region, a given human antibody or immunoglobulin can be assigned to one of five major classes of immunoglobulins: IgA, IgD, IgE, IgG, and IgM. Several of these classes may be further divided into subclasses (isotypes), e.g., IgG1 (gamma 1), IgG2 (gamma 2), IgG3 (gamma 3), and IgG4 (gamma 4), and IgA1 and IgA2. The heavy chain constant regions that correspond to the different classes of immunoglobulins are called α, δ, ε, γ, and μ, respectively. The structures and three-dimensional configurations of different classes of immunoglobulins are well-known. Human light chain constant regions may be classified into two major classes, kappa and lambda

An “epitope” is a term well understood in the art and means any chemical moiety that exhibits specific binding to an antibody. An “antigen” is a moiety or molecule that contains an epitope, and, as such, also specifically binds to antibody.

“Affinity” of an antibody for an epitope to be used in the treatment(s) described herein is a term well understood in the art and means the extent, or strength, of binding of antibody to epitope. Affinity may be measured and/or expressed in a number of ways known in the art, including, but not limited to, equilibrium dissociation constant (KD or Kd), apparent equilibrium dissociation constant (KD′ or Kd′), and IC50 (amount needed to effect 50% inhibition in a competition assay). It is understood that, for purposes of this invention, an affinity is an average affinity for a given population of antibodies which bind to an epitope. Values of KD′ reported herein in terms of mg IgG per mL or mg/mL indicate mg Ig per mL of serum, although plasma can be used. When antibody affinity is used as a basis for administration of the treatment methods described herein, or selection for the treatment methods described herein, antibody affinity can be measured before and/or during treatment, and the values obtained can be used by a clinician in assessing whether a human patient is an appropriate candidate for treatment.

As used herein, the term “avidity” is a measure of the overall binding strength (i.e., both antibody arms) with which an antibody binds an antigen. Antibody avidity can be determined by measuring the dissociation of the antigen-antibody bond in antigen excess using any means known in the art, such as, but not limited to, by the modification of indirect fluorescent antibody as described by Gray et al., J. Virol. Meth., 44:11-24. (1993)

As used herein, “specific binding” refers to antibody binding to a predetermined antigen. Typically, the antibody binds with a dissociation constant (K_{D}) of 10⁻⁸ M or less, and binds to the predetermined antigen with a K_{D} that is at least two-fold less than its K_{D} for binding to a non-specific antigen (for example, but not limited to, BSA, casein) other than the predetermined antigen or a closely-related antigen. The phrases “an antibody recognizing an antigen“and” an antibody specific for an antigen” are used interchangeably herein with the term “an antibody which binds specifically to an antigen”.

The term “immune response” refers to the action of, for example, lymphocytes, antigen presenting cells, phagocytic cells, granulocytes, and soluble macromolecules produced by the above cells or the liver (including antibodies, cytokines, and complement) that results in selective damage to, destruction of, or elimination from the human body of invading pathogens, cells or tissues infected with pathogens, cancerous cells, or, in cases of autoimmunity or pathological inflammation, normal human cells or tissues.

As used herein, an antibody that “inhibits the biological activity” of an IFN alpha subtype is intended to refer to an antibody that inhibits the activity of that subtype by at least about 10%, at least about 20%, at least about 30%, at least about 40%, at least about 50%, at least about 60%, at least about 70% or at least about 80%, as compared to the level of activity in the absence of the antibody, for example using a functional assay such as those described in US Patent Publication 2007/0014724A1, such as the Daudi cell proliferation assay. Alternatively, an antibody that “inhibits the biological activity” of an IFN alpha subtype can refer to an antibody that inhibits the activity of that subtype with an EC₅₀ of less than 200 nM or less 100 nM or less, 50 nM or less and 10 nM or less.

The term “antibody half-life” as used herein means a pharmacokinetic property of an antibody that is a measure of the mean survival time of antibody molecules following their administration. Antibody half-life can be expressed as the time required to eliminate 50 percent of a known quantity of immunoglobulin from the patient's body or a specific compartment thereof, for example, as measured in serum or plasma, i.e., circulating half-life, or in other tissues. Half-life may vary from one immunoglobulin or class of immunoglobulin to another. In general, an increase in antibody half-life results in an increase in mean residence time (MRT) in circulation for the antibody administered.

The term “excipient” as used herein refers to an inert substance which is commonly used as a diluent, vehicle, preservative, binder or stabilizing agent for drugs which imparts a beneficial physical property to a formulation, such as increased protein stability, increased protein solubility, and decreased viscosity. Examples of excipients include, but are not limited to, proteins (for example, but not limited to, serum albumin), amino acids (for example, but not limited to, aspartic acid, glutamic acid, lysinc, argininc, glycine), surfactants (for example, but not limited to, SDS, Tween 20, Tween 80, polysorbate and nonionic surfactants), saccharides (for example, but not limited to, glucose, sucrose, maltose and trehalose), polyols (for example, but not limited to, mannitol and sorbitol), fatty acids and phospholipids (for example, but not limited to, alkyl sulfonates and caprylate). For additional information regarding excipients, see Remington's Pharmaceutical Sciences (by Joseph P. Remington, 18^{th} ed., Mack Publishing Co., Easton, Pa.), which is incorporated herein in its entirety.

The phrase “pharmaceutically acceptable” as used herein means approved by a regulatory agency of the Federal or a state government, or listed in the U.S. Pharmacopeia, European Pharmacopia or other generally recognized pharmacopeia for use in animals, and more particularly in humans.

The terms “stability” and “stable” as used herein in the context of a liquid formulation comprising an antibody (including antibody fragment thereof) that specifically binds to an antigen of interest (e.g., an interferon alpha polypeptide) refer to the resistance of the antibody (including antibody fragment thereof) in the formulation to aggregation, degradation or fragmentation under given manufacture, preparation, transportation and storage conditions. The “stable” formulations of the invention retain biological activity under given manufacture, preparation, transportation and storage conditions. The stability of said antibody (including antibody fragment thereof) can be assessed by degrees of aggregation, degradation or fragmentation, as measured by HPSEC, static light scattering (SLS), Fourier Transform Infrared Spectroscopy (FTIR), circular dichroism (CD), urea unfolding techniques, intrinsic tryptophan fluorescence, differential scanning calorimetry, and/or ANS binding techniques, compared to a reference formulation. For example, a reference formulation may be a reference standard frozen at −70° C. consisting of 10 mg/ml of an antibody (including antibody fragment thereof) (for example, but not limited to, 13H5, 13H7 or 7H9) in histidine, pH 6.0-6.5 that contains 8% trehalose and 0.02% polysorbate 80, which reference formulation regularly gives a single monomer peak (≧97% area) by HPSEC. The overall stability of a formulation comprising an antibody (including antibody fragment thereof) can be assessed by various immunological assays including, for example, ELISA and radioimmunoassay using isolated antigen molecules.

The phrase “low to undetectable levels of aggregation” as used herein refers to samples containing no more than about 5%, no more than about 4%, no more than about 3%, no more than about 2%, no more than about 1% and no more than about 0.5% aggregation by weight of protein as measured by high performance size exclusion chromatography (HPSEC) or static light scattering (SLS) techniques.

The term “low to undetectable levels of fragmentation” as used herein refers to samples containing equal to or more than about 80%, about 85%, about 90%, about 95%, about 98% or about 99% of the total protein, for example, in a single peak as determined by HPSEC, or in two peaks (e.g., heavy- and light-chains) (or as many peaks as there are subunits) by reduced Capillary Gel Electrophoresis (rCGE), representing the non-degraded antibody or a non-degraded fragment thereof, and containing no other single peaks having more than about 5%, more than about 4%, more than about 3%, more than about 2%, more than about 1%, or more than about 0.5% of the total protein in each. The term “reduced Capillary Gel Electrophoresis” as used herein refers to capillary gel electrophoresis under reducing conditions sufficient to reduce disulfide bonds in an antibody.

As used herein, the terms “disorder” and “disease” are used interchangeably to refer to a condition in a subject in which the subject differs from a healthy, unaffected subject. In particular, the term “autoimmune disease” is used interchangeably with the term “autoimmune disorder” to refer to a condition in a subject characterized by cellular, tissue and/or organ injury caused by an immunologic reaction of the subject to its own cells, tissues and/or organs. The term “inflammatory disease” is used interchangeably with the term “inflammatory disorder” to refer to a condition in a subject characterized by inflammation, for example, but not limited to, chronic inflammation. Autoimmune disorders may or may not be associated with inflammation. Moreover, inflammation may or may not be caused by an autoimmune disorder. Certain conditions may be characterized as more than one disorder. For example, certain conditions may be characterized as both autoimmune and inflammatory disorders.

The terms “therapies” and “therapy” can refer to any protocol(s), method(s), and/or agent(s) that can be used in the prevention, treatment and/or management of a disease or disorder.

By the terms “treat,” “treating” or “treatment of” (or grammatically equivalent terms) it is meant that the severity of the subject's condition is reduced or at least partially improved or ameliorated and/or that some alleviation, mitigation or decrease in at least one clinical symptom is achieved and/or there is an inhibition or delay in the progression of the condition and/or prevention or delay of the onset of a disease or illness. Thus, the terms “treat,” “treating” or “treatment of” (or grammatically equivalent terms) refer to both prophylactic and therapeutic treatment regimes.

As used herein, the terms “manage,” “managing,” and “management” refer to the beneficial effects that a subject derives from a therapy (e.g., a prophylactic or therapeutic agent), which does not result in a cure of the disease. In certain embodiments, a subject is administered one or more therapies (e.g., one or more prophylactic or therapeutic agents) to “manage” a disease so as to prevent the progression or worsening of the disease.

As used herein, the terms “prevent,” “preventing,” and “prevention” refer to the inhibition of the development or onset of disease or disorder, or the prevention of the recurrence, onset, or development of one or more symptoms of a disease or disorder in a subject resulting from the administration of a therapy (e.g., a prophylactic or therapeutic agent), or the administration of a combination of therapies (e.g., a combination of prophylactic or therapeutic agents).

As used herein, the terms “prophylactic agent” and “prophylactic agents” refer to any agent(s) which can be used in the prevention of the onset, recurrence or development of a disease or disorder. In certain embodiments, the term “prophylactic agent” refers to an antibody that specifically binds to an interferon alpha polypeptide. In certain other embodiments, the term “prophylactic agent” refers to an agent other than an antibody that specifically binds to an interferon alpha polypeptide. In certain embodiments, a prophylactic agent is an agent which is known to be useful to or has been or is currently being used to the prevent or impede the onset, development, progression and/or severity of a disease or disorder.

As used herein, the term “immunomodulatory agent” and variations thereof including, but not limited to, immunomodulatory agents, immunomodulants or immunomodulatory drugs, refer to an agent that modulates a host's immune system. In a specific embodiment, an immunomodulatory agent is an agent that shifts one aspect of a subject's immune response. In certain embodiments, an immunomodulatory agent is an agent that inhibits or reduces a subject's immune system (i.e., an immunosuppressant agent). In certain other embodiments, an immunomodulatory agent is an agent that activates or increases a subject's immune system (i.e., an immunostimulatory agent). In accordance with the invention, an immunomodulatory agent used in the combination therapies of the invention does not include an antibody of the invention. Immunomodulatory agents include, but are not limited to, small molecules, peptides, polypeptides, proteins, nucleic acids (for example, but not limited to, DNA and RNA nucleotides including, but not limited to, antisense nucleotide sequences, triple helices, RNAi, and nucleotide sequences encoding biologically active proteins, polypeptides or peptides), antibodies, synthetic or natural inorganic molecules, mimetic agents, and synthetic or natural organic molecules.

As used herein, a “sufficient amount” or “an amount sufficient to” achieve a particular result refers to an amount of an antibody or composition of the invention that is effective to produce a desired effect, which is optionally a therapeutic effect (i.e., by administration of a therapeutically effective amount).

A “therapeutically effective” amount as used herein is an amount that provides some improvement or benefit to the subject. Stated in another way, a “therapeutically cffcctive” amount is an amount that provides some alleviation, mitigation, and/or decrease in at least one clinical symptom. Clinical symptoms associated with the disorders that can be treated by the methods of the invention are well-known to those skilled in the art. Further, those skilled in the art will appreciate that the therapeutic effects need not be complete or curative, as long as some benefit is provided to the subject.

A “therapeutically effective dosage” of an anti-IFN alpha antibody of the invention results in a decrease in severity of at least one disease symptom, an increase in frequency and duration of disease symptom-free periods, or a prevention of impairment or disability due to the disease affliction. For example, in the case of systemic lupus erythematosus (SLE), a therapeutically effective dose prevents further deterioration of at least one physical symptom associated with SLE, such as, for example, pain or fatigue. A therapeutically effective dose also prevents or delays onset of SLE, such as may be desired when early or preliminary signs of the disease are present. Likewise it includes delaying chronic progression associated with SLE. Laboratory tests utilized in the diagnosis of SLE include chemistries (including the measurement of IFN alpha levels), hematology, serology and radiology. Accordingly, any clinical or biochemical assay that monitors any of the foregoing may be used to determine whether a particular treatment is a therapeutically effective dose for treating SLE. One of ordinary skill in the art would be able to determine such amounts based on such factors as the subject's size, the severity of the subject's symptoms, and the particular composition or route of administration selected.

As used herein, the terms “non-responsive” and refractory” describe patients treated with a currently available therapy (e.g., prophylactic or therapeutic agent) for a disease or disorder. Such patients likely suffer from severe, persistently active disease and require additional therapy to ameliorate the symptoms associated with the disorder.

Concentrations, amounts, cell counts, percentages and other numerical values may be presented herein in a range format. It is also to be understood that such range format is used merely for convenience and brevity and should be interpreted flexibly to include not only the numerical values explicitly recited as the limits of the range but also to include all the individual numerical values or sub-ranges encompassed within that range as if each numerical value and sub-range is explicitly recited.

### 4. BRIEF DESCRIPTION OF THE DRAWINGS

For the purpose of illustrating representative embodiments of the invention, drawings are provided herein.

FIG. 1. Flow diagram of formulation manufacturing process used to produce 100 g/L 13H5 in 25 mM histidine, 8% trehalose, 0.02% polysorbate 20, pH 6.0.

FIG. 2. Stability of 13H5 formulations (100 mg/ml, formulation E) at 40° C. The formulations contain antibody generated from the same cell line using different manufacturing processes. The percent (%) aggregate content of formulation determined by SEC at various time points is plotted.

FIG. 3. Stability of 13H5 formulations (100 mg/ml, formulation E) at 5° C. The formulations contain antibody generated from the same cell line using different manufacturing processes. The percent (%) aggregate content of formulation determined by SEC at various time points is plotted.

FIG. 4. Stability of 13H5 formulations (100 mg/ml) at 40° C. Chart displays the percent (%) aggregate content of formulation determined by SEC at various time points.

FIG. 5. Concentration dependence of 13H5 formulation stability at 40° C. Chart displays the percent (%) aggregate content of formulation determined by SEC at various time points.

FIG. 6. pH dependence of 13H5 formulation (100 mg/ml) stability at 40° C. Chart displays the percent (%) aggregate content of formulation determined by SEC at various time points.

FIG. 7. Stability of 13H5 (Formulation A, 100 mg/ml) is very similar to that of antibody X (Mab X), and is higher that that of antibody Y (Mab Y). The percent (%) aggregate content of formulation determined by SEC at various time points is displayed in a chart.

FIG. 8. Stability of 13H5 formulations at 5° C. The percent (%) aggregate content of formulation determined by SEC at various time points is displayed in a chart.

FIG. 9. Stability of 13H5, antibody Y (Mab Y), and antibody X (Mab X) formulations at 5° C. The percent (%) aggregate content of formulation determined by SEC at various time intervals is displayed in a chart.

FIG. 10. Stability of 13H5 formulations at 40° C. Antibody degradation over time is ascertained by measuring the total concentration of degradation products (percent (%) fragment) via RF-HPLC. Results obtained with antibody Y (Mab Y) are included in the figure for reference purposes.

FIG. 11. Stability of 13H5 (100 mg/ml) in Formulations E and B at 5° C. Antibody degradation over time is ascertained by measuring the total concentration of degradation products (percent (%) fragment) via RF-HPLC. Results obtained with antibody Y (Mab Y) are included in the figure for reference purposes.

FIG. 12. Stability of 13H5 (100 mg/ml) formulations at pH 6.0, 40° C. Antibody degradation over time is monitored by determining the Mono Q ion exchange chromatography column elution profile of various antibody formulations; percent (%) of protein eluted before the intact antibody peak is displayed in a chart.

FIG. 13. Stability of 13H5 (100 mg/ml) formulations at 5° C. Antibody degradation over time is monitored by determining the Mono Q ion exchange chromatography column elution profile of various antibody formulations; percent (%) of protein eluted before the intact antibody peak is plotted. Results obtained with antibody Z (Mab Z) are included in the figure for reference purposes.

FIG. 14. Stability of 13H5 (100 mg/ml) formulations. The visual appearance of various formulations is determined by the naked eye. Formulations were stored at 5° C. for 9 months.

FIG. 15. Stability of 125 mg/ml, 150 mg/ml, 175 mg/ml and 200 mg/ml 13H5 formulations at 40° C. The percent (%) aggregate content of formulation determined by SEC at various time points is displayed in a chart.

FIG. 16. Stability of 125 mg/ml, 150 mg/ml, 175 mg/ml and 200 mg/ml 13H5 formulations at 5° C. The percent (%) aggregate content of formulation determined by SEC at various time points is displayed in a chart.

FIG. 17. Stability of 125 mg/ml, 150 mg/ml, 175 mg/ml and 200 mg/ml 13H5 formulations at 40° C. Antibody degradation over time is monitored by determining the Dionex ion exchange chromatography column elution profile of various antibody formulations; percent (%) of protein eluted before the intact antibody peak is displayed in a chart.

FIG. 18. Stability of 125 mg/ml, 150 mg/ml, 175 mg/ml and 200 mg/ml 13H5 formulations at 5° C. Antibody degradation over time is monitored by determining the Dionex ion exchange chromatography column elution profile of various antibody formulations; percent (%) of protein eluted before the intact antibody peak is displayed in a chart.

FIG. 19. Listing of pre-filled syringes tested with a formulation of the invention. A glass vial control was included in all stability experiment.

FIG. 20. Stability of 13H5 liquid formulation in pre-filled syringes. Formulation-filled syringes were stored at 5° C. for the indicated length of time. Visual appearance of the formulation was determined by naked eye.

FIG. 21. Stability of 13H5 liquid formulation in pre-filled syringes. Formulation-filled syringes were stored at 5° C. for the indicated length of time. Visual appearance of the formulation was determined by naked eye.

FIG. 22. Stability of 13H5 liquid formulation in pre-filled syringes. Formulation-filled syringes were stored at 40° C. or 25° C. for the indicated length of time. Visual appearance of the formulation was determined by naked eye.

FIG. 23. Stability of 13H5 liquid formulation in Daikyo CZ 1 mL Luer Lock syringe. Formulation-filled syringes were stored at 5° C. for the indicated length of time. The percent (%) monomer content of the formulation determined by SEC at various time points is displayed in a chart.

FIG. 24. Stability of 13H5 liquid formulation in Daikyo CZ 1 mL Luer Lock syringe. Formulation-filled syringes were stored at 25° C. for the indicated length of time. The percent (%) monomer content of the formulation determined by SEC at various time points is displayed in a chart.

FIG. 25. S Stability of 13H5 liquid formulation in Daikyo CZ 1 mL Luer Lock syringe. Formulation-filled syringes were stored at 40° C. for the indicated length of time. The percent (%) monomer content of the formulation determined by SEC at various time points is displayed in a chart.

FIG. 26. Stability of 13H5 liquid formulation in Daikyo CZ 1 mL Luer Lock syringe. Formulation-filled syringes were stored at 5° C. for the indicated length of time. The percent deamidated antibody content (% pre-peak) of the formulation determined by IEC at various time points is displayed in a chart.

### 5. DETAILED DESCRIPTION

The present invention relates to stable, high concentration liquid formulations of antibodies or fragments thereof that specifically bind to a human interferon alpha polypeptide. In certain embodiments, a stable high concentration liquid formulation of an anti-interferon alpha antibody or a fragment thereof is suitable for parenteral administration to a human subject. In a specific embodiment, a stable high concentration liquid formulation of the invention is suitable for subcutaneous administration to a human subject.

### 5.1. Antibody Formulations

In specific embodiments, the present invention encompasses stable liquid formulations of antibodies that specifically bind to an interferon alpha polypeptide, which exhibit low to undetectable levels of antibody aggregation and/or fragmentation with very little to no loss of the biological activities during manufacture, preparation, transportation, and long periods of storage. The present invention also encompasses stable liquid formulations of antibodies that specifically bind to an interferon alpha polypeptide and have increased in vivo half-lives, said formulations exhibiting low to undetectable levels of antibody aggregation and/or fragmentation, and very little to no loss of the biological activities of the antibodies.

In one embodiment, a liquid formulation of the invention is an aqueous formulation. In a specific embodiment, a liquid formulation of the invention is an aqueous formulation wherein the aqueous carrier is distilled water.

In one embodiment, a formulation of the invention is sterile.

In one embodiment, a formulation of the invention is homogeneous.

In one embodiment, a formulation of the invention is isotonic.

The present invention provides stable high concentration liquid formulations of the 13H5, 13H7, and 7H9 anti-human interferon alpha antibodies (see, US Patent Publication 2007/0014724A1).

In one embodiment, a formulation of the invention comprises the 13H5 antibody or a fragment thereof, wherein said antibody or a fragment thereof comprises a VH domain having the amino acid sequence of SEQ ID NO:2 and a VL domain having the amino acid sequence of SEQ ID NO:7. In a specific embodiment, a formulation of the invention comprises the 13H5 antibody, wherein said antibody comprises a heavy chain having the amino acid sequence of SEQ ID NO:1 and a light chain having the amino acid sequence of SEQ ID NO:6. In another embodiment, a formulation of the invention comprises the 13H7 antibody or a fragment thereof, wherein said antibody or a fragment thereof comprises a VH domain having the amino acid sequence of SEQ ID NO:11 and a VL domain having the amino acid sequence of SEQ ID NO: 15. In a further embodiment, a formulation of the invention comprises the 7H9 antibody or a fragment thereof, wherein said antibody or a fragment thereof comprises a VH domain having the amino acid sequence of SEQ ID NO: 19 and a VL domain having the amino acid sequence of SEQ ID NO:23.

The invention encompasses stable liquid formulations comprising a single antibody of interest (including antibody fragment thereof), for example, an antibody that specifically binds to an interferon alpha polypeptide. The invention also encompasses stable liquid formulations comprising two or more antibodies of interest (including antibody fragments thereof), for example, antibodies that specifically bind to an interferon alpha polypeptide(s). In a specific embodiment, a stable liquid formulation of the invention comprises 13H5, 13H7 or 7H9 or a fragment thereof that specifically binds to an interferon alpha polypeptide. In another embodiment, a stable liquid formulation of the invention comprises two or more antibodies (including antibody fragments thereof) that specifically bind to an interferon alpha polypeptide, wherein one of the antibodies (including antibody fragments thereof) is 13H5, 13H7 or 7H9 or an antigen-binding fragment thereof.

In one embodiment, a formulation of the invention comprises at least about 1 mg/ml, at least about 5 mg/ml, at least about 10 mg/ml, at least about 20 mg/ml, at least about 30 mg/ml, at least about 40 mg/ml, at least about 50 mg/ml, at least about 60 mg/ml, at least about 70 mg/ml, at least about 80 mg/ml, at least about 90 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml of an anti-interferon alpha antibody or a fragment thereof. In a specific embodiment, a formulation of the invention comprises at least about 100 mg/ml of an anti-interferon alpha antibody of a fragment thereof. In a specific embodiment, a formulation of the invention comprises at least about 125 mg/ml of an anti-interferon alpha antibody of a fragment thereof. In a specific embodiment, a formulation of the invention comprises at least about 150 mg/ml of an anti-interferon alpha antibody of a fragment thereof. In a specific embodiment, a formulation of the invention comprises at least about 175 mg/ml of an anti-interferon alpha antibody of a fragment thereof. In a specific embodiment, a formulation of the invention comprises at least about 200 mg/ml of an anti-interferon alpha antibody of a fragment thereof. In another embodiment, a formulation of the invention comprises between about 1 mg/ml and about 25 mg/ml, between about 1 mg/ml and about 200 mg/ml, between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml of an anti-interferon alpha antibody or a fragment thereof. In a specific embodiment, a formulation of the invention comprises between about 90 mg/ml and about 110 mg/ml of an anti-interferon alpha antibody or a fragment thereof. In a specific embodiment, a formulation of the invention comprises between about 100 mg/ml and about 210 mg/ml of an anti-interferon alpha antibody or a fragment thereof. In a further embodiment, a formulation described herein comprises about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 250 mg/ml, or about 300 mg/ml of an anti-interpheron alpha antibody or a fragment thereof. In a specific embodiment, a formulation of the invention comprises about 100 mg/ml of an anti-interferon alpha antibody or a fragment thereof. In a specific embodiment, a formulation of the invention comprises about 125 mg/ml of an anti-interferon alpha antibody or a fragment thereof. In a specific embodiment, a formulation of the invention comprises about 150 mg/ml of an anti-interferon alpha antibody or a fragment thereof. In a specific embodiment, a formulation of the invention comprises about 175 mg/ml of an anti-interferon alpha antibody or a fragment thereof. In a specific embodiment, a formulation of the invention comprises about 200 mg/ml of an anti-interferon alpha antibody or a fragment thereof.

In one embodiment, a formulation of the invention comprises at least 1 mg/ml, at least 5 mg/ml, at least 10 mg/ml, at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml, at least 200 mg/ml, at least 250 mg/ml, or at least 300 mg/ml of an anti-interferon alpha antibody or a fragment thereof. In a specific embodiment, a formulation of the invention comprises at least 100 mg/ml of an anti-interferon alpha antibody of a fragment thereof. In a specific embodiment, a formulation of the invention comprises at least 125 mg/ml of an anti-interferon alpha antibody of a fragment thereof. In a specific embodiment, a formulation of the invention comprises at least 150 mg/ml of an anti-interferon alpha antibody of a fragment thereof. In a specific embodiment, a formulation of the invention comprises at least 175 mg/ml of an anti-interferon alpha antibody of a fragment thereof. In a specific embodiment, a formulation of the invention comprises at least 200 mg/ml of an anti-interferon alpha antibody of a fragment thereof. In another embodiment, a formulation of the invention comprises between 1 mg/ml and 25 mg/ml, between 1 mg/ml and 200 mg/ml, between 25 mg/ml and 200 mg/ml, between 50 mg/ml and 200 mg/ml, between 75 mg/ml and 200 mg/ml, between 100 mg/ml and 200 mg/ml, between 125 mg/ml and 200 mg/ml, between 150 mg/ml and 200 mg/ml, between 25 mg/ml and 150 mg/ml, between 50 mg/ml and 150 mg/ml, between 75 mg/ml and 150 mg/ml, between 100 mg/ml and 150 mg/ml, between 125 mg/ml and 150 mg/ml, between 25 mg/ml and 125 mg/ml, between 50 mg/ml and 125 mg/ml, between 75 mg/ml and 125 mg/ml, between 100 mg/ml and 125 mg/ml, between 25 mg/ml and 100 mg/ml, between 50 mg/ml and 100 mg/ml, between 75 mg/ml and 100 mg/ml, between 25 mg/ml and 75 mg/ml, between 50 mg/ml and 75 mg/ml, or between 25 mg/ml and 50 mg/ml of an anti-interferon alpha antibody or a fragment thereof. In a specific embodiment, a formulation of the invention comprises between 90 mg/ml and 110 mg/ml of an anti-interferon alpha antibody or a fragment thereof. In a specific embodiment, a formulation of the invention comprises between 100 mg/ml and 210 mg/ml of an anti-interferon alpha antibody or a fragment thereof. In a further embodiment, a formulation described herein comprises 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 250 mg/ml, or 300 mg/ml of an anti-interpheron alpha antibody or a fragment thereof. In a specific embodiment, a formulation of the invention comprises 100 mg/ml of an anti-interferon alpha antibody or a fragment thereof. In a specific embodiment, a formulation of the invention comprises 125 mg/ml of an anti-interferon alpha antibody or a fragment thereof. In a specific embodiment, a formulation of the invention comprises 150 mg/ml of an anti-interferon alpha antibody or a fragment thereof. In a specific embodiment, a formulation of the invention comprises 175 mg/ml of an anti-interferon alpha antibody or a fragment thereof. In a specific embodiment, a formulation of the invention comprises 200 mg/ml of an anti-interferon alpha antibody or a fragment thereof.

In one embodiment, a formulation of the invention comprises at least about 20 mg/ml, at least about 30 mg/ml, at least about 40 mg/ml, at least about 50 mg/ml, at least about 60 mg/ml, at least about 70 mg/ml, at least about 80 mg/ml, at least about 90 mg/ml, at least about 100 mg/ml, at least about 110 mg/ml, at least about 120 mg/ml, at least about 130 mg/ml, at least about 140 mg/ml, at least about 150 mg/ml, at least about 160 mg/ml, at least about 170 mg/ml, at least about 180 mg/ml, at least about 190 mg/ml, at least about 200 mg/ml, at least about 250 mg/ml, or at least about 300 mg/ml of a 13H5 anti-interferon alpha antibody. In a specific embodiment, a formulation of the invention comprises at least about 100 mg/ml of a 13H5 anti-interferon alpha antibody. In a specific embodiment, a formulation of the invention comprises at least about 125 mg/ml of a 13H5 anti-interferon alpha antibody. In a specific embodiment, a formulation of the invention comprises at least about 150 mg/ml of a 13H5 anti-interferon alpha antibody. In a specific embodiment, a formulation of the invention comprises at least about 175 mg/ml of a 13H5 anti-interferon alpha antibody. In a specific embodiment, a formulation of the invention comprises at least about 200 mg/ml of a 13H5 anti-interferon alpha antibody. In another embodiment, a formulation of the invention comprises between about 25 mg/ml and about 200 mg/ml, between about 50 mg/ml and about 200 mg/ml, between about 75 mg/ml and about 200 mg/ml, between about 100 mg/ml and about 200 mg/ml, between about 125 mg/ml and about 200 mg/ml, between about 150 mg/ml and about 200 mg/ml, between about 25 mg/ml and about 150 mg/ml, between about 50 mg/ml and about 150 mg/ml, between about 75 mg/ml and about 150 mg/ml, between about 100 mg/ml and about 150 mg/ml, between about 125 mg/ml and about 150 mg/ml, between about 25 mg/ml and about 125 mg/ml, between about 50 mg/ml and about 125 mg/ml, between about 75 mg/ml and about 125 mg/ml, between about 100 mg/ml and about 125 mg/ml, between about 25 mg/ml and about 100 mg/ml, between about 50 mg/ml and about 100 mg/ml, between about 75 mg/ml and about 100 mg/ml, between about 25 mg/ml and about 75 mg/ml, between about 50 mg/ml and about 75 mg/ml, or between about 25 mg/ml and about 50 mg/ml of a 13H5 anti-interferon alpha antibody. In a specific embodiment, a formulation of the invention comprises between about 90 mg/ml and about 110 mg/ml of a 13H5 anti-interferon alpha antibody. In a specific embodiment, a formulation of the invention comprises between about 100 mg/ml and about 210 mg/ml of a 13H5 anti-interferon alpha antibody. In a further embodiment, a formulation described herein comprises about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 110 mg/ml, about 120 mg/ml, about 130 mg/ml, about 140 mg/ml, about 150 mg/ml, about 160 mg/ml, about 170 mg/ml, about 180 mg/ml, about 190 mg/ml, about 200 mg/ml, about 250 mg/ml, or about 300 mg/ml of a 13H5 anti-interpheron alpha antibody. In a specific embodiment, a formulation of the invention comprises about 100 mg/ml of a 13H5 anti-interferon alpha antibody.

In one embodiment, a formulation of the invention comprises at least 20 mg/ml, at least 30 mg/ml, at least 40 mg/ml, at least 50 mg/ml, at least 60 mg/ml, at least 70 mg/ml, at least 80 mg/ml, at least 90 mg/ml, at least 100 mg/ml, at least 110 mg/ml, at least 120 mg/ml, at least 130 mg/ml, at least 140 mg/ml, at least 150 mg/ml, at least 160 mg/ml, at least 170 mg/ml, at least 180 mg/ml, at least 190 mg/ml, at least 200 mg/ml, at least 250 mg/ml, or at least 300 mg/ml of a 13H5 anti-interferon alpha antibody. In a specific embodiment, a formulation of the invention comprises at least 100 mg/ml of a 13H5 anti-interferon alpha antibody. In a specific embodiment, a formulation of the invention comprises at least 125 mg/ml of a 13H5 anti-interferon alpha antibody. In a specific embodiment, a formulation of the invention comprises at least 150 mg/ml of a 13H5 anti-interferon alpha antibody. In a specific embodiment, a formulation of the invention comprises at least 175 mg/ml of a 13H5 anti-interferon alpha antibody. In a specific embodiment, a formulation of the invention comprises at least 200 mg/ml of a 13H5 anti-interferon alpha antibody. In another embodiment, a formulation of the invention comprises between 25 mg/ml and 200 mg/ml, between 50 mg/ml and 200 mg/ml, between 75 mg/ml and 200 mg/ml, between 100 mg/ml and 200 mg/ml, between 125 mg/ml and 200 mg/ml, between 150 mg/ml and 200 mg/ml, between 25 mg/ml and 150 mg/ml, between 50 mg/ml and 150 mg/ml, between 75 mg/ml and 150 mg/ml, between 100 mg/ml and 150 mg/ml, between 125 mg/ml and 150 mg/ml, between 25 mg/ml and 125 mg/ml, between 50 mg/ml and 125 mg/ml, between 75 mg/ml and 125 mg/ml, between 100 mg/ml and 125 mg/ml, between 25 mg/ml and 100 mg/ml, between 50 mg/ml and 100 mg/ml, between 75 mg/ml and 100 mg/ml, between 25 mg/ml and 75 mg/ml, between 50 mg/ml and 75 mg/ml, or between 25 mg/ml and 50 mg/ml of a 13H5 anti-interferon alpha antibody. In a specific embodiment, a formulation of the invention comprises between 90 mg/ml and 110 mg/ml of a 13H5 anti-interferon alpha antibody. In a specific embodiment, a formulation of the invention comprises between 100 mg/ml and 210 mg/ml of a 13H5 anti-interferon alpha antibody. In a further embodiment, a formulation described herein comprises 20 mg/ml, 30 mg/ml, 40 mg/ml, 50 mg/ml, 60 mg/ml, 70 mg/ml, 80 mg/ml, 90 mg/ml, 100 mg/ml, 110 mg/ml, 120 mg/ml, 130 mg/ml, 140 mg/ml, 150 mg/ml, 160 mg/ml, 170 mg/ml, 180 mg/ml, 190 mg/ml, 200 mg/ml, 250 mg/ml, or 300 mg/ml of a 13H5 anti-interpheron alpha antibody. In a specific embodiment, a formulation of the invention comprises 100 mg/ml of a 13H5 anti-interferon alpha antibody.

Optionally, the formulations of the invention may further comprise common excipients and/or additives such as buffering agents, saccharides, salts and surfactants. Additionally or alternatively, the formulations of the invention may further comprise common excipients and/or additives, such as, but not limited to, solubilizers, diluents, binders, stabilizers, salts, lipophilic solvents, amino acids, chelators, preservatives, or the like.

In certain embodiments, the buffering agent is selected from the group consisting of histidine, citrate, phosphate, glycine, and acetate. In other embodiments the saccharide excipient is selected from the group consisting of trehalose, sucrose, mannitol, maltose and raffinose. In still other embodiments the surfactant is selected from the group consisting of polysorbate 20, polysorbate 40, polysorbate 80, and Pluronic F68. In yet other embodiments the salt is selected from the group consisting of NaCl, KCl, MgCl₂, and CaCl₂

Optionally, the formulations of the invention may further comprise other common auxiliary components, such as, but not limited to, suitable excipients, polyols, solubilizers, diluents, binders, stabilizers, lipophilic solvents, chelators, preservatives, or the like.

The formulations of the invention include a buffering or pH adjusting agent to provide improved pH control. In one embodiment, a formulation of the invention has a pH of between about 3.0 and about 9.0, between about 4.0 and about 8.0, between about 5.0 and about 8.0, between about 5.0 and about 7.0, between about 5.0 and about 6.5, between about 5.5 and about 8.0, between about 5.5 and about 7.0, or between about 5.5 and about 6.5. In a further embodiment, a formulation of the invention has a pH of about 3.0, about 3.5, about 4.0, about 4.5, about 5.0, about 5.1, about 5.2, about 5.3, about 5.4, about 5.5, about 5.6, about 5.7, about 5.8, about 5.9, about 6.0, about 6.1, about 6.2, about 6.3, about 6.4, about 6.5, about 6.6, about 6.7, about 6.8, about 6.9, about 7.0, about 7.5, about 8.0, about 8.5, or about 9.0. In a specific embodiment, a formulation of the invention has a pH of about 6.0.

The formulations of the invention include a buffering or pH adjusting agent to provide improved pH control. In one embodiment, a formulation of the invention has a pH of between 3.0 and 9.0, between 4.0 and 8.0, between 5.0 and 8.0, between 5.0 and 7.0, between 5.0 and 6.5, between 5.5 and 8.0, between 5.5 and 7.0, or between 5.5 and 6.5. In a further embodiment, a formulation of the invention has a pH of 3.0, 3.5, 4.0, 4.5, 5.0, 5.1, 5.2, 5.3, 5.4, 5.5, 5.6, 5.7, 5.8, 5.9, 6.0, 6.1, 6.2, 6.3, 6.4, 6.5, 6.6, 6.7, 6.8, 6.9, 7.0, 7.5, 8.0, 8.5, or 9.0. In a specific embodiment, a formulation of the invention has a pH of 6.0.

The pH of the formulation generally should not be equal to the isoelectric point of the particular antibody (including antibody fragment thereof) to be used in the formulation (for example, but not limited to, the isoelectric point of 13H5, 13H7 or 7H9) and may range from about 4.0 to about 8.0, or may range from about 5.5 to about 6.5.

The pH of the formulation generally should not be equal to the isoelectric point of the particular antibody (including antibody fragment thereof) to be used in the formulation (for example, but not limited to, the isoelectric point of 13H5, 13H7 or 7H9) and may range from 4.0 to 8.0, or may range from 5.5 to 6.5.

Typically, the buffering agent is a salt prepared from an organic or inorganic acid or base. Representative buffering agents include, but are not limited to, organic acid salts such as salts of citric acid, ascorbic acid, gluconic acid, carbonic acid, tartaric acid, succinic acid, acetic acid, or phthalic acid; Tris, tromethamine hydrochloride, or phosphate buffers. In addition, amino acid components can also function in a buffering capacity. Representative amino acid components which may be utilized in the formulations of the invention as buffering agents include, but are not limited to, glycine and histidine. In certain embodiments, the buffering agent is selected from the group consisting of histidine, citrate, phosphate, glycine, and acetate. In a specific embodiment, the buffering agent is histidine. In another specific embodiment, the buffering agent is citrate. The purity of the buffering agent should be at least 98%, or at least 99%, or at least 99.5%. As used herein, the term “purity” in the context of histidine refers to chemical purity of histidine as understood in the art, e.g., as described in The Merck Index, 13^{th} ed., O'Neil et al. ed. (Merck & Co., 2001).

Buffering agents are typically used at concentrations between about 1 mM and about 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of about 1 mM, or of about 5 mM, or of about 10 mM, or of about 15 mM, or of about 20 mM, or of about 25 mM, or of about 30 mM, or of about 35 mM, or of about 40 mM, or of about 45 mM, or of about 50 mM, or of about 60 mM, or of about 70 mM, or of about 80 mM, or of about 90 mM, or of about 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between about 10 mM and about 50 mM. In another specific embodiment, the buffering agent is at a concentration of between 10 mM and 50 mM.

Buffering agents are typically used at concentrations between 1 mM and 200 mM or any range or value therein, depending on the desired ionic strength and the buffering capacity required. The usual concentrations of conventional buffering agents employed in parenteral formulations can be found in: Pharmaceutical Dosage Form: Parenteral Medications, Volume 1, 2^{nd} Edition, Chapter 5, p. 194, De Luca and Boylan, “Formulation of Small Volume Parenterals”, Table 5: Commonly used additives in Parenteral Products. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In one embodiment, the buffering agent is at a concentration of 1 mM, or of 5 mM, or of 10 mM, or of 15 mM, or of 20 mM, or of 25 mM, or of 30 mM, or of 35 mM, or of 40 mM, or of 45 mM, or of 50 mM, or of 60 mM, or of 70 mM, or of 80 mM, or of 90 mM, or of 100 mM. In a specific embodiment, the buffering agent is at a concentration of between 10 mM and 50 mM. In another specific embodiment, the buffering agent is at a concentration of between 10 mM and 50 mM.

In certain embodiments, a formulation of the invention comprises a buffering agent. In one embodiment, said buffering agent is selected from the group consisting of histidine, citrate, phosphate, glycine, and acetate. In a specific embodiment, a formulation of the invention comprises histidine as a buffering agent. In a further embodiment, a formulation of the invention comprises a citrate buffer.

In one embodiment, a formulation of the invention comprises at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM histidine. In another embodiment, a formulation of the invention comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM histidine. In a further embodiment of the invention comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM histidine. In a specific embodiment, a formulation of the invention comprises about 25 mM histidine.

In one embodiment, a formulation of the invention comprises at least 1 mM, at least 5 mM, at least 10 mM, at least 20 mM, at least 30 mM, at least 40 mM, at least 50 mM, at least 75 mM, at least 100 mM, at least 150 mM, or at least 200 mM histidine. In another embodiment, a formulation of the invention comprises between 1 mM and 200 mM, between 1 mM and 150 mM, between 1 mM and 100 mM, between 1 mM and 75 mM, between 10 mM and 200 mM, between 10 mM and 150 mM, between 10 mM and 100 mM, between 10 mM and 75 mM, between 10 mM and 50 mM, between 10 mM and 40 mM, between 10 mM and 30 mM, between 20 mM and 75 mM, between 20 mM and 50 mM, between 20 mM and 40 mM, or between 20 mM and 30 mM histidine. In a further embodiment of the invention comprises 1 mM, 5 mM, 10 mM, 20 mM, 25 mM, 30 mM, 35 mM, 40 mM, 45 mM, 50 mM, 60 mM, 70 mM, 80 mM, 90 mM, 100 mM, 150 mM, or 200 mM histidine. In a specific embodiment, a formulation of the invention comprises 25 mM histidine.

In one embodiment, a formulation of the invention comprises at least about 1 mM, at least about 5 mM, at least about 10 mM, at least about 20 mM, at least about 30 mM, at least about 40 mM, at least about 50 mM, at least about 75 mM, at least about 100 mM, at least about 150 mM, or at least about 200 mM citrate buffer. In another embodiment, a formulation of the invention comprises between about 1 mM and about 200 mM, between about 1 mM and about 150 mM, between about 1 mM and about 100 mM, between about 1 mM and about 75 mM, between about 10 mM and about 200 mM, between about 10 mM and about 150 mM, between about 10 mM and about 100 mM, between about 10 mM and about 75 mM, between about 10 mM and about 50 mM, between about 10 mM and about 40 mM, between about 10 mM and about 30 mM, between about 20 mM and about 75 mM, between about 20 mM and about 50 mM, between about 20 mM and about 40 mM, or between about 20 mM and about 30 mM citrate buffer. In a further embodiment of the invention comprises about 1 mM, about 5 mM, about 10 mM, about 20 mM, about 25 mM, about 30 mM, about 35 mM, about 40 mM, about 45 mM, about 50 mM, about 60 mM, about 70 mM, about 80 mM, about 90 mM, about 100 mM, about 150 mM, or about 200 mM citrate buffer. In a specific embodiment, a formulation of the invention comprises about 20 mM citrate buffer.

In one embodiment, a formulation of the invention comprises at least 1 mM, at least 5 mM, at least 10 mM, at least 20 mM, at least 30 mM, at least 40 mM, at least 50 mM, at least 75 mM, at least 100 mM, at least 150 mM, or at least 200 mM citrate buffer. In another embodiment, a formulation of the invention comprises between 1 mM and 200 mM, between 1 mM and 150 mM, between 1 mM and 100 mM, between 1 mM and 75 mM, between 10 mM and 200 mM, between 10 mM and 150 mM, between 10 mM and 100 mM, between 10 mM and 75 mM, between 10 mM and 50 mM, between 10 mM and 40 mM, between 10 mM and 30 mM, between 20 mM and 75 mM, between 20 mM and 50 mM, between 20 mM and 40 mM, or between 20 mM and 30 mM citrate buffer. In a further embodiment of the invention comprises 1 mM, 5 mM, 10 mM, 20 mM, 25 mM, 30 mM, 35 mM, 40 mM, 45 mM, 50 mM, 60 mM, 70 mM, 80 mM, 90 mM, 100 mM, 150 mM, or 200 mM citrate buffer. In a specific embodiment, a formulation of the invention comprises 20 mM citrate buffer.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between about 1% to about 99% by weight or volume. In one embodiment, the carbohydrate excipient is present at between about 0.1% to about 20%. In another embodiment, the carbohydrate excipient is present at between about 0.1% to about 15%. In a specific embodiment, the carbohydrate excipient is present at between about 0.1% to about 5%, or between about 1% to about 20%, or between about 5% to about 15%, or between about 8% to about 10%, or between about 10% and about 15%, or between about 15% and about 20%. In another specific embodiment, the carbohydrate excipient is present at between 0.1% to 20%, or between 5% to 15%, or between 8% to 10%, or between 10% and 15%, or between 15% and 20%. In still another specific embodiment, the carbohydrate excipient is present at between about 0.1% to about 5%. In still another specific embodiment, the carbohydrate excipient is present at between about 5% to about 10%. In yet another specific embodiment, the carbohydrate excipient is present at between about 15% to about 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

In certain embodiments, the formulations of the invention comprise a carbohydrate excipient. Carbohydrate excipients can act, e.g., as viscosity enhancing agents, stabilizers, bulking agents, solubilizing agents, and/or the like. Carbohydrate excipients are generally present at between 1% to 99% by weight or volume. In one embodiment, the carbohydrate excipient is present at between 0.1% to 20%. In another embodiment, the carbohydrate excipient is present at between 0.1% to 15%. In a specific embodiment, the carbohydrate excipient is present at between 0.1% to 5%, or between 1% to 20%, or between 5% to 15%, or between 8% to 10%, or between 10% and 15%, or between 15% and 20%. In another specific embodiment, the carbohydrate excipient is present at between 0.1% to 20%, or between 5% to 15%, or between 8% to 10%, or between 10% and 15%, or between 15% and 20%. In still another specific embodiment, the carbohydrate excipient is present at between 0.1% to 5%. In still another specific embodiment, the carbohydrate excipient is present at between 5% to 10%. In yet another specific embodiment, the carbohydrate excipient is present at between 15% to 20%. In still other specific embodiments, the carbohydrate excipient is present at 1%, or at 1.5%, or at 2%, or at 2.5%, or at 3%, or at 4%, or at 5%, or at 10%, or at 15%, or at 20%.

Carbohydrate excipients suitable for use in the formulations of the invention include, for example, monosaccharides such as fructose, maltose, galactose, glucose, D-mannose, sorbose, and the like; disaccharides, such as lactose, sucrose, trehalose, cellobiose, and the like; polysaccharides, such as raffinose, melezitose, maltodextrins, dextrans, starches, and the like; and alditols, such as mannitol, xylitol, maltitol, lactitol, xylitol sorbitol (glucitol) and the like. In one embodiment, the carbohydrate excipients for use in the present invention are selected from the group consisting of, sucrose, trehalose, lactose, mannitol, and raffinose. In a specific embodiment, the carbohydrate excipient is trehalose. In another specific embodiment, the carbohydrate excipient is mannitol. In yet another specific embodiment, the carbohydrate excipient is sucrose. In still another specific embodiment, the carbohydrate excipient is raffinose. The purity of the carbohydrate excipient should be at least 98%, or at least 99%, or at least 99.5%.

In one embodiment, a formulation of the invention comprises at least about 1%, at least about 2%, at least about 4%, at least about 8%, at least about 20%, at least about 30%, or at least about 40% trehalose. In another embodiment, a formulation of the invention comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the invention comprises about 1%, about 2%, about 4%, about 8%, about 20%, about 30%, or about 40% trehalose. In a specific embodiment, a formulation of the invention comprises about 8% trehalose.

In one embodiment, a formulation of the invention comprises at least about 1%, at least about 2%, at least about 4%, at least about 5%, at least about 6%, at least about 7%, at least about 8%, at least about 10%, at least about 20%, at least about 30%, or at least about 40% sucrose. In another embodiment, a formulation of the invention comprises between about 1% and about 40%, between about 1% and about 30%, between about 1% and about 20%, between about 2% and about 40%, between about 2% and about 30%, between about 2% and about 20%, between about 4% and about 40%, between about 4% and about 30%, or between about 4% and about 20% trehalose. In a further embodiment, a formulation of the invention comprises about 1%, about 2%, about 4%, about 5%, about 6%, about 7%, about 8%, about 9%, about 10%, about 20%, about 30%, or about 40% sucrose. In a specific embodiment, a formulation of the invention comprises about 5% sucrose.

In one embodiment, a formulation of the invention comprises a polyol. In a further embodiment, a formulation of the invention comprises mannitol. In one embodiment, a formulation of the invention comprises at least about 0.1%, at least about 0.25%, at least about 0.5%, at least about 1%, at least about 1.5%, at least about 3%, at least about 6%, at least about 10%, or at least about 20% mannitol. In another embodiment, a formulation of the invention comprises between about 0.1% and about 20%, between about 0.1% and about 10%, between about 0.1% and about 6%, between about 0.1% and about 3%, between about 0.25% and about 20%, between about 0.25% and about 10%, between about 0.25% and about 6%, between about 0.25% and about 3%, between about 0.5% and about 20%, between about 0.5% and about 10%, between about 0.5% and about 6%, between about 0.5% and about 3%, between about 1% and about 20%, between about 1% and about 10%, between about 1% and about 6%, or between about 1% and about 3% mannitol. In a further embodiment, a formulation of the invention comprises about 0.1%, about 0.25%, about 0.5%, about 1%, about 1.5%, about 3%, about 6%, about 10%, or about 20% mannitol. In a specific embodiment, a formulation of the invention comprises about 1.5% mannitol.

In one embodiment, a formulation of the invention comprises at least 1%, at least 2%, at least 4%, at least 8%, at least 20%, at least 30%, or at least 40% trehalose. In another embodiment, a formulation of the invention comprises between 1% and 40%, between 1% and 30%, between 1% and 20%, between 2% and 40%, between 2% and 30%, between 2% and 20%, between 4% and 40%, between 4% and 30%, or between 4% and 20% trehalose. In a further embodiment, a formulation of the invention comprises 1%, 2%, 4%, 8%, 20%, 30%, or 40% trehalose. In a specific embodiment, a formulation of the invention comprises 8% trehalose.

In one embodiment, a formulation of the invention comprises at least 1%, at least 2%, at least 4%, at least 5%, at least 6%, at least 7%, at least 8%, at least 10%, at least 20%, at least 30%, or at least 40% sucrose. In another embodiment, a formulation of the invention comprises between 1% and 40%, between 1% and 30%, between 1% and 20%, between 2% and 40%, between 2% and 30%, between 2% and 20%, between 4% and 40%, between 4% and 30%, or between 4% and 20% trehalose. In a further embodiment, a formulation of the invention comprises 1%, 2%, 4%, 5%, 6%, 7%, 8%, 9%, 10%, 20%, 30%, or 40% sucrose. In a specific embodiment, a formulation of the invention comprises 5% sucrose.

In one embodiment, a formulation of the invention comprises a polyol. In a further embodiment, a formulation of the invention comprises mannitol. In one embodiment, a formulation of the invention comprises at least 0.1%, at least 0.25%, at least 0.5%, at least 1%, at least 1.5%, at least 3%, at least 6%, at least 10%, or at least 20% mannitol. In another embodiment, a formulation of the invention comprises between 0.1% and 20%, between 0.1% and 10%, between 0.1% and 6%, between 0.1% and 3%, between 0.25% and 20%, between 0.25% and 10%, between 0.25% and 6%, between 0.25% and 3%, between 0.5% and 20%, between 0.5% and 10%, between 0.5% and 6%, between 0.5% and 3%, between 1% and 20%, between 1% and 10%, between 1% and 6%, or between 1% and 3% mannitol. In a further embodiment, a formulation of the invention comprises 0.1%, 0.25%, 0.5%, about 1%, 1.5%, 3%, 6%, 10%, or 20% mannitol. In a specific embodiment, a formulation of the invention comprises 1.5% mannitol.

In one embodiment, a formulation of the invention comprises an excipient. In a specific embodiment, a formulation of the invention comprises at least one excipient selected from the group consisting of: sugar, salt, surfactant, amino acid, polyol, chelating agent, emulsifier and preservative. In one embodiment, a formulation of the invention comprises a salt. In one embodiment, a formulation of the invention comprises a salt selected from the group consisting of: NaCl, KCl, CaCl₂, and MgCl₂. In a specific embodiment, a formulation of the invention comprises NaCl.

In one embodiment, a formulation of the invention comprises at least about 10 mM, at least about 25 mM, at least about 50 mM, at least about 75 mM, at least about 100 mM, at least about 125 mM, at least about 150 mM, at least about 175 mM. at least about 200 mM, or at least about 300 mM sodium chloride. In a further embodiment, a formulation described herein comprises between about 10 mM and about 300 mM, between about 10 mM and about 200 mM, between about 10 mM and about 175 mM, between about 10 mM and about 150 mM, between about 25 mM and about 300 mM, between about 25 mM and about 200 mM, between about 25 mM and about 175 mM, between about 25 mM and about 150 mM, between about 50 mM and about 300 mM, between about 50 mM and about 200 mM, between about 50 mM and about 175 mM, between about 50 mM and about 150 mM, between about 75 mM and about 300 mM, between about 75 mM and about 200 mM, between about 75 mM and about 175 mM, between about 75 mM and about 150 mM, between about 100 mM and about 300 mM, between about 100 mM and about 200 mM, between about 100 mM and about 175 mM, or between about 100 mM and about 150 mM sodium chloride. In a further embodiment, a formulation of the invention comprises about 10 mM. about 25 mM, about 50 mM, about 75 mM, about 100 mM, about 125 mM, about 150 mM, about 175 mM, about 200 mM, or about 300 mM sodium chloride. In a specific embodiment, a formulation of the invention comprises 125 mM sodium chloride.

In one embodiment, a formulation of the invention comprises at least 10 mM, at least 25 mM, at least 50 mM, at least 75 mM, at least 100 mM, at least 125 mM, at least 150 mM, at least 175 mM. at least 200 mM, or at least 300 mM sodium chloride. In a further embodiment, a formulation described herein comprises between 10 mM and 300 mM, between 10 mM and 200 mM, between 10 mM and 175 mM, between 10 mM and 150 mM, between 25 mM and 300 mM, between 25 mM and 200 mM, between 25 mM and 175 mM, between 25 mM and 150 mM, between 50 mM and 300 mM, between 50 mM and 200 mM, between 50 mM and 175 mM, between 50 mM and 150 mM, between 75 mM and 300 mM, between 75 mM and 200 mM, between 75 mM and 175 mM, between 75 mM and 150 mM, between 100 mM and 300 mM, between 100 mM and 200 mM, between 100 mM and 175 mM, or between 100 mM and 150 mM sodium chloride. In a further embodiment, a formulation of the invention comprises 10 mM. 25 mM, 50 mM, 75 mM, 100 mM, 125 mM, 150 mM, 175 mM, 200 mM, or 300 mM sodium chloride. In a specific embodiment, a formulation of the invention comprises 125 mM sodium chloride.

The formulations of the invention may further comprise a surfactant. The term “surfactant” as used herein refers to organic substances having amphipathic structures; namely, they are composed of groups of opposing solubility tendencies, typically an oil-soluble hydrocarbon chain and a water-soluble ionic group. Surfactants can be classified, depending on the charge of the surface-active moiety, into anionic, cationic, and nonionic surfactants. Surfactants are often used as wetting, emulsifying, solubilizing, and dispersing agents for various pharmaceutical compositions and preparations of biological materials. Pharmaceutically acceptable surfactants like polysorbates (e.g. polysorbates 20 or 80); polyoxamers (e.g. poloxamer 188); Triton; sodium octyl glycoside; lauryl-, myristyl-, linoleyl-, or stearyl-sulfobetaine; lauryl-, myristyl-, linoleyl- or stearyl-sarcosine; linoleyl-, myristyl-, or cetyl-betaine; lauroamidopropyl-, cocamidopropyl-, linoleamidopropyl-, myristamidopropyl-, palmidopropyl-, or isostearamidopropyl-betaine (e.g. lauroamidopropyl); myristamidopropyl-, palmidopropyl-, or isostearamidopropyl-dimethylamine; sodium methyl cocoyl-, or disodium methyl oleyl-taurate; and the MONAQUA™ series (Mona Industries, Inc., Paterson, N.J.), polyethyl glycol, polypropyl glycol, and copolymers of ethylene and propylene glycol (e.g. Pluronics, PF68 etc), can optionally be added to the formulations of the invention to reduce aggregation. Surfactants are particularly useful if a pump or plastic container is used to administer the formulation. The presence of a pharmaceutically acceptable surfactant mitigates the propensity for the protein to aggregate. In a specific embodiment, the formulations of the invention comprise a polysorbate which is at a concentration ranging from between about 0.001% to about 1%, or about 0.001% to about 0.1%, or about 0.01% to about 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80. In a specific embodiment, the formulations of the invention comprise a polysorbate which is at a concentration ranging from between 0.001% to 1%, or 0.001% to 0.1%, or 0.01% to 0.1%. In other specific embodiments, the formulations of the invention comprise a polysorbate which is at a concentration of 0.001%, or 0.002%, or 0.003%, or 0.004%, or 0.005%, or 0.006%, or 0.007%, or 0.008%, or 0.009%, or 0.01%, or 0.015%, or 0.02%. In another specific embodiment, the polysorbate is polysorbate-80.

In one embodiment, a formulation of the invention comprises a surfactant. In one embodiment, a formulation of the invention comprises Polysorbate 20, Polysorbate 40, Polysorbate 60, or Polysorbate 80. In a specific embodiment, a formulation of the invention comprises Polysorbate 80.

In one embodiment, a formulation of the invention comprises at least about 0.001%, at least about 0.002%, at least about 0.005%, at least about 0.01%, at least about 0.02%, at least about 0.05%, at least about 0.1%, at least about 0.2%, or at least about 0.5% Polysorbate 80. In another embodiment, a formulation of the invention comprises between about 0.001% and about 0.5%, between about 0.001% and about 0.2%, between about 0.001% and about 0.1%, between about 0.001% and about 0.05%, between about 0.002% and about 0.5%, between about 0.002% and about 0.2%, between about 0.002% and about 0.1%, between about 0.002% and about 0.05%, between about 0.005% and about 0.5%, between about 0.005% and about 0.2%, between about 0.005% and about 0.1%, between about 0.005% and about 0.05%, between about 0.01% and about 0.5%, between about 0.01% and about 0.2%, between about 0.01% and about 0.1%, or between about 0.01% and about 0.05% Polysorbate 80. In a further embodiment, a formulation of the invention comprises about 0.001%, about 0.002%, about 0.005%, about 0.01%, about 0.02%, about 0.05%, about 0.1%, about 0.2%, and about 0.5% Polysorbate 80. In a specific embodiment, a formulation of the invention comprises about 0.02% Polysorbate 80.

In one embodiment, a formulation of the invention comprises at least 0.001%, at least 0.002%, at least 0.005%, at least 0.01%, at least 0.02%, at least 0.05%, at least 0.1%, at least 0.2%, or at least 0.5% Polysorbate 80. In another embodiment, a formulation of the invention comprises between 0.001% and 0.5%, between 0.001% and 0.2%, between 0.001% and 0.1%, between 0.001% and 0.05%, between 0.002% and 0.5%, between 0.002% and 0.2%, between 0.002% and 0.1%, between 0.002% and 0.05%, between 0.005% and 0.5%, between 0.005% and 0.2%, between 0.005% and 0.1%, between 0.005% and 0.05%, between 0.01% and 0.5%, between 0.01% and 0.2%, between 0.01% and 0.1%, or between 0.01% and 0.05% Polysorbate 80. In a further embodiment, a formulation of the invention comprises 0.001%, 0.002%, 0.005%, 0.01%, 0.02%, 0.05%, 0.1%, 0.2%, and 0.5% Polysorbate 80. In a specific embodiment, a formulation of the invention comprises 0.02% Polysorbate 80.

Optionally, the formulations of the invention may further comprise other common excipients and/or additives including, but not limited to, diluents, binders, stabilizers, lipophilic solvents, preservatives, adjuvants, or the like. Pharmaceutically acceptable excipients and/or additives may be used in the formulations of the invention. Commonly used excipients/additives, such as pharmaceutically acceptable chelators (for example, but not limited to, EDTA, DTPA or EGTA) can optionally be added to the formulations of the invention to reduce aggregation. These additives are particularly useful if a pump or plastic container is used to administer the formulation.

Preservatives, such as phenol, m-cresol, p-cresol, o-cresol, chlorocresol, benzyl alcohol, phenylmercuric nitrite, phenoxyethanol, formaldehyde, chlorobutanol, magnesium chloride (for example, but not limited to, hexahydrate), alkylparaben (methyl, ethyl, propyl, butyl and the like), benzalkonium chloride, benzethonium chloride, sodium dehydroacetate and thimerosal, or mixtures thereof can optionally be added to the formulations of the invention at any suitable concentration such as between about 0.001% to about 5%, or any range or value therein. The concentration of preservative used in the formulations of the invention is a concentration sufficient to yield an microbial effect. Such concentrations are dependent on the preservative selected and are readily determined by the skilled artisan.

Other contemplated excipients/additives, which may be utilized in the formulations of the invention include, for example, flavoring agents, antimicrobial agents, sweeteners, antioxidants, antistatic agents, lipids such as phospholipids or fatty acids, steroids such as cholesterol, protein excipients such as serum albumin (human serum albumin (HSA), recombinant human albumin (rHA)), gelatin, casein, salt-forming counterions such as sodium and the like. These and additional known pharmaceutical excipients and/or additives suitable for use in the formulations of the invention are known in the art, e.g., as listed in “Remington: The Science & Practice of Pharmacy”, 21^{st} ed., Lippincott Williams & Wilkins, (2005), and in the “Physician's Desk Reference”, 60^{th} ed., Medical Economics, Montvale, N.J. (2005). Pharmaceutically acceptable carriers can be routinely selected that are suitable for the mode of administration, solubility and/or stability of Fc variant protein as well known in the art or as described herein.

It will be understood by one skilled in the art that the formulations of the invention may be isotonic with human blood, that is the formulations of the invention have essentially the same osmotic pressure as human blood. Such isotonic formulations will generally have an osmotic pressure from about 250 mOSm to about 350 mOSm. Isotonicity can be measured by, for example, using a vapor pressure or ice-freezing type osmometer. Tonicity of a formulation is adjusted by the use of tonicity modifiers. “Tonicity modifiers” are those pharmaceutically acceptable inert substances that can be added to the formulation to provide an isotonity of the formulation. Tonicity modifiers suitable for this invention include, but are not limited to, saccharides, salts and amino acids.

In certain embodiments, the formulations of the present invention have an osmotic pressure from about 100 mOSm to about 1200 mOSm, or from about 200 mOSm to about 1000 mOSm, or from about 200 mOSm to about 800 mOSm, or from about 200 mOSm to about 600 mOSm, or from about 250 mOSm to about 500 mOSm, or from about 250 mOSm to about 400 mOSm, or from about 250 mOSm to about 350 mOSm.

In certain embodiments, the formulations of the present invention have an osmotic pressure from 100 mOSm to 1200 mOSm, or from 200 mOSm to 1000 mOSm, or from 200 mOSm to 800 mOSm, or from 200 mOSm to 600 mOSm, or from 250 mOSm to 500 mOSm, or from 250 mOSm to 400 mOSm, or from 250 mOSm to 350 mOSm.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from about 100 moles to about 1000 moles of carbohydrate excipient to about 1 mole of antibody, or from about 200 moles to about 6000 moles of carbohydrate excipient to about 1 mole of antibody, or from about 100 moles to about 510 moles of carbohydrate excipient to about 1 mole of antibody, or from about 100 moles to about 600 moles of carbohydrate excipient to about 1 mole of antibody.

Concentration of any one or any combination of various components of the formulations of the invention are adjusted to achieve the desired tonicity of the final formulation. For example, the ratio of the carbohydrate excipient to antibody may be adjusted according to methods known in the art (e.g., U.S. Pat. No. 6,685,940). In certain embodiments, the molar ratio of the carbohydrate excipient to antibody may be from 100 moles to 1000 moles of carbohydrate excipient to 1 mole of antibody, or from 200 moles to 6000 moles of carbohydrate excipient to 1 mole of antibody, or from 100 moles to 510 moles of carbohydrate excipient to 1 mole of antibody, or from 100 moles to 600 moles of carbohydrate excipient to 1 mole of antibody.

The desired isotonicity of the final formulation may also be achieved by adjusting the salt concentration of the formulations. Salts that are pharmaceutically acceptable and suitable for this invention as tonicity modifiers include, but are not limited to, sodium chloride, sodium succinate, sodium sulfate, potassuim chloride, magnesium chloride, magnesium sulfate, and calcium chloride. In specific embodiments, formulations of the inventions comprise NaCl, MgCl₂, and/or CaCl2. In one embodiment, concentration of NaCl is between about 75 mM and about 150 mM. In another embodiment, concentration of MgCl₂ is between about 1 mM and about 100 mM. Amino acids that are pharmaceutically acceptable and suitable for this invention as tonicity modifiers include, but are not limited to, proline, alanine, L-arginine, asparagine, L-aspartic acid, glycine, serine, lysine, and histidine.

In one embodiment, a formulation of the invention comprises histidine, sodium chloride, trehalose, and Polysorbate 80. In one embodiment, a formulation of the invention comprises sodium chloride, trehalose, and Polysorbate 80. In one embodiment, a formulation of the invention comprises histidine, trehalose, and Polysorbate 80. In one embodiment, a formulation of the invention comprises histidine, sodium chloride, and Polysorbate 80. In one embodiment, a formulation of the invention comprises histidine, sodium chloride, and trehalose. In one embodiment, a formulation of the invention comprises histidine and sodium chloride. In one embodiment, a formulation of the invention comprises histidine and trehalose. In one embodiment, a formulation of the invention comprises histidine and Polysorbate 80. In one embodiment, a formulation of the invention comprises sodium chloride and trehalose. In one embodiment, a formulation of the invention comprises sodium chloride and Polysorbate 80. In one embodiment, a formulation of the invention comprises trehalose, and Polysorbate 80.

In one embodiment, a formulation of the invention comprises histidine, sodium chloride, and Polysorbate 80. In one embodiment, a formulation of the invention comprises between about 5 mM and about 100 mM histidine, between about 10 mM and about 300 mM sodium chloride, between about 0.005% and about 0.1% Polysorbate 80, wherein said formulation has a pH of between about 5.0 and about 7.0. In another embodiment, a formulation of the invention comprises between about 10 mM and about 50 mM histidine, between about 50 mM and about 200 mM sodium chloride, between about 0.01% and about 0.05% Polysorbate 80, wherein said formulation has a pH of between about 5.0 and about 7.0. In a further embodiment, a formulation of the invention comprises about 25 mM histidine, about 125 mM sodium chloride, and about 0.02% Polysorbate 80, wherein said formulation has a pH of between about 5.0 and about 7.0. In a specific embodiment, a formulation of the invention comprises about 25 mM histidine, about 125 mM sodium chloride, and about 0.02% Polysorbate 80, wherein said formulation has a pH of about 5.5. In a specific embodiment, a formulation of the invention comprises about 25 mM histidine, about 125 mM sodium chloride, and about 0.02% Polysorbate 80, wherein said formulation has a pH of about 6.0. In a specific embodiment, a formulation of the invention comprises about 25 mM histidine, about 125 mM sodium chloride, and about 0.02% Polysorbate 80, wherein said formulation has a pH of about 6.5.

In one embodiment, a formulation of the invention comprises histidine, sodium chloride, and Polysorbate 80. In one embodiment, a formulation of the invention comprises between 5 mM and 100 mM histidine, between 10 mM and 300 mM sodium chloride, between 0.005% and 0.1% Polysorbate 80, wherein said formulation has a pH of between 5.0 and 7.0. In another embodiment, a formulation of the invention comprises between 10 mM and 50 mM histidine, between 50 mM and 200 mM sodium chloride, between 0.01% and 0.05% Polysorbate 80, wherein said formulation has a pH of between 5.0 and 7.0. In a further embodiment, a formulation of the invention comprises 25 mM histidine, 125 mM sodium chloride, and 0.02% Polysorbate 80, wherein said formulation has a pH of between 5.0 and 7.0. In a specific embodiment, a formulation of the invention comprises 25 mM histidine, 125 mM sodium chloride, and 0.02% Polysorbate 80, wherein said formulation has a pH of 5.5. In a specific embodiment, a formulation of the invention comprises 25 mM histidine, 125 mM sodium chloride, and 0.02% Polysorbate 80, wherein said formulation has a pH of 6.0. In a specific embodiment, a formulation of the invention comprises 25 mM histidine, 125 mM sodium chloride, and 0.02% Polysorbate 80, wherein said formulation has a pH of 6.5.

In one embodiment, a formulation of the invention consists of between about 20 mg/ml and about 150 mg/ml 13H5 anti-interferon alpha antibody, about 25 mM histidine, about 125 mM sodium chloride, and about 0.02% Polysorbate 80, wherein said formulation has a pH of about 5.5. In one embodiment, a formulation of the invention consists of about 50 mg/ml 13H5 anti-interferon alpha antibody, about 25 mM histidine, about 125 mM sodium chloride, and about 0.02% Polysorbate 80, wherein said formulation has a pH of about 5.5. In one embodiment, a formulation of the invention consists of about 100 mg/ml 13H5 anti-interferon alpha antibody, about 25 mM histidine, about 125 mM sodium chloride, and about 0.02% Polysorbate 80, wherein said formulation has a pH of about 5.5.

In one embodiment, a formulation of the invention consists of between 20 mg/ml and 150 mg/ml 13H5 anti-interferon alpha antibody, 25 mM histidine, 125 mM sodium chloride, and 0.02% Polysorbate 80, wherein said formulation has a pH of 5.5. In one embodiment, a formulation of the invention consists of 50 mg/ml 13H5 anti-interferon alpha antibody, 25 mM histidine, 125 mM sodium chloride, and 0.02% Polysorbate 80, wherein said formulation has a pH of 5.5. In one embodiment, a formulation of the invention consists of 100 mg/ml 13H5 anti-interferon alpha antibody, 25 mM histidine, 125 mM sodium chloride, and 0.02% Polysorbate 80, wherein said formulation has a pH of 5.5.

In one embodiment, a formulation of the invention consists of between about 20 mg/ml and about 150 mg/ml 13H5 anti-interferon alpha antibody, about 25 mM histidine, about 125 mM sodium chloride, and about 0.02% Polysorbate 80, wherein said formulation has a pH of about 6.0. In one embodiment, a formulation of the invention consists of about 50 mg/ml 13H5 anti-interferon alpha antibody, about 25 mM histidine, about 125 mM sodium chloride, and about 0.02% Polysorbate 80, wherein said formulation has a pH of about 6.0. In one embodiment, a formulation of the invention consists of about 100 mg/ml 13H5 anti-interferon alpha antibody, about 25 mM histidine, about 125 mM sodium chloride, and about 0.02% Polysorbate 80, wherein said formulation has a pH of about 6.0.

In one embodiment, a formulation of the invention consists of between 20 mg/ml and 150 mg/ml 13H5 anti-interferon alpha antibody, 25 mM histidine, 125 mM sodium chloride, and 0.02% Polysorbate 80, wherein said formulation has a pH of 6.0. In one embodiment, a formulation of the invention consists of 50 mg/ml 13H5 anti-interferon alpha antibody, 25 mM histidine, 125 mM sodium chloride, and 0.02% Polysorbate 80, wherein said formulation has a pH of 6.0. In one embodiment, a formulation of the invention consists of 100 mg/ml 13H5 anti-interferon alpha antibody, 25 mM histidine, 125 mM sodium chloride, and 0.02% Polysorbate 80, wherein said formulation has a pH of 6.0.

In one embodiment, a formulation of the invention consists of between about 20 mg/ml and about 150 mg/ml 13H5 anti-interferon alpha antibody, about 25 mM histidine, about 125 mM sodium chloride, and about 0.02% Polysorbate 80, wherein said formulation has a pH of about 6.5. In one embodiment, a formulation of the invention consists of about 50 mg/ml 13H5 anti-interferon alpha antibody, about 25 mM histidine, about 125 mM sodium chloride, and about 0.02% Polysorbate 80, wherein said formulation has a pH of about 6.5. In one embodiment, a formulation of the invention consists of about 100 mg/ml 13H5 anti-interferon alpha antibody, about 25 mM histidine, about 125 mM sodium chloride, and about 0.02% Polysorbate 80, wherein said formulation has a pH of about 6.5.

In one embodiment, a formulation of the invention consists of between 20 mg/ml and 150 mg/ml 13H5 anti-interferon alpha antibody, 25 mM histidine, 125 mM sodium chloride, and 0.02% Polysorbate 80, wherein said formulation has a pH of 6.5. In one embodiment, a formulation of the invention consists of 50 mg/ml 13H5 anti-interferon alpha antibody, 25 mM histidine, 125 mM sodium chloride, and 0.02% Polysorbate 80, wherein said formulation has a pH of 6.5. In one embodiment, a formulation of the invention consists of 100 mg/ml 13H5 anti-interferon alpha antibody, 25 mM histidine, 125 mM sodium chloride, and 0.02% Polysorbate 80, wherein said formulation has a pH of 6.5.

In one embodiment, a formulation of the invention comprises histidine, sodium chloride, trehalose and Polysorbate 80. In one embodiment, a formulation of the invention comprises between about 5 mM and about 100 mM histidine, between about 10 mM and about 300 mM sodium chloride, between about 0.3% and about 10% trehalose, and between about 0.005% and about 0.1% Polysorbate 80, wherein said formulation has a pH of between about 5.0 and about 7.0. In another embodiment, a formulation of the invention comprises between about 10 mM and about 50 mM histidine, between about 50 mM and about 200 mM sodium chloride, between about 0.5% and about 5% trehalose, and between about 0.01% and about 0.05% Polysorbate 80, wherein said formulation has a pH of between about 5.5 and about 6.5. In a further embodiment, a formulation of the invention comprises about 25 mM histidine, about 125 mM sodium chloride, about 1.5% trehalose and about 0.02% Polysorbate 80, wherein said formulation has a pH of about 6.0.

In one embodiment, a formulation of the invention comprises histidine, sodium chloride, trehalose and Polysorbate 80. In one embodiment, a formulation of the invention comprises between 5 mM and 100 mM histidine, between 10 mM and 300 mM sodium chloride, between 0.3% and 10% trehalose, and between 0.005% and 0.1% Polysorbate 80, wherein said formulation has a pH of between 5.0 and 7.0. In another embodiment, a formulation of the invention comprises between 10 mM and 50 mM histidine, between 50 mM and 200 mM sodium chloride, between 0.5% and 5% trehalose, and between 0.01% and 0.05% Polysorbate 80, wherein said formulation has a pH of between 5.5 and 6.5. In a further embodiment, a formulation of the invention comprises 25 mM histidine, 125 mM sodium chloride, 1.5% trehalose and 0.02% Polysorbate 80, wherein said formulation has a pH of 6.0.

In one embodiment, a formulation of the invention consists of between about 20 mg/ml and about 150 mg/ml 13H5 anti-interferon alpha antibody, about 25 mM histidine, about 125 mM sodium chloride, about 1.5% trehalose and about 0.02% Polysorbate 80, wherein said formulation has a pH of about 6.0. In another embodiment, a formulation of the invention consists of about 50 mg/ml 13H5 anti-interferon alpha antibody, about 25 mM histidine, about 125 mM sodium chloride, about 1.5% trehalose and about 0.02% Polysorbate 80, wherein said formulation has a pH of about 6.0. In a further embodiment, a formulation of the invention consists of about 100 mg/ml 13H5 anti-interferon alpha antibody, about 25 mM histidine, about 125 mM sodium chloride, about 1.5% trehalose and about 0.02% Polysorbate 80, wherein said formulation has a pH of about 6.0.

In one embodiment, a formulation of the invention consists of between 20 mg/ml and 150 mg/ml 13H5 anti-interferon alpha antibody, 25 mM histidine, 125 mM sodium chloride, 1.5% trehalose and 0.02% Polysorbate 80, wherein said formulation has a pH of 6.0. In another embodiment, a formulation of the invention consists of 50 mg/ml 13H5 anti-interferon alpha antibody, 25 mM histidine, 125 mM sodium chloride, 1.5% trehalose and 0.02% Polysorbate 80, wherein said formulation has a pH of 6.0. In a further embodiment, a formulation of the invention consists of 100 mg/ml 13H5 anti-interferon alpha antibody, 25 mM histidine, 125 mM sodium chloride, 1.5% trehalose and 0.02% Polysorbate 80, wherein said formulation has a pH of 6.0.

In one embodiment, a formulation of the invention comprises histidine, trehalose and Polysorbate 80. In one embodiment, a formulation of the invention comprises between about 5 mM and about 100 mM histidine, between about 1% and about 30% trehalose, and between about 0.005% and about 0.1% Polysorbate 80, wherein said formulation has a pH of between about 5.0 and about 7.0. In another embodiment, a formulation of the invention comprises between about 10 mM and about 50 mM histidine, between about 4% and about 20% trehalose, and between about 0.01% and about 0.05% Polysorbate 80, wherein said formulation has a pH of between about 5.5 and about 6.5. In a further embodiment, a formulation of the invention comprises about 25 mM histidine, about 8% trehalose and about 0.02% Polysorbate 80, wherein said formulation has a pH of about 6.0.

In one embodiment, a formulation of the invention comprises histidine, trehalose and Polysorbate 80. In one embodiment, a formulation of the invention comprises between 5 mM and 100 mM histidine, between 1% and 30% trehalose, and between 0.005% and 0.1% Polysorbate 80, wherein said formulation has a pH of between 5.0 and 7.0. In another embodiment, a formulation of the invention comprises between 10 mM and 50 mM histidine, between 4% and 20% trehalose, and between 0.01% and 0.05% Polysorbate 80, wherein said formulation has a pH of between 5.5 and 6.5. In a further embodiment, a formulation of the invention comprises 25 mM histidine, 8% trehalose and 0.02% Polysorbate 80, wherein said formulation has a pH of 6.0.

In one embodiment, a formulation of the invention consists of between about 20 mg/ml and about 150 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of between about 50 mg/ml and about 120 mg/ml 13H5 anti-interferon alpha antibody, about 25 mM histidine, about 8% trehalose and about 0.02% Polysorbate 80, wherein said formulation has a pH of about 6.0.

In one embodiment, a formulation of the invention consists of between 20 mg/ml and 150 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of between 50 mg/ml and 120 mg/ml 13H5 anti-interferon alpha antibody, 25 mM histidine, 8% trehalose and 0.02% Polysorbate 80, wherein said formulation has a pH of 6.0.

In one embodiment, a formulation of the invention consists of at least about 10 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 20 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 30 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 40 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 50 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 60 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 70 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 80 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 90 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 100 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 110 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of at least about 120 mg/ml 13H5 anti-interferon alpha antibody, about 25 mM histidine, about 8% trehalose and about 0.02% Polysorbate 80, wherein said formulation has a pH of about 6.0.

In one embodiment, a formulation of the invention consists of at least 10 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 20 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 30 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 40 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 50 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 60 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 70 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 80 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 90 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 100 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 110 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of at least 120 mg/ml 13H5 anti-interferon alpha antibody, 25 mM histidine, 8% trehalose and 0.02% Polysorbate 80, wherein said formulation has a pH of 6.0.

In one embodiment, a formulation of the invention consists of about 10 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 20 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 30 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 40 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 50 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 60 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 70 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 80 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 90 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 100 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 110 mg/ml 13H5 anti-interferon alpha antibody, about 25 mM histidine, about 8% trehalose and about 0.02% Polysorbate 80, wherein said formulation has a pH of about 6.0. In one embodiment, a formulation of the invention consists of about 120 mg/ml 13H5 anti-interferon alpha antibody, about 25 mM histidine, about 8% trehalose and about 0.02% Polysorbate 80, wherein said formulation has a pH of about 6.0.

In one embodiment, a formulation of the invention consists of 10 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 20 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 30 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 40 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 50 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 60 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 70 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 80 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 90 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 100 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 110 mg/ml 13H5 anti-interferon alpha antibody, 25 mM histidine, 8% trehalose and 0.02% Polysorbate 80, wherein said formulation has a pH of 6.0. In one embodiment, a formulation of the invention consists of 120 mg/ml 13H5 anti-interferon alpha antibody, 25 mM histidine, 8% trehalose and 0.02% Polysorbate 80, wherein said formulation has a pH of 6.0.

In one embodiment, a formulation of the invention comprises sodium citrate, mannitol, sodium chloride, and Polysorbate 80. In one embodiment, a formulation of the invention comprises mannitol, sodium chloride, and Polysorbate 80. In one embodiment, a formulation of the invention comprises sodium citrate, sodium chloride, and Polysorbate 80. In one embodiment, a formulation of the invention comprises sodium citrate, mannitol, and Polysorbate 80. In one embodiment, a formulation of the invention comprises sodium citrate, mannitol, and sodium chloride. In one embodiment, a formulation of the invention comprises sodium citrate and mannitol. In one embodiment, a formulation of the invention comprises sodium citrate and sodium chloride. In one embodiment, a formulation of the invention comprises sodium citrate and Polysorbate 80. In one embodiment, a formulation of the invention comprises mannitol and sodium chloride. In one embodiment, a formulation of the invention comprises mannitol and Polysorbate 80. In one embodiment, a formulation of the invention comprises sodium chloride and Polysorbate 80.

In one embodiment, a formulation of the invention comprises sodium citrate, mannitol, sodium chloride, and Polysorbate 80. In one embodiment, a formulation of the invention comprises mannitol, sodium chloride, and Polysorbate 80. In one embodiment, a formulation of the invention comprises sodium citrate, sodium chloride, and Polysorbate 80. In one embodiment, a formulation of the invention comprises sodium citrate, mannitol, and Polysorbate 80. In one embodiment, a formulation of the invention comprises sodium citrate, mannitol, and sodium chloride. In one embodiment, a formulation of the invention comprises sodium citrate and mannitol. In one embodiment, a formulation of the invention comprises sodium citrate and sodium chloride. In one embodiment, a formulation of the invention comprises sodium citrate and Polysorbate 80. In one embodiment, a formulation of the invention comprises mannitol and sodium chloride. In one embodiment, a formulation of the invention comprises mannitol and Polysorbate 80. In one embodiment, a formulation of the invention comprises sodium chloride and Polysorbate 80.

In one embodiment, a formulation of the invention comprises sodium citrate, mannitol, sodium chloride, and Polysorbate 80. In one embodiment, a formulation of the invention comprises between about 5 mM and about 100 mM sodium citrate, between about 0.2% and about 6% mannitol, between about 10 mM and about 300 mM sodium chloride, and between about 0.005% and about 0.1% Polysorbate 80, wherein said formulation has a pH of between about 5.0 and about 7.0. In another embodiment, a formulation of the invention comprises between about 10 mM and about 50 mM sodium citrate, between about 0.7% and about 3% mannitol, and between about 0.01% and about 0.05% Polysorbate 80, wherein said formulation has a pH of between about 5.5 and about 6.5. In a further embodiment, a formulation of the invention comprises about 20 mM sodium citrate, about 1.5% mannitol and about 0.02% Polysorbate 80, wherein said formulation has a pH of about 6.0.

In one embodiment, a formulation of the invention comprises sodium citrate, mannitol, sodium chloride, and Polysorbate 80. In one embodiment, a formulation of the invention comprises between 5 mM and 100 mM sodium citrate, between 0.2% and 6% mannitol, between 10 mM and 300 mM sodium chloride, and between 0.005% and 0.1% Polysorbate 80, wherein said formulation has a pH of between 5.0 and 7.0. In another embodiment, a formulation of the invention comprises between 10 mM and 50 mM sodium citrate, between 0.7% and 3% mannitol, and between 0.01% and 0.05% Polysorbate 80, wherein said formulation has a pH of between 5.5 and 6.5. In a further embodiment, a formulation of the invention comprises 20 mM sodium citrate, 1.5% mannitol and 0.02% Polysorbate 80, wherein said formulation has a pH of 6.0.

In one embodiment, a formulation of the invention consists of between about 20 mg/ml and about 150 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of between about 50 mg/ml and about 120 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 20 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 30 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 40 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 50 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 60 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 70 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 80 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 90 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 100 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 110 mg/ml 13H5 anti-interferon alpha antibody, about 20 mM sodium citrate, about 1.5% mannitol and about 0.02% Polysorbate 80, wherein said formulation has a pH of about 6.0. In one embodiment, a formulation of the invention consists of about 120 mg/ml 13H5 anti-interferon alpha antibody, about 20 mM sodium citrate, about 1.5% mannitol and about 0.02% Polysorbate 80, wherein said formulation has a pH of about 6.0.

In one embodiment, a formulation of the invention consists of between 20 mg/ml and 150 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of between 50 mg/ml and 120 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 20 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 30 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 40 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 50 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 60 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 70 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 80 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 90 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 100 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 110 mg/ml 13H5 anti-interferon alpha antibody, 20 mM sodium citrate, 1.5% mannitol and 0.02% Polysorbate 80, wherein said formulation has a pH of 6.0. In one embodiment, a formulation of the invention consists of 120 mg/ml 13H5 anti-interferon alpha antibody, 20 mM sodium citrate, 1.5% mannitol and 0.02% Polysorbate 80, wherein said formulation has a pH of 6.0.

In one embodiment, a formulation of the invention comprises histidine, sucrose, and Polysorbate 80. In one embodiment, a formulation of the invention comprises sucrose and Polysorbate 80. In one embodiment, a formulation of the invention comprises histidine and Polysorbate 80. In one embodiment, a formulation of the invention comprises histidine and sucrose.

In one embodiment, a formulation of the invention comprises histidine, sucrose, and Polysorbate 80. In one embodiment, a formulation of the invention comprises sucrose and Polysorbate 80. In one embodiment, a formulation of the invention comprises histidine and Polysorbate 80. In one embodiment, a formulation of the invention comprises histidine and sucrose.

In one embodiment, a formulation of the invention comprises histidine, sucrose, and Polysorbate 80. In one embodiment, a formulation of the invention comprises between about 5 mM and about 100 mM histidine, between about 2% and about 10% sucrose, and between about 0.005% and about 0.1% Polysorbate 80, wherein said formulation has a pH of between about 5.0 and about 7.0. In another embodiment, a formulation of the invention comprises between about 10 mM and about 50 mM histidine, between about 3% and about 8% sucrose, and between about 0.01% and about 0.05% Polysorbate 80, wherein said formulation has a pH of between about 5.0 and about 7.0. In another embodiment, a formulation of the invention comprises about 25 mM histidine, about 5% sucrose, and about 0.02% Polysorbate 80, wherein said formulation has a pH of about 6.0.

In one embodiment, a formulation of the invention comprises histidine, sucrose, and Polysorbate 80. In one embodiment, a formulation of the invention comprises between 5 mM and 100 mM histidine, between 2% and 10% sucrose, and between 0.005% and 0.1% Polysorbate 80, wherein said formulation has a pH of between 5.0 and 7.0. In another embodiment, a formulation of the invention comprises between 10 mM and 50 mM histidine, between 3% and 8% sucrose, and between 0.01% and 0.05% Polysorbate 80, wherein said formulation has a pH of between 5.0 and 7.0. In another embodiment, a formulation of the invention comprises 25 mM histidine, 5% sucrose, and 0.02% Polysorbate 80, wherein said formulation has a pH of 6.0.

In one embodiment, a formulation of the invention comprises between about 60 mg/ml and about 300 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention comprises about 100 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention comprises about 125 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention comprises about 150 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention comprises about 175 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention comprises about 200 mg/ml 13H5 anti-interferon alpha antibody, about 25 mM histidine, about 5% sucrose, and about 0.02% Polysorbate 80, wherein said formulation has a pH of about 6.0.

In one embodiment, a formulation of the invention comprises between 60 mg/ml and 300 mg/ml 13H5 anti-interferon alpha antibody, 25 mM histidine, 5% sucrose, and 0.02% Polysorbate 80, wherein said formulation has a pH of 6.0. In one embodiment, a formulation of the invention comprises 100 mg/ml 13H5 anti-interferon alpha antibody, 25 mM histidine, 5% sucrose, and 0.02% Polysorbate 80, wherein said formulation has a pH of 6.0. In one embodiment, a formulation of the invention comprises 125 mg/ml 13H5 anti-interferon alpha antibody, 25 mM histidine, 5% sucrose, and 0.02% Polysorbate 80, wherein said formulation has a pH of 6.0. In one embodiment, a formulation of the invention comprises 150 mg/ml 13H5 anti-interferon alpha antibody, 25 mM histidine, 5% sucrose, and 0.02% Polysorbate 80, wherein said formulation has a pH of 6.0. In one embodiment, a formulation of the invention comprises 175 mg/ml 13H5 anti-interferon alpha antibody, 25 mM histidine, 5% sucrose, and 0.02% Polysorbate 80, wherein said formulation has a pH of 6.0. In one embodiment, a formulation of the invention comprises 200 mg/ml 13H5 anti-interferon alpha antibody, 25 mM histidine, 5% sucrose, and 0.02% Polysorbate 80, wherein said formulation has a pH of 6.0.

In one embodiment, a formulation of the invention consists of between about 60 mg/ml and about 300 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention consists of about 100 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention consists of about 125 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention consists of about 150 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention consists of about 175 mg/ml 13H5 anti-interferon alpha antibody, about 25 mM histidine, about 5% sucrose, and about 0.02% Polysorbate 80, wherein said formulation has a pH of about 6.0. In one embodiment, a formulation of the invention consists of about 200 mg/ml 13H5 anti-interferon alpha antibody, about 25 mM histidine, about 5% sucrose, and about 0.02% Polysorbate 80, wherein said formulation has a pH of about 6.0.

In one embodiment, a formulation of the invention consists of between 60 mg/ml and 300 mg/ml 13H5 anti-interferon alpha antibody, 25 mM histidine, 5% sucrose, and 0.02% Polysorbate 80, wherein said formulation has a pH of 6.0. In one embodiment, a formulation of the invention consists of 100 mg/ml 13H5 anti-interferon alpha antibody, 25 mM histidine, 5% sucrose, and 0.02% Polysorbate 80, wherein said formulation has a pH of 6.0. In one embodiment, a formulation of the invention consists of 125 mg/ml 13H5 anti-interferon alpha antibody, 25 mM histidine, 5% sucrose, and 0.02% Polysorbate 80, wherein said formulation has a pH of 6.0. In one embodiment, a formulation of the invention consists of 150 mg/ml 13H5 anti-interferon alpha antibody, 25 mM histidine, 5% sucrose, and 0.02% Polysorbate 80, wherein said formulation has a pH of 6.0. In one embodiment, a formulation of the invention consists of 175 mg/ml 13H5 anti-interferon alpha antibody, 25 mM histidine, 5% sucrose, and 0.02% Polysorbate 80, wherein said formulation has a pH of 6.0. In one embodiment, a formulation of the invention consists of 200 mg/ml 13H5 anti-interferon alpha antibody, 25 mM histidine, 5% sucrose, and 0.02% Polysorbate 80, wherein said formulation has a pH of 6.0.

In one embodiment the formulations of the invention are pyrogen-free formulations which are substantially free of endotoxins and/or related pyrogenic substances. Endotoxins include toxins that are confined inside a microorganism and are released only when the microorganisms are broken down or die. Pyrogenic substances also include fever-inducing, thermostable substances (glycoproteins) from the outer membrane of bacteria and other microorganisms. Both of these substances can cause fever, hypotension and shock if administered to humans. Due to the potential harmful effects, even low amounts of endotoxins must be removed from intravenously administered pharmaceutical drug solutions. The Food & Drug Administration (“FDA”) has set an upper limit of 5 endotoxin units (EU) per dose per kilogram body weight in a single one hour period for intravenous drug applications (The United States Pharmacopeial Convention, Pharmacopeial Forum 26 (1):223 (2000)). When therapeutic proteins are administered in amounts of several hundred or thousand milligrams per kilogram body weight, as can be the case with antibodies, even trace amounts of harmful and dangerous endotoxin must be removed. In certain specific embodiments, the endotoxin and pyrogen levels in the composition are less then 10 EU/mg, or less then 5 EU/mg, or less then 1 EU/mg, or less then 0.1 EU/mg, or less then 0.01 EU/mg, or less then 0.001 EU/mg.

When used for in vivo administration, the formulations of the invention should be sterile. The formulations of the invention may be sterilized by various sterilization methods, including sterile filtration, radiation, etc. In one embodiment, the antibody formulation is filter-sterilized with a presterilized 0.22-micron filter. Sterile compositions for injection can be formulated according to conventional pharmaceutical practice as described in “Remington: The Science & Practice of Pharmacy”, 21^{st} ed., Lippincott Williams & Wilkins, (2005). Formulations comprising antibodies, such as those disclosed herein, ordinarily will be stored in lyophilized form or in solution. It is contemplated that sterile compositions comprising antibodies are placed into a container having a sterile access port, for example, an intravenous solution bag or vial having an adapter that allows retrieval of the formulation, such as a stopper pierceable by a hypodermic injection needle. In one embodiment, a composition of the invention is provided as a pre-filled syringe.

### 5.2. Stability of Formulations

In one embodiment, a formulation of the invention comprises an antibody or fragment thereof that is susceptible to aggregation, fragmentation and/or deamidation.

In one embodiment, a formulation of the invention stabilizes an anti-interferon alpha antibody. In one embodiment, a formulation of the invention prevents aggregation of an anti-interferon alpha antibody or fragment thereof. In another embodiment, a formulation of the invention prevents fragmentation of an anti-interferon alpha antibody or fragment thereof. In a further embodiment, a formulation of the invention prevents deamidation of an anti-interferon alpha antibody or fragment thereof.

In one embodiment, a formulation of the invention stabilizes an anti-interferon alpha antibody. In one embodiment, a formulation of the invention reduces aggregation of an anti-interferon alpha antibody or fragment thereof. In another embodiment, a formulation of the invention reduces fragmentation of an anti-interferon alpha antibody or fragment thereof. In a further embodiment, a formulation of the invention reduces deamidation of an anti-interferon alpha antibody or fragment thereof.

In one embodiment, a formulation of the invention is stable upon storage at about 40° C. for at least about 1 week, at least about 2 weeks, at least about 3 weeks, or at least about 4 weeks. In one embodiment, a formulation of the invention is stable upon storage at about 40° C. for at least about 1 month, at least about 2 months, at least about 3 months, at least about 4 months, at least about 5 months, or at least about 6 months. In a specific embodiment, a formulation of the invention is stable upon storage in a pre-filled syringe.

In one embodiment, a formulation of the invention is stable upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is stable upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stable upon storage in a pre-filled syringe.

In one embodiment, a formulation of the invention is stable upon storage at about 40° C. for about 1 week, about 2 weeks, about 3 weeks, or about 4 weeks. In one embodiment, a formulation of the invention is stable upon storage at about 40° C. for about 1 month, about 2 months, about 3 months, about 4 months, about 5 months, or about 6 months. In a specific embodiment, a formulation of the invention is stable upon storage in a pre-filled syringe.

In one embodiment, a formulation of the invention is stable upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is stable upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stable upon storage in a pre-filled syringe.

In one embodiment, a formulation of the invention comprises an anti-interferon alpha antibody, wherein said antibody retains at least 50% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 50% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 50% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 50% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 50% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 50% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 50% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 50% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 60′% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 60% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 60% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 60% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 60% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 60% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 60% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 60% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 70% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 70% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 70% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 70% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 70% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 70% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 70% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 70% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 80% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 80% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 80% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 80% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 80% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 80% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 80% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 80% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 90% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 90% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 90% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 90% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 90% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 90% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 90% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 90% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 95% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 95% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 95% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 95% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 95% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 95% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 95% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 95% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein said antibody retains at least 99% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises an anti-interferon alpha antibody, wherein said antibody retains at least 99% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein said antibody retains at least 99% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein said antibody retains at least 99% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein said antibody retains at least 99% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises an anti-interferon alpha antibody, wherein said antibody retains at least 99% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein said antibody retains at least 99% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein said antibody retains at least 99% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 50% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 60% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 70% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 80% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 90% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 95% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 40° C. for at least about 1 week, at least about 2 weeks, at least about 3 weeks, or at least about 4 weeks. In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 40° C. for about 1 week, about 2 weeks, about 3 weeks, or about 4 weeks. In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein said antibody retains at least 99% of binding ability to a human interferon alpha polypeptide compared to a reference antibody representing the antibody prior to the storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 1% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 1% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 1% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 1% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 1% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 1% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 1% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 1% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 2% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 2% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 2% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 2% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 2% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 2% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 2% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 2% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 3% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 3% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 3% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 3% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 3% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 3% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 3% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 3% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 4% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 4% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 4% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 4% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 4% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 4% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 4% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 4% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 5% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 5% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 5% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 5% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 5% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 5% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 5% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 5% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 7% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 7% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 7% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 7% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 7% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 7% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 7% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 7% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 10% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 10% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 10% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 10% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 10% of said antibody forms an aggregate as determined by HPSEC upon storage at about 40° C. for about 1 week, about 2 weeks, about 3 weeks, or about 4 weeks. In one embodiment, a formulation of the invention comprises an anti-interferon alpha antibody, wherein less than 10% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 10% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 10% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 1% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 1% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 1% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 1% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 1% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 1% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 1% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 1% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 2% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 2% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 2% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 2% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 2% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 2% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 2% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 2% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 3% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 3% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 3% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 3% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 3% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 3% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 3% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 3% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 4% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 4% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 4% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 4% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 4% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 4% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 4% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 4% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 5% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 5% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 5% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 5% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 5% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 5% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 5% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 5% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 7% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 7% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 7% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 7% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 7% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 7% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 7% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 7% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 10% of said antibody forms an aggregate as determined by HPSEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 10% of said antibody forms an aggregate as determined by HPSEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 10% of said antibody forms an aggregate as determined by HPSEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 10% of said antibody forms an aggregate as determined by HPSEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 10% of said antibody forms an aggregate as determined by HPSEC upon storage at about 40° C. for about 1 week, about 2 weeks, about 3 weeks, or about 4 weeks. In one embodiment, a formulation of the invention comprises 13H5 anti-interferon alpha antibody, wherein less than 10% of said antibody forms an aggregate as determined by HPSEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 10% of said antibody forms an aggregate as determined by HPSEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 10% of said antibody forms an aggregate as determined by HPSEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 1% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 1% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 1% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 1% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 1% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 1% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 1% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 1% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 2% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 2% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 2% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 2% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 2% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 2% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 2% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 2% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 3% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 3% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 3% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 3% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 3% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 3% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 3% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 3% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 4% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 4% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 4% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 4% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 4% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 4% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 4% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 4% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 5% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 5% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 5% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 5% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 5% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 5% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 5% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 5% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 7% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 7% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 7% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 7% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 7% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 7% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 7% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 7% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 10% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 10% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 10′% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 10% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 10% of said antibody is fragmented as determined by RP-HPLC upon storage at about 40° C. for about 1 week, about 2 weeks, about 3 weeks, or about 4 weeks. In one embodiment, a formulation of the invention comprises an anti-interferon alpha antibody, wherein less than 10% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 10% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 10% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 1% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 1% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 1% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 1% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 1% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 1% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 1% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 1% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 2% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 2% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 2% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 2% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 2% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 2% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 2% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 2% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 3% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 3% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 3% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 3% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 3% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 3% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 3% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 3% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 4% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 4% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 4% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 4% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 4% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 4% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 4% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 4% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 5% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 5% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 5% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 5% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 5% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 5% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 5% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 5% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 7% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 7% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 7% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 7% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 7% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 7% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 7% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 7% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 10% of said antibody is fragmented as determined by RP-HPLC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 10% of said antibody is fragmented as determined by RP-HPLC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 10% of said antibody is fragmented as determined by RP-HPLC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 10% of said antibody is fragmented as determined by RP-HPLC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 10% of said antibody is fragmented as determined by RP-HPLC upon storage at about 40° C. for about 1 week, about 2 weeks, about 3 weeks, or about 4 weeks. In one embodiment, a formulation of the invention comprises 13H5 anti-interferon alpha antibody, wherein less than 10% of said antibody is fragmented as determined by RP-HPLC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 10% of said antibody is fragmented as determined by RP-HPLC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 10% of said antibody is fragmented as determined by RP-HPLC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 5% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 5% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 5% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 5% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 5% of said antibody is deamidated as determined by TEC upon storage at about 40° C. for about 1 week, about 2 weeks, about 3 weeks, or about 4 weeks. In one embodiment, a formulation of the invention comprises an anti-interferon alpha antibody, wherein less than 5% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 5% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 5% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 10% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 10% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 10% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 10% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 10% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 10% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 10% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 10% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 20% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 20% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 20% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 20% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 20% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 20% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 20% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 20% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 30% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 30% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 30% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 30% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 30% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 30% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 30% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 30% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 40% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 40% of said antibody is deamidated as determined by TEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 40% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 40% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 40% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 40% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 40′% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 40% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 50% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 50% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 50% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 50% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 50% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 50% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises an anti-interferon alpha antibody, wherein less than 50% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises an anti-interferon alpha antibody, wherein less than 50% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises an anti-interferon alpha antibody, wherein less than 60% of said antibody is deamidated as determined by IEC upon storage at about 40° C. for at least about 1 week, at least about 2 weeks, at least about 3 weeks, or at least about 4 weeks. In one embodiment, a formulation of the invention comprises an anti-interferon alpha antibody, wherein less than 60% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises an anti-interferon alpha antibody, wherein less than 60% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises an anti-interferon alpha antibody, wherein less than 60% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises an anti-interferon alpha antibody, wherein less than 60′% of said antibody is deamidated as determined by IEC upon storage at about 40° C. for about 1 week, about 2 weeks, about 3 weeks, or about 4 weeks. In one embodiment, a formulation of the invention comprises an anti-interferon alpha antibody, wherein less than 60% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 60% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 60% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 5% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 5% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 5% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 5% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 5% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 5% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 5% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 5% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 10% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 10% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 10% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 10% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 10% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 10% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 10% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 10% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 20% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 20% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 20%0 of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 20% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years.

In one embodiment, a formulation of the invention comprises 13H5 anti-interferon alpha antibody, wherein less than 20% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 20% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 20% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 20% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 30% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 30% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 30% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 30% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 30% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 30% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 30% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 30% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 40% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 40% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 40% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 40% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 40% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 40% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 40% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 40% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 50% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 50% of said antibody is deamidated as determined by IEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 50% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 50% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 50% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 50% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 50% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 50′% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention comprises 13H5 anti-interferon alpha antibody, wherein less than 60% of said antibody is deamidated as determined by IEC upon storage at about 40° C. for at least about 1 week, at least about 2 weeks, at least about 3 weeks, or at least about 4 weeks. In one embodiment, a formulation of the invention comprises 13H5 anti-interferon alpha antibody, wherein less than 60% of said antibody is deamidated as determined by TEC upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention comprises 13H5 anti-interferon alpha antibody, wherein less than 60% of said antibody is deamidated as determined by IEC upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention comprises 13H5 anti-interferon alpha antibody, wherein less than 60% of said antibody is deamidated as determined by IEC upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years. In a specific embodiment, a formulation of the invention is stored in a pre-filled syringe.

In one embodiment, a formulation of the invention comprises 13H5 anti-interferon alpha antibody, wherein less than 60% of said antibody is deamidated as determined by IEC upon storage at about 40° C. for about 1 week, about 2 weeks, about 3 weeks, or about 4 weeks. In one embodiment, a formulation of the invention comprises 13H5 anti-interferon alpha antibody, wherein less than 60% of said antibody is deamidated as determined by IEC upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention comprises 13H5 anti-interferon alpha antibody, wherein less than 60% of said antibody is deamidated as determined by IEC upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention comprises 13H5 anti-interferon alpha antibody, wherein less than 60% of said antibody is deamidated as determined by IEC upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In one embodiment, a formulation of the invention is clear and colorless as determined by visual inspection upon storage at about 40° C. for at least about 1 week, at least about 2 weeks, at least about 3 weeks, or at least about 4 weeks. In one embodiment, a formulation of the invention is clear and colorless as determined by visual inspection upon storage at about 40° C. for at least about 1 month, at least about 2 months, at least about 3 months, at least about 4 months, at least about 5 months, or at least about 6 months. In a specific embodiment, a formulation of the invention is stored in a pre-filled syringe.

In one embodiment, a formulation of the invention is clear and colorless as determined by visual inspection upon storage at about 5° C. for at least about 1 month, at least about 2 months, at least about 3 months, at least about 4 months, at least about 5 months, at least about 6 months, at least about 7 months, at least about 8 months, at least about 9 months, at least about 10 months, at least about 11 months, or at least about 12 months. In one embodiment, a formulation of the invention is clear and colorless as determined by visual inspection upon storage at about 5° C. for at least about 1 year, at least about 2 years, at least about 3 years, at least about 4 years, at least about 5 years, at least about 6 years, at least about 7 years, at least about 8 years, at least about 9 years, at least about 10 years, at least about 11 years, or at least about 12 years.

In one embodiment, a formulation of the invention is clear and colorless as determined by visual inspection upon storage at about 40° C. for about 1 week, about 2 wccks, about 3 weeks, or about 4 weeks. In one embodiment, a formulation of the invention is clear and colorless as determined by visual inspection upon storage at about 40° C. for about 1 month, about 2 months, about 3 months, about 4 months, about 5 months, or about 6 months. In a specific embodiment, a formulation of the invention is stored in a pre-filled syringe.

In one embodiment, a formulation of the invention is clear and colorless as determined by visual inspection upon storage at about 5° C. for about 1 month, about 2 months, about 3 months, about 4 months, about 5 months, about 6 months, about 7 months, about 8 months, about 9 months, about 10 months, about 11 months, or about 12 months. In one embodiment, a formulation of the invention is clear and colorless as determined by visual inspection upon storage at about 5° C. for about 1 year, about 2 years, about 3 years, about 4 years, about 5 years, about 6 years, about 7 years, about 8 years, about 9 years, about 10 years, about 11 years, or about 12 years. In a specific embodiment, a formulation of the invention is stored in a pre-filled syringe.

In certain embodiments, the formulations of the invention maintain improved aggregation profiles upon storage, for example, for extended periods (for example, but not limited to 1 week, 1 month, 6 months, 1 year, 2 years, 3 years or 5 years) at room temperature or 4° C. or for periods (such as, but not limited to 1 week, 2 weeks, 3 weeks, 1 month, 2 months, 3 months, or 6 months) at elevated temperatures such as 38° C.-42° C. In certain embodiments, the formulations maintain improved aggregation profiles upon storage while exposed to light or stored in the dark in a variety of humidity conditions including but not limited to a relative humidity of up to 10%, or up to 20%, or up to 30%, or up to 40%, or up to 50%, or up to 60%, or up to 70%, or up to 80%, or up to 90%, or up to 100%. It will be understood in the art that the term “ambient” conditions generally refers to temperatures of about 20° C. at a relative humidity of between 10% and 60% with exposure to light. Similarly, temperatures between about 2° C. and about 8° C. at a relative humidity of less then about 10% are collectively referred to as “4° C.” or “5° C.”, temperatures between about 23° C. and about 27° C. at a relative humidity of about 60% are collectively referred to as “25° C.” and temperatures between about 38° C. and about 42° C. at a relative humidity of about 75% are collectively referred to as “40° C.” In a specific embodiment, a formulation of the invention is stored in a pre-filled syringe.

In certain embodiments, after storage at 4° C. for at least one month, the formulations of the invention comprise (or consists of as the aggregate fraction) a particle profile of less than about 3.4 E+5 particles/ml of diameter 2-4 μm, less than about 4.0 E+4 particles/ml of diameter 4-10 μm, less than about 4.2 E+3 particles/ml of diameter 10-20 μm, less than about 5.0 E+2 particles/ml of diameter 20-30 μm, less than about 7.5 E+1 particles/ml of diameter 30-40 μm, and less than about 9.4 particles/ml of diameter 40-60 μm as determined by a particle multisizer. In certain embodiments, the formulations of the invention contain no detectable particles greater than 40 μm, or greater than 30 μm. In a specific embodiment, a formulation of the invention is stored in a pre-filled syringe.

Numerous methods useful for determining the degree of aggregation, and/or types and/or sizes of aggregates present in a protein formulation (e.g., antibody formulation of the invention) are known in the art, including but not limited to, size exclusion chromatography (SEC), high performance size exclusion chromatography (HPSEC), static light scattering (SLS), Fourier Transform Infrared Spectroscopy (FTIR), circular dichroism (CD), urea-induced protein unfolding techniques, intrinsic tryptophan fluorescence, differential scanning calorimetry, and 1-anilino-8-naphthalenesulfonic acid (ANS) protein binding techniques. For example, size exclusion chromatography (SEC) may be performed to separate molecules on the basis of their size, by passing the molecules over a column packed with the appropriate resin, the larger molecules (e.g. aggregates) will elute before smaller molecules (e.g. monomers). The molecules are generally detected by UV absorbance at 280 nm and may be collected for further characterization. High pressure liquid chromatographic columns are often utilized for SEC analysis (HP-SEC). Specific SEC methods are detailed in the section entitled “Examples” infra. Alternatively, analytical ultracentrifugation (AUC) may be utilized. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Protein aggregation in the formulations may also be characterized by particle counter analysis using a coulter counter or by turbidity measurements using a turbidimeter. Turbidity is a measure of the amount by which the particles in a solution scatter light and, thus, may be used as a general indicator of protein aggregation. In addition, non-reducing polyacrylamide gel electrophoresis (PAGE) or capillary gel electrophoresis (CGE) may be used to characterize the aggregation and/or fragmentation state of antibodies or a fragment thereof in a formulation of the invention.

In one embodiment, a formulation of the invention is for parenteral administration. In one embodiment, a formulation of the invention is an injectable formulation. In one embodiment, a formulation of the invention is for intravenous, subcutaneous, or intramuscular administration. In a specific embodiment, a formulation of the invention comprises 13H5 anti-interferon alpha antibody wherein said formulation is for subcutaneous injection.

In one embodiment, a formulation of the invention is for intravenous administration wherein said formulation comprises between about 20 mg/ml and about 40 mg/ml of an anti-interferon alpha antibody or a fragment thereof. In a specific embodiment, a formulation of the invention is for intravenous administration wherein said formulation comprises between about 20 mg/ml and about 40 mg/ml 13H5 anti-interferon alpha antibody.

In one embodiment, a formulation of the invention is for subcutaneous administration wherein said formulation comprises between about 70 mg/ml and about 250 mg/ml of an anti-interferon alpha antibody or a fragment thereof. In a specific embodiment, a formulation of the invention is for subcutaneous administration wherein said formulation comprises between about 70 mg/ml and about 250 mg/ml 13H5 anti-interferon alpha antibody.

In one embodiment, a formulation of the invention is for aerosol administration.

The present invention also provides a pharmaceutical unit dosage form suitable for parenteral administration to a human which comprises an anti-interferon alpha antibody formulation in a suitable container. In one embodiment, a pharmaceutical unit dosage of the invention comprises 13H5 anti-interferon alpha antibody. In one embodiment, a pharmaceutical unit dosage of the invention comprises an intravenously, subcutaneously, or intramuscularly delivered anti-interferon alpha antibody formulation. In another embodiment, a pharmaceutical unit dosage of the invention comprises aerosol delivered anti-interferon alpha antibody formulation. In a specific embodiment, a pharmaceutical unit dosage of the invention comprises a subcutaneously delivered 13H5 anti-interferon alpha antibody formulation. In another embodiment, a pharmaceutical unit dosage of the invention comprises an aerosol delivered anti-interferon alpha antibody formulation. In a further embodiment, a pharmaceutical unit dosage of the invention comprises an intranasally administered anti-interferon alpha antibody formulation. In one embodiment, a suitable container is a pre-filled syringe.

In one embodiment, a formulation of the invention is provided in a sealed container.

The present invention further provided a kit comprising an anti-interferon alpha antibody formulation of the invention.

The present invention also provides methods of preventing, managing, treating or ameliorating an inflammatory disease or disorder, an autoimmune disease or disorder, a proliferative disease, an infection, a disease or disorder associated with or characterized by aberrant expression and/or activity of an interferon alpha polypeptide, a disease or disorder associated with or characterized by aberrant expression and/or activity of the interferon alpha receptor or one or more subunits thereof, or one or more symptoms thereof.

In one embodiment, a method of the invention comprises administering to a subject in need thereof a prophylactically or therapeutically effective amount of an anti-interferon alpha antibody formulation. In a specific embodiment, a method of the invention comprises administering to a subject in need thereof a prophylactically or therapeutically effective amount of a 13H5 anti-interferon alpha antibody formulation.

In one embodiment, a method of the invention is for the prevention, treatment, management or amelioration of a disease or disorder selected from the group consisting of multiple sclerosis, inflammatory bowel disease, insulin dependent diabetes mellitus, psoriasis, autoimmune thyroiditis, rheumatoid arthritis, glomerulonephritis, systemic lupus erythematosus, idiopathic inflammatory myopathies (IIM), dermatomyositis (DM), polymyositis (PM), and inclusion body myositis (IBM). In a specific embodiment, a method of the invention is for the prevention, treatment, management or amelioration of systemic lupus erythematosus. In another embodiment, a method of the invention is for the prevention, treatment, management or amelioration of transplant rejection or graft versus host disease. In a further embodiment, a method of the invention is for the prevention, treatment, management or amelioration of idiopathic inflammatory myopathies (IIM), dermatomyositis (DM), polymyositis (PM), and inclusion body myositis (IBM).

In one embodiment, a method of the invention for the prevention, treatment, management or amelioration of a disease or disorder further comprises administering to said subject a prophylactically or therapeutically effective amount of a prophylactic or therapeutic agent other than an antibody or antibody fragment that specifically binds to an interferon alpha polypeptide.

In one embodiment, a method of the invention for the prevention, treatment, management or amelioration of a disease or disorder further comprises administering to said subject a prophylactically or therapeutically effective amount of a prophylactic or therapeutic agent other than an antibody or antibody fragment that specifically binds to an interferon alpha polypeptide, wherein said prophylactic or therapeutic agent is an anti-inflammatory agent, immunomodulatory agent, anti-angiogenic agent, or anti-cancer agent.

### 5.3. Antibodies Useful in the Formulations of the Invention

The present invention provides formulations comprising monoclonal antibodies that bind to IFN alpha and inhibit the biological activity of multiple IFN alpha subtypes. In certain embodiments, the antibodies of the invention are capable of inhibiting surface expression of cell markers induced by IFN alpha, inhibiting IP-10 expression induced by IFN alpha and/or inhibiting dendritic cell development mediated by plasma from patients with systemic lupus erythematosus (SLE). These antibodies can be used for therapeutic, including prophylactic, purposes, for example in situations where the production or expression of interferon alpha is associated with pathological symptoms. Such antibodies can also be used for the diagnosis of various diseases or for the study of the evolution of such diseases.

The antibodies useful in the present invention include, but are not limited to, monoclonal antibodies, synthetic antibodies, multispecific antibodies (including bi-specific antibodies), human antibodies, humanized antibodies, chimeric antibodies, single-chain Fvs (scFv) (including bi-specific scFvs), single chain antibodies, Fab fragments, F(ab′) fragments, disulfide-linked Fvs (sdFv), and epitope-binding fragments of any of the above. In particular, antibodies of the present invention include immunoglobulin molecules and immunologically active portions of immunoglobulin molecules, i.e., molecules that contain an antigen binding site that specifically binds to an antigen. The immunoglobulin molecules of the invention can be of any type (e.g., IgG, IgE, IgM, IgD, IgA and IgY), class (e.g., IgG₁, IgG₂, IgG₃, IgG₄, IgA₁ and IgA₂) or subclass of immunoglobulin molecule.

The antibodies useful in the present invention may be from any animal origin including birds and mammals (for example, but not limited to, human, murine, donkey, sheep, rabbit, goat, guinea pig, camel, horse, or chicken). In specific embodiments, the antibodies are human or humanized monoclonal antibodies.

The antibodies useful in the present invention may be monospecific, bispecific, trispecific or of greater multispecificity. Multispecific antibodies may specifically bind to different epitopes of a polypeptide or may specifically bind to both a polypeptide as well a heterologous epitope, such as a heterologous polypeptide or solid support material. See, e.g., International Publication Nos. WO 93/17715, WO 92/08802, WO 91/00360, and WO 92/05793; Tutt, et al., 1991, J. Immunol. 147:60-69; U.S. Pat. Nos. 4,474,893, 4,714,681, 4,925,648, 5,573,920, and 5,601,819; and Kostelny et al., 1992, J. Immunol. 148:1547-1553.

The antibodies useful in the present invention can be single-chain antibodies. The design and construction of a single-chain antibody is described in Marasco et al, 1993, Proc Natl Acad Sci 90:7889-7893, which is incorporated herein by reference in its entirety.

In specific embodiments, the present invention provides formulations of antibodies that specifically bind to an interferon alpha polypeptide (e.g., a human interferon alpha polypeptide). In specific embodiments, the invention provides for the formulations of the following antibodies that specifically bind to an interferon alpha polypeptide: 13H5 or an antigen-binding fragment thereof, 13H7 or an antigen binding fragment thereof, 7H9 or and antigenbinding fragment thereof (see, US Patent Publication 2007/0014724A1).

The present invention provides formulations of antibodies that specifically bind an interferon alpha polypeptide, said antibodies comprising a VH domain having an amino acid sequence of the VH domain of 13H5 (SEQ ID NO:2), 13H7 (SEQ ID NO: 11), or 7H9 (SEQ ID NO:19). In a specific embodiment, an antibody that specifically binds to an interferon alpha polypeptide comprises a VH domain having an amino acid sequence of SEQ ID NO:2.

The present invention provides formulations of antibodies that specifically bind to an interferon alpha polypeptide, said antibodies comprising a VH CDR selected from the group comprising SEQ ID NO: 3-5, 12-14, and 20-22. In particular, the invention provides antibodies that specifically bind to an interferon alpha polypeptide, said antibodies comprising one, two, three, four, five or more VH CDRs selected from the group comprising SEQ ID NO: 3-5, 12-14, and 20-22. In one embodiment, an antibody that specifically binds to an interferon alpha polypeptide comprises a VH CDR1 having the amino acid sequence of SEQ ID NO.:3, 12 or20. In another embodiment, an antibody that specifically binds to an interferon alpha polypeptide comprises a VH CDR2 having the amino acid sequence of SEQ ID NO.:4, 13 or 21. In another embodiment, an antibody that specifically binds to an interferon alpha polypeptide comprises a VH CDR3 having the amino acid sequence of SEQ ID NO.:5, 14 or 22. In another embodiment, an antibody that specifically binds to an interferon alpha polypeptide may comprise a VH CDR1 having the amino acid sequence of SEQ ID NO.:3, 12 or 20; a VH CDR2 having the amino acid sequence of SEQ ID NO.:4, 13 or 21; and may further comprise a VH CDR3 having the amino acid sequence of SEQ ID NO.:5, 14 or 22. In a further embodiment, an antibody that specifically binds to an interferon alpha polypeptide comprises a VH CDR1 having the amino acid sequence of SEQ ID NO.:3; a VH CDR2 having the amino acid sequence of SEQ ID NO.:4; and a VH CDR3 having the amino acid sequence of SEQ ID NO.:5. In another embodiment, an antibody that specifically binds to an interferon alpha polypeptide comprises a VH CDR1 having the amino acid sequence of SEQ ID NO.:12; a VH CDR2 having the amino acid sequence of SEQ ID NO.13; and a VH CDR3 having the amino acid sequence of SEQ ID NO. 14. In another embodiment, an antibody that specifically binds to an interferon alpha polypeptide comprises a VH CDR1 having the amino acid sequence of SEQ ID NO.:20; a VH CDR2 having the amino acid sequence of SEQ ID NO. 21; and a VH CDR3 having the amino acid sequence of SEQ ID NO. 22.

The present invention provides formulations of antibodies that specifically bind to an interferon alpha polypeptide, said antibodies comprising a VL domain having an amino acid sequence of the VL domain of 13H5 (SEQ ID NO:7), 13H7 (SEQ ID NO:15), or 7H9 (SEQ ID NO:23). In a specific embodiment, an antibody that specifically binds to an interferon alpha polypeptide comprises a VL domain having an amino acid sequence of SEQ ID NO:7.

The present invention provides formulations of antibodies that specifically bind to an interferon alpha polypeptide, said antibodies comprising a VL CDR selected from the group comprising SEQ ID NO: 8-10, 16-18, and 24-26. In particular, the invention provides antibodies that specifically bind to an interferon alpha polypeptide, said antibodies comprising one, two, three, four, five or more VL CDRs selected from the group comprising SEQ ID NO: 8-10, 16-18, and 24-26. In one embodiment, an antibody that specifically binds to an interferon alpha polypeptide comprises a VL CDR1 having the amino acid sequence of SEQ ID NO.:8, 16 or 24. In another embodiment, an antibody that specifically binds to an interferon alpha polypeptide comprises a VL CDR2 having the amino acid sequence of SEQ ID NO.:9, 17 or 25. In another embodiment, an antibody that specifically binds to an interferon alpha polypeptide comprises a VL CDR3 having the amino acid sequence of SEQ ID NO.:10, 18 or 26. In another embodiment, an antibody that specifically binds to an interferon alpha polypeptide may comprise a VL CDR1 having the amino acid sequence of SEQ ID NO. 8, 16 or 24; a VL CDR2 having the amino acid sequence of SEQ ID NO. 9, 17 or 25; and may further comprise a VL CDR3 having the amino acid sequence of SEQ ID NO. 10, 18 or 26. In a further embodiment, an antibody that specifically binds to an interferon alpha polypeptide comprises a VL CDR1 having the amino acid sequence of SEQ ID NO.:8; a VL CDR2 having the amino acid sequence of SEQ ID NO.9; and a VL CDR3 having the amino acid sequence of SEQ ID NO. 10. In another embodiment, an antibody that specifically binds to an interferon alpha polypeptide comprises a VL CDR1 having the amino acid sequence of SEQ ID NO.:16; a VL CDR2 having the amino acid sequence of SEQ ID NO.:17; and a VL CDR3 having the amino acid sequence of SEQ ID NO.18. In another embodiment, an antibody that specifically binds to an interferon alpha polypeptide comprises a VL CDR1 having the amino acid sequence of SEQ ID NO.:24; a VL CDR2 having the amino acid sequence of SEQ ID NO. 25; and a VL CDR3 having the amino acid sequence of SEQ ID NO. 26.

The present invention provides formulations of antibodies that specifically bind to an interferon alpha polypeptide, said antibodies may comprise a VH CDR selected from the group comprising SEQ ID NO: 3-5, 12-14, and 20-22 and a VL CDR selected from the group comprising SEQ ID NO: 8-10, 16-18, and 24-26. In one embodiment, the invention provides antibodies that specifically bind to an interferon alpha polypeptide, wherein said antibodies may comprises one, two, three, four, five or more VH CDRs selected from the group comprising SEQ ID NO: 3-5, 12-14, and 20-22, and may further comprise one, two, three, four, five or more VL CDRs selected from the group comprising SEQ ID NO: 8-10, 16-18, and 24-26.

The present invention provides formulations of antibodies that specifically bind to an interferon alpha polypeptide, said antibodies may comprise a VH domain having an amino acid sequence of SEQ ID NO:2, 11 or 19; and a VL domain having an amino acid sequence of SEQ ID NO:7, 15 or 23. In a specific embodiment, an antibody that specifically binds to an interferon alpha polypeptide comprises a VH domain having an amino acid sequence of SEQ ID NO:2 and a VH domain having an amino acid sequence of SEQ ID NO:7. In a specific embodiment, an antibody that specifically binds to an interferon alpha polypeptide comprises a VH domain having an amino acid sequence of SEQ ID NO:11 and a VH domain having an amino acid sequence of SEQ ID NO: 15. In a specific embodiment, an antibody that specifically binds to an interferon alpha polypeptide comprises a VH domain having an amino acid sequence of SEQ ID NO:19 and a VH domain having an amino acid sequence of SEQ ID NO:23.

The present invention provides formulations of antibodies that specifically bind to an interferon alpha polypeptide, said antibodies comprising derivatives of the VH domains, VH CDRs, VL domains, or VL CDRs described herein that specifically bind to an interferon alpha polypeptide. Standard techniques known to those of skill in the art can be used to introduce mutations (e.g., deletions, additions, and/or substitutions) in the nucleotide sequence encoding an antibody of the invention, including, for example, site-directed mutagenesis and PCR-mediated mutagenesis which results in amino acid substitutions. In one embodiment, the derivatives include less than 25 amino acid substitutions, less than 20 amino acid substitutions, less than 15 amino acid substitutions, less than 10 amino acid substitutions, less than 5 amino acid substitutions, less than 4 amino acid substitutions, less than 3 amino acid substitutions, or less than 2 amino acid substitutions relative to the original molecule. In one embodiment, the derivatives have conservative amino acid substitutions are made at one or more predicted non-essential amino acid residues (i.e., amino acid residues which are not critical for the antibody to specifically bind to an interferon alpha polypeptide). A “conservative amino acid substitution” is one in which the amino acid residue is replaced with an amino acid residue having a side chain with a similar charge. Families of amino acid residues having side chains with similar charges have been defined in the art. These families include amino acids with basic side chains (for example, but not limited to, lysine, arginine, histidine), acidic side chains (for example, but not limited to, aspartic acid, glutamic acid), uncharged polar side chains (for example, but not limited to, glycine, asparagine, glutamine, serine, threonine, tyrosine, cysteine), nonpolar side chains (for example, but not limited to, alanine, valine, leucine, isoleucine, proline, phenylalanine, methionine, tryptophan), beta-branched side chains (for example, but not limited to, threonine, valine, isoleucine) and aromatic side chains (for example, but not limited to, tyrosine, phenylalanine, tryptophan, histidine). Alternatively, mutations can be introduced randomly along all or part of the coding sequence, such as by saturation mutagenesis, and the resultant mutants can be screened for biological activity to identify mutants that retain activity. Following mutagenesis, the encoded antibody can be expressed and the activity of the antibody can be determined.

In specific embodiments, the present invention provides for formulations of antibodies that specifically bind to an interferon alpha polypeptide, said antibodies comprising the amino acid sequence of 13H5, 13H7 or 7H9 with one or more amino acid residue substitutions in the variable light (VL) domain and/or variable heavy (VH) domain. The present invention also provides for antibodies that specifically bind to an interferon alpha polypeptide, said antibodies comprising the amino acid sequence of 13H5, 13H7 or 7H9 with one or more amino acid residue substitutions in one or more VL CDRs and/or one or more VH CDRs. The present invention also provides for antibodies that specifically bind to an interferon alpha polypeptide, said antibodies comprising the amino acid sequence of 13H5, 13H7 or 7H9, or a VH and/or VL domain thereof with one or more amino acid residue substitutions in one or more VH frameworks and/or one or more VL frameworks. The antibody generated by introducing substitutions in the VH domain, VH CDRs, VL domain VL CDRs and/or frameworks of 13H5, 13H7 or 7H9 can be tested in vitro and/or in vivo, for example, for its ability to bind to an interferon alpha polypeptide, or for its ability to inhibit or reduce interferon alpha mediated cell proliferation, or for its ability to prevent, treat and/or manage an autoimmune disorder, an inflammatory disorder or a proliferative disorder, or a symptom thereof.

In a specific embodiment, an antibody that specifically binds to an interferon alpha polypeptide comprises an amino acid sequence that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the amino acid sequence of 13H5, 13H7 or 7H9, or an antigen-binding fragment thereof. In another embodiment, an antibody that specifically binds to an interferon alpha polypeptide comprises an amino acid sequence of a VH domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VH domain of 13H5, 13H7 or 7H9. In another embodiment, an antibody that specifically binds to an interferon alpha polypeptide comprises an amino acid sequence of a VL domain that is at least 35%, at least 40%, at least 45%, at least 50%, at least 55%, at least 60%, at least 65%, at least 70%, at least 75%, at least 80%, at least 85%, at least 90%, at least 95%, or at least 99% identical to the VL domain of 13H5, 13H7 or 7H9.

In another embodiment, an antibody that specifically binds to an interferon alpha polypeptide comprises an amino acid sequence of one or more VL CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any VL CDRs of 13H5, 13H7 or 7H9. In another embodiment, an antibody that specifically binds to an interferon alpha polypeptide comprises an amino acid sequence of one or more VH CDRs that are at least 35%, at least 40%, at least 45%, at least 50%, at least 55%, at least 60%, at least 65%, at least 70%, at least 75%, at least 80%, at least 85%, at least 90%, at least 95%, or at least 99% identical to any VH CDRs of 13H5, 13H7 or 7H9.

The present invention encompasses formulations of antibodies that compete with an antibody described herein for binding to an interferon alpha polypeptide. In particular, the present invention encompasses antibodies that compete with 13H5, 13H7 or 7H9, or an antigen-binding fragment thereof for binding to the interferon alpha polypeptide.

The present invention encompasses formulations of polypeptides or proteins comprising (alternatively, consisting of) VH domains that compete with the VH domain of 13H5, 13H7 or 7H9 for binding to an interferon alpha polypeptide. The present invention also encompasses formulations of polypeptides or proteins comprising (alternatively, consisting of) VL domains that compete with a VL domain of 13H5, 13H7 or 7H9 for binding to an interferon alpha polypeptide.

The antibodies that specifically bind to an interferon alpha polypeptide include derivatives that are modified, i.e., by the covalent attachment of any type of molecule to the antibody such that covalent attachment does not eliminate binding to an interferon alpha polypeptide. For example, but not by way of limitation, the antibody derivatives include antibodies that have been modified, for example, but not limited to, by glycosylation, acetylation, pegylation, phosphorylation, amidation, derivatization by known protecting/blocking groups, proteolytic cleavage, linkage to a cellular ligand or other protein, etc. Any of numerous chemical modifications may be carried out by known techniques, including, but not limited to, specific chemical cleavage, acetylation, formylation, metabolic synthesis of tunicamycin, etc. Additionally, the derivative may contain one or more non-classical amino acids.

The invention encompasses formulations of antibodies that specifically bind to an interferon alpha polypeptide found in the milieu, i.e., not bound to an interferon alpha receptor or a subunit thereof. The invention also encompasses antibodies that specifically bind to an interferon alpha polypeptide bound to a soluble interferon alpha receptor subunit. The invention further encompasses antibodies that specifically bind to an interferon alpha polypeptide bound to a cellular membrane-bound interferon alpha receptor or a subunit thereof.

The formulations of antibodies of the present invention that specifically bind to an interferon alpha polypeptide may be monospecific, bispecific, trispecific or of greater multispecificity. Multispecific antibodies may be specific for different epitopes of an interferon alpha polypeptide or may be specific for both an interferon alpha polypeptide as well as for a heterologous epitope, such as a heterologous polypeptide or solid support material. See, e.g., International publications WO 93/17715, WO 92/08802, WO 91/00360, and WO 92/05793; Tutt, et al., J. Immunol. 147:60-69(1991); U.S. Pat. Nos. 4,474,893, 4,714,681, 4,925,648, 5,573,920, and 5,601,819; and Kostelny et al., J. Immunol. 148:1547-1553 (1992).

### 5.3.1. Antibodies Having Increased Half-Lives

The present invention provides for formulations of antibodies and antibody fragments that specifically bind to an antigen of interest (e.g., an interferon alpha polypeptide) which have an extended half-life in vivo. In particular, the present invention provides formulations of antibodies and antibody fragments that specifically bind to an antigen of interest (e.g., an interferon alpha polypeptide) which have a half-life in a mammal (for example, but not limited to, a human), of greater than 3 days, greater than 7 days, greater than 10 days, greater than 15 days, greater than 25 days, greater than 30 days, greater than 35 days, greater than 40 days, greater than 45 days, greater than 2 months, greater than 3 months, greater than 4 months, or greater than 5 months.

To prolong the serum circulation of antibodies (for example, but not limited to, monoclonal antibodies and single chain antibodies) or antibody fragments (for example, but not limited to, Fab fragments) in vivo, for example, inert polymer molecules such as high molecular weight polyethyleneglycol (PEG) can be attached to the antibodies (including antibody fragments thereof) with or without a multifunctional linker either through site-specific conjugation of the PEG to the N- or C-terminus of the antibodies or via epsilon-amino groups present on lysine residues. Linear or branched polymer derivatization that results in minimal loss of biological activity will be used. The degree of conjugation can be closely monitored by SDS-PAGE and mass spectrometry to ensure proper conjugation of PEG molecules to the antibodies. Unreacted PEG can be separated from antibody-PEG conjugates by size-exclusion or by ion-exchange chromatography. PEG-derivatized antibodies (including antibody fragments thereof) can be tested for binding activity as well as for in vivo efficacy using methods known to those of skill in the art, for example, by immunoassays described herein.

Antibodies having an increased half-life in vivo can also be generated introducing one or more amino acid modifications (i.e., substitutions, insertions or deletions) into an IgG constant domain, or FcRn binding fragment thereof (e.g., Fc or hinge-Fc domain fragment). See, e.g., International Publication No. WO 98/23289; International Publication No. WO 97/34631; and U.S. Pat. No. 6,277,375, each of which is incorporated herein by reference in its entirety.

Further, antibodies (including antibody fragments thereof) can be conjugated to albumin in order to make the antibody (including antibody fragment thereof) more stable in vivo or have a longer half life in vivo. The techniques are well known in the art, see e.g., International Publication Nos. WO 93/15199, WO 93/15200, and WO 01/77137; and European Patent No. EP 413, 622, all of which are incorporated herein by reference.

### 5.3.2. Antibody Conjugates

The present invention provides formulations of antibodies (including antibody fragments thereof) that specifically binds to an antigen of interest (e.g., an interferon alpha polypeptide) recombinantly fused or chemically conjugated (including both covalent and non-covalent conjugations) to a heterologous protein or polypeptide (or fragment of a polypeptide of at least 10, at least 20, at least 30, at least 40, at least 50, at least 60, at least 70, at least 80, at least 90 or at least 100 amino acids) to generate fusion proteins. In particular, the invention provides formulations of fusion proteins comprising an antigen-binding fragment of an antibody described herein (for example, but not limited to, a Fab fragment, Fd fragment, Fv fragment, F(ab)₂ fragment, a VH domain, a VH CDR, a VL domain or a VL CDR) and a heterologous protein, polypeptide, or peptide. Methods for fusing or conjugating proteins, polypeptides, or peptides to an antibody (including antibody fragment thereof) are known in the art. See, e.g., U.S. Pat. Nos. 5,336,603, 5,622,929, 5,359,046, 5,349,053, 5,447,851, and 5,112,946; European Patent Nos. EP 307,434 and EP 367,166; International Publication Nos. WO 96/04388 and WO 91/06570; Ashkenazi et al., 1991, Proc. Natl. Acad. Sci. USA 88: 10535-10539; Zheng et al., 1995, J. Immunol. 154:5590-5600; and Vil et al., 1992, Proc. Natl. Acad. Sci. USA 89:11337-11341 (said references are incorporated herein by reference in their entireties).

Additional fusion proteins may be generated through the techniques of gene-shuffling, motif-shuffling, exon-shuffling, and/or codon-shuffling (collectively referred to as “DNA shuffling”). DNA shuffling may be employed to alter the activities of antibodies of the invention or fragments thereof (for example, but not limited to, antibodies or fragments thereof with higher affinities and lower dissociation rates). See, generally, U.S. Pat. Nos. 5,605,793, 5,811,238, 5,830,721, 5,834,252, and 5,837,458; Patten et al., 1997, Curr. Opinion Biotechnol. 8:724-33; Harayama, 1998, Trends Biotechnol. 16(2):76-82; Hansson, et al., 1999, J. Mol. Biol. 287:265-76; and Lorenzo and Blasco, 1998, Biotechniques 24(2):308-313 (each of these patents and publications are hereby incorporated by reference in its entirety). Antibodies (including antibody fragments thereof), or the encoded antibodies or fragments thereof, may be altered by being subjected to random mutagenesis by error-prone PCR, random nucleotide insertion or other methods prior to recombination. A polynucleotide encoding an antibody (including antibody fragment thereof) thereof may be recombined with one or more components, motifs, sections, parts, domains, fragments, etc. of one or more heterologous molecules.

Moreover, the antibodies (including antibody fragments thereof) can be fused to marker sequences, such as a peptide to facilitate purification. The marker amino acid sequence may be a hexa-histidine peptide, such as the tag provided in a pQE vector (QIAGEN, Inc., 9259 Eton Avenue, Chatsworth, Calif., 91311), among others, many of which are commercially available. As described in Gentz et al., 1989, Proc. Natl. Acad. Sci. USA 86:821-824, for instance, hexa-histidine provides for convenient purification of the fusion protein. Other peptide tags useful for purification include, but are not limited to, the hemagglutinin (“HA”) tag, which corresponds to an epitope derived from the influenza hemagglutinin protein (Wilson et al., 1984, Cell 37:767), and the “flag” tag.

In other embodiments, antibodies of the present invention or fragments thereof conjugated to a diagnostic or detectable agent. Such antibodies can be useful for monitoring or prognosing the onset, development, progression and/or severity of a disease or disorder (for example, but not limited to, an autoimmune disorder) as part of a clinical testing procedure, such as determining the efficacy of a particular therapy. Such diagnosis and detection can accomplished by coupling the antibody to detectable substances including, but not limited to, various enzymes, such as, but not limited to, horseradish peroxidase, alkaline phosphatase, beta-galactosidase, or acetylcholinesterase; prosthetic groups, such as, but not limited to, streptavidinlbiotin and avidin/biotin; fluorescent materials, such as, but not limited to, umbelliferone, fluorescein, fluorescein isothiocynate, rhodamine, dichlorotriazinylamine fluorescein, dansyl chloride or phycoerythrin; luminescent materials, such as, but not limited to, luminol; bioluminescent materials, such as but not limited to, luciferase, luciferin, and aequorin; radioactive materials, such as, but not limited to, iodine (¹³¹I, ¹²⁵I, ¹²³I, and ¹²¹I,), carbon (¹⁴C), sulfur (³⁵S), tritium (³H), indium (¹¹⁵In, 113In, ¹¹²In, and ¹¹¹In,), technetium (⁹⁹Tc), thallium (²⁰¹Ti), gallium (⁶⁸Ga, ⁶⁷Ga), palladium (¹⁰³Pd), molybdenum (⁹⁹Mo), xenon (¹³³Xe), fluorine (¹⁸F) ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, (¹⁸F), ¹⁵³Sm, ¹⁷⁷Lu, ¹⁵⁹Gd, ¹⁴⁹Pm, ¹⁴⁰La, ¹⁷⁵Yb, ¹⁶⁶Ho, ⁹⁰Y, ⁴⁷Sc, ¹⁸⁶Re, ¹⁸⁸Re, ¹⁴²Pr, ¹⁰⁵Rh, ⁹⁷Ru, ⁶⁸Ge, ₅₇Co, ₆₅Zn, ⁸⁵Sr, ³²P, ¹⁵³Gd, ₁₆₉Yb, ⁵¹Cr, ⁵⁴Mn, ⁷⁵Se, ¹¹³Sn, and ¹¹⁷Sn; and positron emitting metals using various positron emission tomographies, and noradioactive paramagnetic metal ions.

Alternatively, an antibody can be conjugated to a second antibody to form an antibody heteroconjugate as described by Segal in U.S. Pat. No. 4,676,980, which is incorporated herein by reference in its entirety.

The therapeutic moiety or drug conjugated to an antigen of interest (e.g., an interferon alpha polypeptide) or fragment thereof should be chosen to achieve the desired prophylactic or therapeutic effect(s) for a particular disease or disorder, for example, a disease or disorder associated with or characterized by aberrant expression and/or activity of an interferon alpha polypeptide, a disease or disorder associated with or characterized by aberrant expression and/or activity of the interferon alpha receptro or one or more subunits thereof, an autoimmune disease, an autoimmune disease, transplant rejection, graft versus host disease, or one or more symptoms thereof, in a subject. A clinician or other medical personnel should consider the following when deciding on what to conjugate to an antibody of interest, for example, an antibody that specifically binds to an interferon alpha polypeptide or fragment thereof: the nature of the disease, the severity of the disease, and the condition of the subject.

Antibodies may also be attached to solid supports, which are particularly useful for immunoassays or purification of the target antigen. Such solid supports include, but are not limited to, glass, cellulose, polyacrylamide, nylon, polystyrene, polyvinyl chloride or polypropylene.

### 5.4. Method of Preparing the Antibody Formulations

The present invention provides methods for preparing liquid formulations of antibodies or derivatives, analogues, or fragments thereof that specifically bind to an an antigen of interest (e.g., an interferon alpha polypeptide). The methods for preparing liquid formulations of the present invention may comprise: purifying the antibody (including antibody fragment thereof) from conditioned medium (either single lots or pooled lots of medium) and concentrating a fraction of the purified antibody (including antibody fragment thereof) to a final concentration of about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml. Conditioned medium containing the antibody (including antibody fragment thereof), for example, an antibody that specifically binds to an interferon alpha polypeptide may be subjected to CUNO filtration and the filtered antibody is subjected to HS50 cation exchange chromatography. The fraction from the HS50 cation exchange chromatography is then subjected to low pH treatment followed by MEP Hypercel chromatography. The fraction from the MEP Hypercel chromatography is subject to nanofiltration. The purified antibody or a fragment thereof obtained after nanofiltration is then subjected to diafiltration and ultrafiltration to buffer exchange and concentrate into the formulation buffer using the same membrane. For a detailed description for preparation of the antibody formulations, see Examples.

The liquid formulations of the present invention can be prepared as unit dosage forms by preparing a vial containing an aliquot of the liquid formulation for a one-time use. For example, a unit dosage per vial may contain 1 ml, 2 ml, 3 ml, 4 ml, 5 ml, 6 ml, 7 ml, 8 ml, 9 ml, 10 ml, 15 ml, or 20 ml of different concentrations of an antibody (including antibody fragment thereof) that specifically binds to an interferon alpha polypeptide ranging from about 10 mg/ml to about 300 mg/ml. If necessary, these preparations can be adjusted to a desired concentration by adding a sterile diluent to each vial. In a specific embodiment, the liquid formulations of the present invention are formulated into single dose vials as a sterile liquid that contains 25 mM histidine buffer at pH 6.0, 8% trehalose and 0.02% polysorbate 80. Each 1.0 mL of solution contains 100 mg of the antibody (including antibody fragment thereof). In one embodiment, the antibody (including antibody fragment thereof) of the invention is supplied at 100 mg/ml in 3 cc USP Type I borosilicate amber vials (West Pharmaceutical Services—Part No. 6800-0675). The target fill volume is 1.2 mL.

The liquid formulations of the present invention can be prepared as unit dosage forms by preparing a pre-filled syringe containing an aliquot of the liquid formulation for a one-time use. For example, a unit dosage per pre-filled syringe may contain 0.1 ml, 0.2 ml, 0.3 ml, 0.4 ml, 0.5 ml, 0.6 ml, 0.7 ml, 0.8 ml, 0.9 ml, 1 ml, 2 ml, 3 ml, 4 ml, 5 ml, 6 ml, 7 ml, 8 ml, 9 ml, 10 ml, 15 ml, or 20 ml of different concentrations of an antibody (including antibody fragment thereof) that specifically binds to an interferon alpha polypeptide ranging from about 10 mg/ml to about 300 mg/ml. In a specific embodiment, the liquid formulations of the present invention are formulated into single dose pre-filled syringes as a sterile liquid that contains 25 mM histidine buffer at pH 6.0, 8% trehalose and 0.02% polysorbate 80. Each 1.0 mL of solution contains 100 mg of the antibody (including antibody fragment thereof).

The liquid formulations of the present invention may be sterilized by various sterilization methods, including sterile filtration, radiation, etc. In a specific embodiment, the difiltrated antibody formulation is filter-sterilized with a presterilized 0.2 micron filter. Sterilized liquid formulations of the present invention may be administered to a subject to prevent, treat and/or manage a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof.

Although the invention is directed to liquid non-lyophilized formulations, it should be noted for the purpose of equivalents that the formulations of the invention may be lyophilized if desired. Thus, the invention encompasses lyophilized forms of the formulations of the invention.

### 5.5. Methods of Preparing Antibodies

The antibodies (including antibody fragments thereof) that specifically bind to an antigen can be produced by any method known in the art for the synthesis of antibodies, in particular, by chemical synthesis or by recombinant expression techniques (see, US Patent Publication 2007/0014724A1).

Polyclonal antibodies specific for an antigen can be produced by various procedures well-known in the art. For example, a human antigen can be administered to various host animals including, but not limited to, rabbits, mice, rats, etc. to induce the production of sera containing polyclonal antibodies specific for the human antigen. Various adjuvants may be used to increase the immunological response, depending on the host species, and include but are not limited to, Freund's (complete and incomplete), mineral gels such as aluminum hydroxide, surface active substances such as lysolecithin, pluronic polyols, polyanions, peptides, oil emulsions, keyhole limpet hemocyanins, dinitrophenol, and potentially useful human adjuvants such as BCG (bacille Calmette-Gucrin) and corynebacterium parvum. Such adjuvants are also well known in the art.

Monoclonal antibodies can be prepared using a wide variety of techniques known in the art including the use of hybridoma, recombinant, and phage display technologies, or a combination thereof. For example, monoclonal antibodies can be produced using hybridoma techniques including those known in the art and taught, for example, in Harlow et al., Antibodies: A Laboratory Manual, (Cold Spring Harbor Laboratory Press, 2nd ed. 1988); Hammerling, et al., in: Monoclonal Antibodies and T-Cell Hybridomas 563-681 (Elsevier, N.Y., 1981), and Harlow et al., Using Antibodies: A laboratory Manual, Cold Spring Harbor Laboratory Press (1999) (said references incorporated by reference in their entireties). The term “monoclonal antibody” as used herein is not limited to antibodies produced through hybridoma technology. The term “monoclonal antibody” refers to an antibody that is derived from a single clone, including any eukaryotic, prokaryotic, or phage clone, and not the method by which it is produced.

Methods for producing and screening for specific antibodies using hybridoma technology are routine and well known in the art. Briefly, mice can be immunized with a non-murine antigen and once an immune response is detected, e.g., antibodies specific for the antigen are detected in the mouse serum, the mouse spleen is harvested and splenocytes isolated. The splenocytes are then fused by well known techniques to any suitable myeloma cells, for example cells from cell line SP20 available from the ATCC. Hybridomas are selected and cloned by limited dilution. Additionally, a RIMMS (repetitive immunization multiple sites) technique can be used to immunize an animal (Kilpatrack et al., 1997, Hybridoma 16:381-9, incorporated herein by reference in its entirety). The hybridoma clones are then assayed by methods known in the art for cells that secrete antibodies capable of binding a polypeptide of the invention. Ascites fluid, which generally contains high levels of antibodies, can be generated by immunizing mice with positive hybridoma clones.

The present invention provides methods of generating monoclonal antibodies as well as antibodies produced by the method comprising culturing a hybridoma cell secreting an antibody of the invention wherein the hybridoma is generated by fusing splenocytes isolated from a mouse immunized with a non-murine antigen with myeloma cells and then screening the hybridomas resulting from the fusion for hybridoma clones that secrete an antibody able to bind to the antigen.

Antibody fragments which recognize specific particular epitopes may be generated by any technique known to those of skill in the art. For example, Fab and F(ab′)2 fragments of the invention may be produced by proteolytic cleavage of immunoglobulin molecules, using enzymes such as papain (to produce Fab fragments) or pepsin (to produce F(ab′)2 fragments). F(ab′)2 fragments contain the variable region, the light chain constant region and the CH1 domain of the heavy chain. Further, the antibodies of the present invention can also be generated using various phage display methods known in the art.

In phage display methods, functional antibody domains are displayed on the surface of phage particles which carry the polynucleotide sequences encoding them. In particular, DNA sequences encoding VH and VL domains are amplified from animal cDNA libraries (e.g., human or murine cDNA libraries of affected tissues). The DNA encoding the VH and VL domains are recombined together with an scFv linker by PCR and cloned into a phagemid vector. The vector is electroporated in E. coli and the E. coli is infected with helper phage. Phage used in these methods are typically filamentous phage including fd and M13 and the VH and VL domains are usually recombinantly fused to either the phage gene III or gene VIII. Phage expressing an antigen binding domain that binds to a particular antigen can be selected or identified with antigen, e.g., using labeled antigen or antigen bound or captured to a solid surface or bead. Examples of phage display methods that can be used to make the antibodies of the present invention include those disclosed in Brinkman et al., 1995, J. Immunol. Methods 182:41-50; Ames et al., 1995, J. Immunol. Methods 184:177-186; Kettleborough et al., 1994, Eur. J. Immunol. 24:952-958; Persic et al., 1997, Gene 187:9-18; Burton et al., 1994, Advances in Immunology 57:191-280; International application No. PCT/GB91/O1 134; International Publication Nos. WO 90/02809, WO 91/10737, WO 92/01047, WO 92/18619, WO 93/11236, WO 95/15982, WO 95/20401, and WO97/13844; and U.S. Pat. Nos. 5,698,426, 5,223,409, 5,403,484, 5,580,717, 5,427,908, 5,750,753, 5,821,047, 5,571,698, 5,427,908, 5,516,637, 5,780,225, 5,658,727, 5,733,743, 5,969,108, 6,33,187, 5,824,520, and 5,702,892; each of which is incorporated herein by reference in its entirety.

As described in the above references, after phage selection, the antibody coding regions from the phage can be isolated and used to generate whole antibodies, including human antibodies, or any other desired antigen binding fragment, and expressed in any desired host, including mammalian cells, insect cells, plant cells, yeast, and bacteria, e.g., as described below. Techniques to recombinantly produce Fab, Fab′ and F(ab′)2 fragments can also be employed using methods known in the art such as those disclosed in PCT publication No. WO 92/22324; Mullinax et al., 1992, BioTechniques 12(6):864-869; Sawai et al., 1995, AJRI 34:26-34; and Better et al., 1988, Science 240:1041-1043 (said references incorporated by reference in their entireties).

To generate whole antibodies, PCR primers including VH or VL nucleotide sequences, a restriction site, and a flanking sequence to protect the restriction site can be used to amplify the VH or VL sequences in scFv clones. Utilizing cloning techniques known to those of skill in the art, the PCR amplified VH domains can be cloned into vectors expressing a VH constant region, e.g., the human gamma 4 constant region, and the PCR amplified VL domains can be cloned into vectors expressing a VL constant region, e.g., human kappa or lamba constant regions. The vectors for expressing the VH or VL domains may comprise an EF-1α promoter, a secretion signal, a cloning site for the variable domain, constant domains, and a selection marker such as neomycin. The VH and VL domains may also cloned into one vector expressing the necessary constant regions. The heavy chain conversion vectors and light chain conversion vectors are then co-transfected into cell lines to generate stable or transient cell lines that express full-length antibodies, for example, but not limited to, IgG, using techniques known to those of skill in the art.

For some uses, including in vivo use of antibodies in humans and in vitro detection assays, it may be appropriate to use humanized antibodies or chimeric antibodies. Completely human antibodies and humanized antibodies are particularly desirable for therapeutic treatment of human subjects. Human antibodies can be made by a variety of methods known in the art including phage display methods described above using antibody libraries derived from human immunoglobulin sequences. See also U.S. Pat. Nos. 4,444,887 and 4,716,111; and International Publication Nos. WO 98/46645, WO 98/50433, WO 98/24893, WO98/16654, WO 96/34096, WO 96/33735, and WO 91/10741; each of which is incorporated herein by reference in its entirety.

Human antibodies can also be produced using transgenic mice which are incapable of expressing functional endogenous immunoglobulins, but which can express human immunoglobulin genes. For example, the human heavy and light chain immunoglobulin gene complexes may be introduced randomly or by homologous recombination into mouse embryonic stem cells. Alternatively, the human variable region, constant region, and diversity region may be introduced into mouse embryonic stem cells in addition to the human heavy and light chain genes. The mouse heavy and light chain immunoglobulin genes may be rendered non-functional separately or simultaneously with the introduction of human immunoglobulin loci by homologous recombination. In particular, homozygous deletion of the JH region prevents endogenous antibody production. The modified embryonic stem cells are expanded and microinjected into blastocysts to produce chimeric mice. The chimeric mice are then be bred to produce homozygous offspring which express human antibodies. The transgenic mice are immunized in the normal fashion with a selected antigen, e.g., all or a portion of a polypeptide of the invention. Monoclonal antibodies directed against the antigen can be obtained from the immunized, transgenic mice using conventional hybridoma technology. The human immunoglobulin transgenes harbored by the transgenic mice rearrange during B cell differentiation, and subsequently undergo class switching and somatic mutation. Thus, using such a technique, it is possible to produce therapeutically useful IgG, IgA, IgM and IgE antibodies. For an overview of this technology for producing human antibodies, see Lonberg and Huszar (1995, Int. Rev. Immunol. 13:65-93). For a detailed discussion of this technology for producing human antibodies and human monoclonal antibodies and protocols for producing such antibodies, see, e.g., International Publication Nos. WO 98/24893, WO 96/34096, and WO 96/33735; and U.S. Pat. Nos. 5,413,923, 5,625,126, 5,633,425, 5,569,825, 5,661,016, 5,545,806, 5,814,318, and 5,939,598, which are incorporated by reference herein in their entirety. In addition, companies such as Abgenix, Inc. (Freemont, Calif.) and Genpharm (San Jose, Calif.) can be engaged to provide human antibodies directed against a selected antigen using technology similar to that described above.

A chimeric antibody is a molecule in which different portions of the antibody are derived from different immunoglobulin molecules. Methods for producing chimeric antibodies are known in the art. See e.g., Morrison, 1985, Science 229:1202; Oi et al., 1986, BioTechniques 4:214; Gillies et al., 1989, J. Immunol. Methods 125:191-202; and U.S. Pat. Nos. 5,807,715, 4,816,567, 4,816,397, and 6,331,415, which are incorporated herein by reference in their entirety.

A humanized antibody is an antibody or its variant or fragment thereof which is capable of binding to a predetermined antigen and which comprises a framework region having substantially the amino acid sequence of a human immunoglobulin and a CDR having substantially the amino acid sequence of a non-human immuoglobulin. A humanized antibody comprises substantially all of at least one, and typically two, variable domains (Fab, Fab′, F(ab′)₂, Fabc, Fv) in which all or substantially all of the CDR regions correspond to those of a non-human immunoglobulin (i.e., donor antibody) and all or substantially all of the framework regions are those of a human immunoglobulin consensus sequence. In one embodiment, a humanized antibody also comprises at least a portion of an immunoglobulin constant region (Fc), typically that of a human immunoglobulin. Ordinarily, the antibody will contain both the light chain as well as at least the variable domain of a heavy chain. The antibody also may include the CH1, hinge, CH2, CH3, and CH4 regions of the heavy chain. The humanized antibody can be selected from any class of immunoglobulins, including IgM, IgG, IgD, IgA and IgE, and any isotype, including IgG₁, IgG₂, IgG₃ and IgG₄. Usually the constant domain is a complement fixing constant domain where it is desired that the humanized antibody exhibit cytotoxic activity, and the class is typically IgG₁. Where such cytotoxic activity is not desirable, the constant domain may be of the IgG₂ class. The humanized antibody may comprise sequences from more than one class or isotype, and selecting particular constant domains to optimize desired effector functions is within the ordinary skill in the art. The framework and CDR regions of a humanized antibody need not correspond precisely to the parental sequences, e.g., the donor CDR or the consensus framework may be mutagenized by substitution, insertion or deletion of at least one residue so that the CDR or framework residue at that site does not correspond to either the consensus or the import antibody. Such mutations, however, will not be extensive. Usually, at least 75% of the humanized antibody residues will correspond to those of the parental framework and CDR sequences, more often 90%, and greater than 95%. Humanized antibody can be produced using variety of techniques known in the art, including but not limited to, CDR-grafting (European Patent No. EP 239,400; International publication No. WO 91/09967; and U.S. Pat. Nos. 5,225,539, 5,530,101, and 5,585,089), veneering or resurfacing (European Patent Nos. EP 592,106 and EP 519,596; Padlan, 1991, Molecular Immunology 28(4/5):489-498; Studnicka et al., 1994, Protein Engineering 7(6):805-814; and Roguska et al., 1994, PNAS 91:969-973), chain shuffling (U.S. Pat. No. 5,565,332), and techniques disclosed in, e.g., U.S. Pat. No. 6,407,213, U.S. Pat. No. 5,766,886, WO 9317105, Tan et al., J. Immunol. 169:1119-25 (2002), Caldas et al., Protein Eng. 13(5):353-60 (2000), Morca et al., Methods 20(3):267-79 (2000), Baca et al., J. Biol. Chem. 272(16):10678-84 (1997), Roguska et al., Protein Eng. 9(10):895-904 (1996), Couto et al., Cancer Res. 55 (23 Supp):5973s-5977s (1995), Couto et al., Cancer Res. 55(8):1717-22 (1995), Sandhu J S, Gene 150(2):409-10 (1994), and Pedersen et al., J. Mol. Biol. 235(3):959-73 (1994). Often, framework residues in the framework regions will be substituted with the corresponding residue from the CDR donor antibody to alter, preferably improve, antigen binding. These framework substitutions are identified by methods well known in the art, for example, but not limited to, by modeling of the interactions of the CDR and framework residues to identify framework residues important for antigen binding and sequence comparison to identify unusual framework residues at particular positions (see, e.g., Queen et al., U.S. Pat. No. 5,585,089; and Riechmann et al., 1988, Nature 332:323, which are incorporated herein by reference in their entireties).

Single domain antibodies, for example, antibodies lacking the light chains, can be produced by methods well-known in the art. See Riechmann et al., 1999, J. Immuno. 231:25-38; Nuttall et al., 2000, Curr. Pharm. Biotechnol. 1(3):253-263; Muylderman, 2001, J. Biotechnol. 74(4):277302; U.S. Pat. No. 6,005,079; and International Publication Nos. WO 94/04678, WO 94/25591, and WO 01/44301, each of which is incorporated herein by reference in its entirety.

Further, the antibodies that specifically bind to an antigen (e.g., an interferon alpha polypeptide) can, in turn, be utilized to generate anti-idiotype antibodies that “mimic” an antigen using techniques well known to those skilled in the art. (See, e.g., Greenspan & Bona, 1989, FASEB J. 7(5):437-444; and Nissinoff, 1991, J. Immunol. 147(8):2429-2438).

### 5.5.1. Recombinant Expression of an Antibody

Recombinant expression of an antibody contained in a formulation of the invention (e.g., a heavy or light chain of an antibody of the invention or a fragment thereof or a single chain antibody of the invention) may require construction of an expression vector containing a polynucleotide that encodes the antibody. Once a polynucleotide encoding an antibody molecule, heavy or light chain of an antibody, or fragment thereof has been obtained, the vector for the production of the antibody molecule may be produced by recombinant DNA technology using techniques well-known in the art. Thus, methods for preparing a protein by expressing a polynucleotide containing an antibody encoding nucleotide sequence are described herein. Methods which are well known to those skilled in the art can be used to construct expression vectors containing antibody coding sequences and appropriate transcriptional and translational control signals. These methods include, for example, in vitro recombinant DNA techniques, synthetic techniques, and in vivo genetic recombination. The invention, thus, provides replicable vectors comprising a nucleotide sequence encoding an antibody molecule of the invention, a heavy or light chain of an antibody, a heavy or light chain variable domain of an antibody (including antibody fragment thereof), or a heavy or light chain CDR, operably linked to a promoter. Such vectors may include the nucleotide sequence encoding the constant region of the antibody molecule (see, e.g., International Publication No. WO 86/05807; International Publication No. WO 89/01036; and U.S. Pat. No. 5,122,464) and the variable domain of the antibody may be cloned into such a vector for expression of the entire heavy, the entire light chain, or both the entire heavy and light chains.

The expression vector is transferred to a host cell by conventional techniques and the transfected cells are then cultured by conventional techniques to produce an antibody of the invention. Thus, the invention includes host cells containing a polynucleotide encoding an antibody of the invention or fragments thereof, or a heavy or light chain thereof, or fragment thereof, or a single chain antibody of the invention, operably linked to a heterologous promoter. In specific embodiments for the expression of double-chained antibodies, vectors encoding both the heavy and light chains may be co-expressed in the host cell for expression of the entire immunoglobulin molecule, as detailed below.

A variety of host-expression vector systems may be utilized to express the antibody molecules of the invention (see, e.g., U.S. Pat. No. 5,807,715). Such host-expression systems represent vehicles by which the coding sequences of interest may be produced and subsequently purified, but also represent cells which may, when transformed or transfected with the appropriate nucleotide coding sequences, express an antibody molecule of the invention in situ. These include but are not limited to microorganisms such as bacteria (for example, but not limited to, E. coli and B. subtilis) transformed with recombinant bacteriophage DNA, plasmid DNA or cosmid DNA expression vectors containing antibody coding sequences; yeast (for example, but not limited to, Saccharomyces Pichia) transformed with recombinant yeast expression vectors containing antibody coding sequences; insect cell systems infected with recombinant virus expression vectors (for example, but not limited to, baculovirus) containing antibody coding sequences; plant cell systems infected with recombinant virus expression vectors (for example, but not limited to, cauliflower mosaic virus, CaMV; tobacco mosaic virus, TMV) or transformed with recombinant plasmid expression vectors (for example, but not limited to, Ti plasmid) containing antibody coding sequences; or mammalian cell systems (for example, but not limited to, COS, CHO, BHK, 293, NSO, and 3T3 cells) harboring recombinant expression constructs containing promoters derived from the genome of mammalian cells (for example, but not limited to, metallothionein promoter) or from mammalian viruses (for example, but not limited to, the adenovirus late promoter; the vaccinia virus 7.5K promoter). Bacterial cells such as Escherichia coli, and cukaryotic cells, especially for the expression of whole recombinant antibody molecule, are used for the expression of a recombinant antibody molecule. For example, mammalian cells such as Chinese hamster ovary cells (CHO), in conjunction with a vector such as the major intermediate early gene promoter element from human cytomegalovirus is an effective expression system for antibodies (Foecking et al., 1986, Gene 45:101; and Cockett et al., 1990, Bio/Technology 8:2). In a specific embodiment, the expression of nucleotide sequences encoding antibodies of the invention, derivative, analog, or fragment thereof is regulated by a constitutive promoter, inducible promoter or tissue specific promoter.

In bacterial systems, a number of expression vectors may be advantageously selected depending upon the use intended for the antibody molecule being expressed. For example, when a large quantity of such an antibody is to be produced, for the generation of pharmaceutical compositions of an antibody molecule, vectors which direct the expression of high levels of fusion protein products that are readily purified may be desirable. Such vectors include, but are not limited to, the E. coli expression vector pUR278 (Ruther et al., 1983, EMBO 12:1791), in which the antibody coding sequence may be ligated individually into the vector in frame with the lac Z coding region so that a fusion protein is produced; pIN vectors (Inouye & Inouye, 1985, Nucleic Acids Res. 13:3101-3109; Van Heeke & Schuster, 1989, J. Biol. Chem. 24:5503-5509); and the like. pGEX vectors may also be used to express foreign polypeptides as fusion proteins with glutathione 5-transferase (GST). In general, such fusion proteins are soluble and can easily be purified from lysed cells by adsorption and binding to matrix glutathione agarose beads followed by elution in the presence of free glutathione. The pGEX vectors are designed to include thrombin or factor Xa protease cleavage sites so that the cloned target gene product can be released from the GST moiety.

In an insect system, Autographa californica nuclear polyhedrosis virus (AcNPV) is used as a vector to express foreign genes. The virus grows in Spodoptera frugiperda cells. The antibody coding sequence may be cloned individually into non-essential regions (for example the polyhedrin gene) of the virus and placed under control of an AcNPV promoter (for example the polyhedrin promoter).

In mammalian host cells, a number of viral-based expression systems may be utilized. In cases where an adenovirus is used as an expression vector, the antibody coding sequence of interest may be ligated to an adenovirus transcription/translation control complex, e.g., the late promoter and tripartite leader sequence. This chimeric gene may then be inserted in the adenovirus genome by in vitro or in vivo recombination. Insertion in a non-essential region of the viral genome (e.g., region E1 or E3) will result in a recombinant virus that is viable and capable of expressing the antibody molecule in infected hosts (e.g., see Logan & Shenk, 1984, Proc. Natl. Acad. Sci. USA 8 1:355-359). Specific initiation signals may also be required for efficient translation of inserted antibody coding sequences. These signals include the ATG initiation codon and adjacent sequences. Furthermore, the initiation codon must be in phase with the reading frame of the desired coding sequence to ensure translation of the entire insert. These exogenous translational control signals and initiation codons can be of a variety of origins, both natural and synthetic. The efficiency of expression may be enhanced by the inclusion of appropriate transcription enhancer elements, transcription terminators, etc. (see, e.g., Bittner et al., 1987, Methods in Enzymol. 153:51-544).

In addition, a host cell strain may be chosen which modulates the expression of the inserted sequences, or modifies and processes the gene product in the specific fashion desired. Such modifications (for example, but not limited to, glycosylation) and processing (for example, but not limited to, cleavage) of protein products may be important for the function of the protein. Different host cells have characteristic and specific mechanisms for the post-translational processing and modification of proteins and gene products. Appropriate cell lines or host systems can be chosen to ensure the correct modification and processing of the foreign protein expressed. To this end, eukaryotic host cells which possess the cellular machinery for proper processing of the primary transcript, glycosylation, and phosphorylation of the gene product may be used. Such mammalian host cells include but are not limited to CHO, VERY, BHK, Hela, COS, MDCK, 293, 3T3, W138, BT483, Hs578T, HTB2, BT20 and T47D, NSO (a murine myeloma cell line that does not endogenously produce any immunoglobulin chains), CRL7030 and HsS78Bst cells.

For long-term, high-yield production of recombinant proteins, stable expression is may be used. For example, cell lines which stably express the antibody molecule may be engineered. Rather than using expression vectors which contain viral origins of replication, host cells can be transformed with DNA controlled by appropriate expression control elements (e.g., promoter, enhancer, sequences, transcription terminators, polyadenylation sites, etc.), and a selectable marker. Following the introduction of the foreign DNA, engineered cells may be allowed to grow for 1-2 days in an enriched media, and then are switched to a selective media. The selectable marker in the recombinant plasmid confers resistance to the selection and allows cells to stably integrate the plasmid into their chromosomes and grow to form foci which in turn can be cloned and expanded into cell lines. This method may advantageously be used to engineer cell lines which express the antibody molecule. Such engineered cell lines may be particularly useful in screening and evaluation of compositions that interact directly or indirectly with the antibody molecule.

A number of selection systems may be used, including but not limited to, the herpes simplex virus thymidine kinase (Wigler et al., 1977, Cell 11:223), hypoxanthineguanine phosphoribosyltransferase (Szybalska & Szybalski, 1992, Proc. Natl. Acad. Sci. USA 48:202), and adenine phosphoribosyltransferase (Lowy et al., 1980, Cell 22:8-17) genes can be employed in tk-, hgprt- or aprt-cells, respectively. Also, antimetabolite resistance can be used as the basis of selection for the following genes: dhfr, which confers resistance to methotrexate (Wigler et al., 1980, Natl. Acad. Sci. USA 77:357; O'Hare et al., 1981, Proc. Natl. Acad. Sci. USA 78:1527); gpt, which confers resistance to mycophenolic acid (Mulligan & Berg, 1981, Proc. Natl. Acad. Sci. USA 78:2072); neo, which confers resistance to the aminoglycoside G-418 (Wu and Wu, 1991, Biotherapy 3:87-95; Tolstoshev, 1993, Ann. Rev. Pharmacol. Toxicol. 32:573-596; Mulligan, 1993, Science 260:926-932; and Morgan and Anderson, 1993, Ann. Rev. Biochem. 62: 191-217; May, 1993, TIB TECH 11(5):155-215); and hygro, which confers resistance to hygromycin (Santerre et al., 1984, Gene 30:147). Methods commonly known in the art of recombinant DNA technology may be routinely applied to select the desired recombinant clone, and such methods are described, for example, in Ausubel et al. (eds.), Current Protocols in Molecular Biology, John Wiley & Sons, NY (1993); Kriegler, Gene Transfer and Expression, A Laboratory Manual, Stockton Press, NY (1990); and in Chapters 12 and 13, Dracopoli et al. (eds), Current Protocols in Human Genetics, John Wiley & Sons, NY (1994); Colberre-Garapin et al., 1981, J. Mol. Biol. 150:1, which are incorporated by reference herein in their entireties.

The expression levels of an antibody molecule can be increased by vector amplification (for a review, see Bebbington and Hentschel, The use of vectors based on gene amplification for the expression of cloned genes in mammalian cells in DNA cloning, Vol. 3. (Academic Press, New York, 1987)). When a marker in the vector system expressing antibody is amplifiable, increase in the level of inhibitor present in culture of host cell will increase the number of copies of the marker gene. Since the amplified region is associated with the antibody gene, production of the antibody will also increase (Crouse et al., 1983, Mol. Cell. Biol. 3:257).

The host cell may be co-transfected with two expression vectors of the invention, the first vector encoding a heavy chain derived polypeptide and the second vector encoding a light chain derived polypeptide. The two vectors may contain identical selectable markers which enable equal expression of heavy and light chain polypeptides. Alternatively, a single vector may be used which encodes, and is capable of expressing, both heavy and light chain polypeptides. In such situations, the light chain should be placed before the heavy chain to avoid an excess of toxic free heavy chain (Proudfoot, 1986, Nature 322:52; and Kohler, 1980, Proc. Natl. Acad. Sci. USA 77:2 197). The coding sequences for the heavy and light chains may comprise cDNA or genomic DNA.

Once an antibody molecule of the invention has been produced by recombinant expression, it may be purified by any method known in the art for purification of an immunoglobulin molecule, for example, by chromatography (e.g., ion exchange, affinity, particularly by affinity for the specific antigen after Protein A, and sizing column chromatography), centrifugation, differential solubility, or by any other standard technique for the purification of proteins. Further, the antibodies of the present invention or fragments thereof may be fused to heterologous polypeptide sequences described herein or otherwise known in the art to facilitate purification.

### 5.6. Methods of Monitoring the Stability and Aggregation of Antibody Formulations

There are various methods available for assessing the stability of protein formulations, including antibody formulations, based on the physical and chemical structures of the proteins as well as on their biological activities. For example, to study denaturation of proteins, methods such as charge-transfer absorption, thermal analysis, fluorescence spectroscopy, circular dichroism (CD), NMR, reducing capillary gel electrophoresis (rCGE) and high performance size exclusion chromatography (HPSEC), tangential flow filtration (TFF), static light scattering (SLS), Fourier Transform Infrared Spectroscopy (FTIR), urea-induced protein unfolding techniques, intrinsic tryptophan fluorescence, differential scanning calorimetry, and 1-anilino-8-naphthalenesulfonic acid (ANS) protein binding techniques are available. See, for example, Wang et al., 1988, J. of Parenteral Science & Technology 42(Suppl):S4-S26.

rCGE and HPSEC are the most common and simplest methods to assess the formation of protein aggregates, protein degradation, and protein fragmentation. Accordingly, the stability of the liquid formulations of the present invention may be assessed by these methods.

For example, the stability of the liquid formulations of the present invention may be evaluated by HPSEC, wherein the percent area of the peaks represents the non-degraded antibody or non-degraded antibody fragments. In particular, approximately 250 μg of the antibody (including antibody fragment thereof) (approximately 25 μl of a liquid formulation comprising 10 mg/ml said antibody or antibody fragment) is injected onto a TosoH Biosep TSK G3000SW_{XL} column (7.8 mm×30 cm) fitted with a TSK SW×1 guard column (6.0 mm CX 4.0 cm). The antibody (including antibody fragment thereof) is eluted isocratically with 0.1 M disodium phosphate containing 0.1 M sodium sulfate and 0.05% sodium azide, at a flow rate of 0.8 to 1.0 ml/min. Eluted protein is detected using UV absorbance at 280 nm. Reference standards are run in the assay as controls, and the results are reported as the area percent of the product monomer peak compared to all other peaks excluding the included volume peak observed at approximately 12 to 14 minutes. Peaks eluting earlier than the monomer peak are recorded as percent aggregate.

The liquid formulations of the present invention exhibit low to undetectable levels of aggregation as measured by any of the methods described above, that is, no more than 5%, no more than 4%, no more than 3%, no more than 2%, no more than 1%, and no more than 0.5% aggregate by weight protein, and low to undetectable levels of fragmentation, that is, 80% or higher, 85% or higher, 90% or higher, 95% or higher, 98% or higher, or 99% or higher, or 99.5% or higher of the total peak area in the peak(s) representing intact antibodies (including antibody fragments thereof). When SDS-PAGE is used to measure antibody fragmentation, the density or the radioactivity of each band stained or labeled with radioisotope can be measured and the % density or % radioactivity of the band representing non-degraded antibodies (including antibody fragments thereof) can be obtained.

The stability of the liquid formulations of the present invention can be also assessed by any assays which measure the biological activity of the antibody in the formulation. The biological activities of antibodies include, but are not limited to, antigen-binding activity, blocking of ligand-receptor interaction, and so forth (see infra). Antigen-binding activity of the antibodies (including antibody fragments thereof) can be measured by any method known to those skilled in the art, including but not limited to ELISA, radioimmunoassay, Western blot, and the like. Also see Harlow et al., Antibodies: A Laboratory Manual, (Cold Spring Harbor Laboratory Press, 2nd ed. 1988) (incorporated by reference herein in its entirety). An ELISA based assay, e.g., may be used to compare the ability of an antibody (including antibody fragments thereof) to specifically bind to an interferon alpha polypeptide to that of a reference standards antibody.

The purity of the liquid antibody formulations of the invention may be measured by any method well-known to one of skill in the art such as, for example, but not limited to, HPSEC. The sterility of the liquid antibody formulations may be assessed by any method well-known to one of skill in the art such as, e.g: sterile soybean-casein digest medium and fluid thioglycollate medium are inoculated with a test liquid antibody formulation by filtering the liquid antibody formulation through a sterile filter having a nominal porosity of 0.45 μm. When using the Sterisure™ or Steritest™ method, each filter device is aseptically filled with approximately 100 ml of sterile soybean-casein digest medium or fluid thioglycollate medium. When using the conventional method, the challenged filter is aseptically transferred to 100 ml of sterile soybean-casein digest medium or fluid thioglycollate medium. The media are incubated at appropriate temperatures and observed three times over a 14 day period for evidence of bacterial or fungal growth.

### 5.7. Prophylactic and Therapeutic Utility of the Antibody Formulations

The present invention is also directed to antibody-based therapies which involve administering to a human subject the liquid antibody formulations (or “antibody formulations” or “liquid formulations”) of the present invention for preventing, treating and/or managing a disease or disorder, for example, a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, transplant rejection and graft versus host disease, or one or more symptoms thereof.

The antibody compositions of the invention can be used in the treatment of autoimmune diseases, such as systemic lupus erythematosus (SLE), multiple sclerosis (MS), inflammatory bowel disease (IBD; including Crohn's Disease, Ulcerative Colitis and Celiac's Disease), insulin dependent diabetes mellitus (IDDM), psoriasis, autoimmune thyroiditis, rheumatoid arthritis (RA) and glomerulonephritis. Furthermore, the antibody compositions of the invention can be used for inhibiting or preventing transplant rejection or in the treatment of graft versus host disease (GVHD).

The liquid formulations of the present invention may be used locally or systemically in the body as a therapeutic. Particularly, the liquid formulations of the invention may be used in the prevention, treatment and/or management of a disease or disorder, for example, a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof. The formulations of the invention can be used to regulate the activity of cells expressing an interferon alpha receptor. In a specific embodiment, the formulations of the invention are used to regulate various activities of a body, including but not limited to, immune functions. The formulations of the present invention may also be utilized in combination with one or more other therapies (e.g., one or more other prophylactic or therapeutic agents), for example, therapies useful in the prevention, treatment and/or management of a disease or disorder, for example, a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof. When one or more other therapies (e.g., prophylactic or therapeutic agents) are used, they can be administered separately, in any appropriate form and by any suitable route. Therapeutic or prophylactic agents include, but are not limited to, small molecules, synthetic drugs, peptides, polypeptides, proteins, nucleic acids (for example, but not limited to, DNA and RNA nucleotides including, but not limited to, antisense nucleotide sequences, triple helices, RNAi, and nucleotide sequences encoding biologically active proteins, polypeptides or peptides) antibodies, synthetic or natural inorganic molecules, mimetic agents, and synthetic or natural organic molecules.

Any therapy (e.g., prophylactic or therapeutic agents) which is known to be useful, or which has been used or is currently being used for the prevention, treatment and/or management of one or more symptoms associated with a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease can be used in combination with the liquid antibody formulations of the present invention in accordance with the invention described herein. See, e.g., Gilman et al., Goodman and Gilman's: The Pharmacological Basis of Therapeutics, Tenth Ed., McGraw-Hill, New York, 2001; The Merck Manual of Diagnosis and Therapy, Berkow, M. D. et al. (eds.), 17th Ed., Merck Sharp & Dohme Research Laboratories, Rahway, N.J., 1999; and Cecil Textbook of Medicine, 20th Ed., Bennett and Plum (eds.), W.B. Saunders, Philadelphia, 1996 for information regarding therapies, in particular prophylactic or therapeutic agents, which have been or are currently being used for preventing, treating and/or managing diseases or disorders associated with or characterized by aberrant expression and/or activity of an interferon alpha polypeptide, diseases or disorders associated with or characterized by aberrant expression and/or activity of the interferon alpha receptor or one or more subunits thereof, autoimmune diseases, inflammatory diseases, or one or more symptoms thereof. Examples of prophylactic and therapeutic agents include, but are not limited to, immunomodulatory agents, anti-inflammatory agents (for example, but not limited to, adrenocorticoids, corticosteroids (for example, but not limited to, beclomethasone, budesonide, flunisolide, fluticasone, triamcinolone, methlyprednisolone, prednisolone, prednisone, hydrocortisone), glucocorticoids, steroids, non-steriodal anti-inflammatory drugs (for example, but not limited to, aspirin, ibuprofen, diclofenac, and COX-2 inhibitors), and leukotreine antagonists (for example, but not limited to, montelukast, methyl xanthines, zafirlukast, and zileuton), beta2-agonists (for example, but not limited to, albuterol, biterol, fenoterol, isoetharie, metaproterenol, pirbuterol, salbutamol, terbutalin formoterol, salmeterol, and salbutamol terbutalinc), anticholinergic agents (for example, but not limited to, ipratropium bromide and oxitropium bromide), sulphasalazine, penicillamine, dapsone, antihistamines, anti-malarial agents (for example, but not limited to, hydroxychloroquine), anti-viral agents, and antibiotics (for example, but not limited to, dactinomycin (formerly actinomycin), bleomycin, erythomycin, penicillin, mithramycin, and anthramycin (AMC)).

A liquid formulation of the invention may be administered to a human concurrently with one or more other therapies (e.g., one or more other prophylactic or therapeutic agents) useful for the prevention, treatment and/or management of a disease or disorder associated with or characterized by aberrant expression and/or activity of an interferon alpha polypeptide, a disease or disorder associated with or characterized by aberrant expression and/or activity of the interferon alpha receptro or one or more subunits thereof, an autoimmune disease, an autoimmune disease, transplant rejection, graft versus host disease, or one or more symptoms thereof. The term “concurrently” is not limited to the administration of prophylactic or therapeutic agents/therapies at exactly the same time, but rather it is meant that a liquid formulation of the invention and the other agent/therapy are administered to a mammal in a sequence and within a time interval such that the antibody (including antibody fragment thereof) that specifically binds to an interferon alpha polypeptide contained in the liquid formulation can act together with the other agent/therapy to provide an increased benefit than if they were administered otherwise.

In various embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered less than 1 hour apart, at about 1 hour apart, at about 1 hour to about 2 hours apart, at about 2 hours to about 3 hours apart, at about 3 hours to about 4 hours apart, at about 4 hours to about 5 hours apart, at about 5 hours to about 6 hours apart, at about 6 hours to about 7 hours apart, at about 7 hours to about 8 hours apart, at about 8 hours to about 9 hours apart, at about 9 hours to about 10 hours apart, at about 10 hours to about 11 hours apart, at about 11 hours to about 12 hours apart, no more than 24 hours apart or no more than 48 hours apart. In specific embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered within the same patient visit. In other embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered at about 2 to 4 days apart, at about 4 to 6 days apart, at about 1 week part, at about 1 to 2 weeks apart, or more than 2 weeks apart. In specific embodiments, a liquid formulation of the invention and one or more other therapies (e.g., prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered in a time frame where both agents are still active. One skilled in the art would be able to determine such a time frame by determining the half-life of the administered agents.

In certain embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are cyclically administered to a subject. Cycling therapy involves the administration of a first agent for a period of time, followed by the administration of a second agent and/or third agent for a period of time and repeating this sequential administration. Cycling therapy can reduce the development of resistance to one or more of the therapies, avoid or reduce the side effects of one of the therapies, and/or improves the efficacy of the treatment.

In certain embodiments, a liquid formulation of the invention and one or more other therapies (e.g., one or more other prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered in a cycle of less than about 3 weeks, about once every two weeks, about once every 10 days or about once every week. One cycle can comprise the administration of a therapeutic or prophylactic agent by infusion over about 90 minutes every cycle, about 1 hour every cycle, about 45 minutes every cycle. Each cycle can comprise at least 1 week of rest, at least 2 weeks of rest, at least 3 weeks of rest. The number of cycles administered is from about 1 to about 12 cycles, more typically from about 2 to about 10 cycles, and more typically from about 2 to about 8 cycles.

In other embodiments, liquid formulation of the invention and one or more other therapies (e.g., prophylactic or therapeutic agents), preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, are administered in metronomic dosing regimens, either by continuous infusion or frequent administration without extended rest periods. Such metronomic administration can involve dosing at constant intervals without rest periods. Typically the prophylactic or therapeutic agents, in particular cytotoxic agents, are used at lower doses. Such dosing regimens encompass the chronic daily administration of relatively low doses for extended periods of time. In specific embodiments, the use of lower doses can minimize toxic side effects and eliminate rest periods. In certain embodiments, the prophylactic and therapeutic agents are delivered by chronic low-dose or continuous infusion ranging from about 24 hours to about 2 days, to about 1 week, to about 2 weeks, to about 3 weeks to about 1 month to about 2 months, to about 3 months, to about 4 months, to about 5 months, to about 6 months.

In one embodiment, a liquid formulation of the invention is administered in a dosing regimen that maintains the plasma concentration of the antibody (including antibody fragment thereof) specific for an interferon alpha polypeptide at a desirable level (e.g., about 0.1 to about 100 μg/ml), which continuously blocks the an interferon alpha receptor activity. In a specific embodiment, the plasma concentration of the antibody (including antibody fragment thereof) is maintained at 0.2 μg/ml, 0.5 μg/ml, 1 μg/ml, 2 μg/ml, 3 μg/ml, 4 μg/ml, 5 μg/ml, 6 μg/ml, 7 μg/ml, 8 μg/ml, 9 μg/ml, 10 μg/ml, 15 μg/ml, 20 μg/ml, 25 μg/ml, 30 μg/ml, 35 μg/ml, 40 μg/ml, 45 μg/ml or 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment and/or management of a chronic disease or disorder.

In one embodiment, a liquid formulation of the invention is administered to a subject with a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof using a dosing regimen that maintains the plasma concentration of the an antibody (including antibody fragment thereof) that specifically binds to an interferon alpha polypeptide at a level that blocks at least 40%, at least 50%, at least 55%, at least 60%, at least 65%, at least 70%, at least 75%, at least 80%, at least 85%, at least 90% or at least 95% of interferon alpha receptor binding to an interferon alpha polypeptide. In a specific embodiment, the plasma concentration of the an antibody (including antibody fragment thereof) that specifically binds to an interferon alpha polypeptide is maintained at about 0.1 μg/ml to about 100 μg/ml in a subject with a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof.

In some embodiments, a liquid formulation of the invention is administered intermittently to a subject, wherein the liquid formulation comprises an antibody (including antibody fragment thereof) conjugated to a moiety.

When used in combination with other therapies (e.g., prophylactic and/or therapeutic agents) useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, the liquid formulations of the invention and the other therapy can act additively or syncrgistically. The invention contemplates administration of a liquid formulation of the invention in combination with other therapies (e.g., prophylactic or therapeutic agents) preferably therapies useful for prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof by the same or different routes of administration, for example, but not limited to, oral and parenteral. In certain embodiments, when a liquid formulation of the invention is administered concurrently with one or more therapies (e.g., prophylactic or therapeutic agents) that potentially produce adverse side effects (including, but not limited to, toxicity), the therapies (e.g., prophylactic or therapeutic agents) can advantageously be administered at a dose that falls below the threshold that the adverse side effect is elicited.

### 5.7.1. Inflammatory Disorder Treatment

The liquid formulations of the invention may be administered to a subject in need thereof to prevent, treat and/or manage an inflammatory disorder (e.g., inflammatory bowel disease) or one or more symptoms thereof. The liquid formulations of the invention may also be administered in combination with one or more other therapies, preferably therapies useful for the prevention, treatment and/or management of an inflammatory disorder to a subject in need thereof to prevent, treat and/or manage an inflammatory disorder or one or more symptoms thereof. In a specific embodiment, the invention provides a method of preventing, treating and/or managing an inflammatory disorder or one or more symptoms thereof, said method comprising administering to a subject in need thereof a dose of a prophylactically or therapeutically effective amount of a liquid formulation of the invention. In another embodiment, the invention provides a method of preventing, treating and/or managing an inflammatory disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that specifically bind to an interferon alpha polypeptide.

The invention provides methods for preventing, treating and/or managing one or more symptoms of an inflammatory disorder in a subject refractory to conventional therapies (for example, but not limited to, methotrexate and a TNF-α antagonist (e.g., REMICADE™ or ENBREL™)) for such an inflammatory disorder, said methods comprising administering to said subject a dose of a prophylactically or therapeutically effective amount of a liquid formulation of the invention. The invention also provides methods for preventing, treating and/or managing one or more symptoms of an inflammatory disorder in a subject refractory to existing single agent therapies for such an inflammatory disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that specifically bind to an interferon alpha polypeptide. The invention also provides methods for managing or treating an inflammatory disorder by administering a liquid formulation of the invention in combination with any other treatment to patients who have proven refractory to other treatments but are no longer on these treatments. The invention also provides alternative methods for the treatment of an inflammatory disorder where another therapy has proven or may prove too toxic, i.e., results in unacceptable or unbearable side effects, for the subject being treated. For example, the liquid formulations of the invention may be administered to a subject, wherein the subject is refractory to a TNF antagonist or methotrexate. Further, the invention provides methods for preventing the recurrence of an inflammatory disorder in patients that have been treated and have no disease activity by administering a liquid formulation of the invention.

Inflammatory disorders that can be treated by the methods encompassed by the invention include, but are not limited to inflammatory bowel disease and psoriatic arthritis. As described herein, some autoimmune disorders are associated with an inflammatory condition.

Anti-inflammatory therapies and their dosages, routes of administration and recommended usage are known in the art and have been described in such literature as the Physicians' Desk Reference (60th ed., 2006).

### 5.7.1.1. Anti-Inflammatory Therapies

The present invention provides methods of preventing, treating and/or managing an inflammatory disorder or one or more symptoms thereof, said methods comprising administering to a subject in need thereof a liquid formulation of the invention and one or more therapies (e.g., prophylactic or therapeutic agents other than antibodies (including antibody fragments thereof) that specifically bind to an interferon alpha polypeptide. Any agent or therapy which is known to be useful, or which has been used or is currently being used for the prevention, treatment and/or management of an inflammatory disorder or one or more symptoms thereof can be used in combination with a liquid formulation of the invention in accordance with the invention described herein.

Any anti-inflammatory agent, including agents useful in therapies for inflammatory disorders, well-known to one of skill in the art can be used in the compositions and methods of the invention. Non-limiting examples of anti-inflammatory agents include non-steroidal anti-inflammatory drugs (NSAIDs), steroidal anti-inflammatory drugs, anticholinergics (for example, but not limited to, atropine sulfate, atropine methylnitrate, and ipratropium bromide (ATROVENT™)), beta2-agonists (for example, but not limited to, abuterol (VENTOLIN™ and PROVENTIL™), bitolterol (TORNALATE™), levalbuterol (XOPONEX™), metaproterenol (ALUPENT™), pirbuterol (MAXAIR™), terbutlaine (BRETHAIRE™ and BRETHINE™), albuterol (PROVENTIL™, REPETABS™, and VOLMAX™), formoterol (FORADIL AEROLIZER™), and salmeterol (SEREVENT™ and SEREVENT DISKUS™)), and methylxanthines (for example, but not limited to, theophylline (UNIPHYL™, THEO-DUR™, SLO-BID™, AND TEHO-42™)). Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (for example, but not limited to, COX-1 and/or COX-2). Examples of steroidal anti-inflammatory drugs include, but are not limited to, glucocorticoids, dexamethasone (DECADRON™), corticosteroids (for example, but not limited to, methylprednisolone (MEDROL™)), cortisone, hydrocortisone, prednisone (PREDNISONE™ and DELTASONE™), prednisolone (PRELONE™ and PEDIAPRED™), triamcinolone, azulfidine, and inhibitors of eicosanoids (for example, but not limited to, prostaglandins, thromboxanes, and leukotrienes.

In one embodiment, an effective amount of one or more antibody formulations of the invention is administered in combination with a mast cell protease inhibitor to a subject at risk of or with an inflammatory disorder. In another embodiment, the mast cell protease inhibitor is a tryptase kinase inhibitor, such as, but not limited to GW-45, GW-58, and genisteine. In a specific embodiment, the mast cell protease inhibitor is phosphatidylinositide-3′ (PI3)-kinase inhibitors, such as, but not limited to calphostin C. In another embodiment, the mast cell protease inhibitor is a protein kinase inhibitor such as, but not limited to staurosporine. In accordance with this embodiments, the mast cell protease inhibitor is preferably administered locally to the affected area.

Specific examples of immunomodulatory agents which can be administered in combination with a liquid formulation of the invention to a subject with an inflammatory disorder include, but are not limited to, methothrexate, leflunomide, cyclophosphamide, cytoxan, Immuran, cyclosporine A, minocycline, azathioprine, antibiotics (for example, but not limited to, FK506 (tacrolimus)), methylprednisolone (MP), corticosteroids, steroids, mycophenolate mofetil, rapamycin (sirolimus), mizoribine, deoxyspergualin, brequinar, malononitriloamindes (for example, but not limited to, leflunamide), anti-T cell receptor antibodies (for example, but not limited to, anti-CD4 antibodies (for example, but not limited to, cM-T412 (Boeringer), IDEC-CE9.1 (IDEC and SKB), mAB 4162W94, Orthoclone and OKTcdr4a (Janssen-Cilag)), anti-CD3 antibodies (for example, but not limited to, Nuvion (Product Design Labs), OKT3 (Johnson & Johnson), or Rituxan (IDEC)), anti-CD5 antibodies (for example, but not limited to, an anti-CD5 ricin-linked immunoconjugate), anti-CD7 antibodies (for example, but not limited to, CHH-380 (Novartis)), anti-CD8 antibodies, anti-CD40 ligand monoclonal antibodies (for example, but not limited to, IDEC-131 (IDEC)), anti-CD52 antibodies (for example, but not limited to, CAMPATH 1H (Ilex)), anti-CD2 antibodies (for example, but not limited to, MEDI-507 (MedImmune, Inc., International Publication Nos. WO 02/098370 and WO 02/069904), anti-CD11a antibodies (for example, but not limited to, Raptiva (Genentech)), and anti-B7 antibodies (for example, but not limited to, IDEC-114) (IDEC)); anti-cytokine receptor antibodies (for example, but not limited to, anti-IFN receptor antibodies, anti-IL-2 receptor antibodies (for example, but not limited to, Zenapax (Protein Design Labs)), anti-IL-4 receptor antibodies, anti-IL-6 receptor antibodies, anti-IL-10 receptor antibodies, and anti-IL-12 receptor antibodies), anti-cytokine antibodies (for example, but not limited to, anti-IFN antibodies, anti-TNF-α antibodies, anti-IL-1β antibodies, anti-IL-6 antibodies, anti-IL-8 antibodies (for example, but not limited to, ABX-IL-8 (Abgenix)), and anti-IL-12 antibodies)); CTLA4-immunoglobulin; LFA-3TIP (Biogen, International Publication No. WO 93/08656 and U.S. Pat. No. 6,162,432); soluble cytokine receptors (for example, but not limited to, the extracellular domain of a TNF-α receptor or a fragment thereof, the extracellular domain of an IL-1β receptor or a fragment thereof, and the extracellular domain of an IL-6 receptor or a fragment thereof); cytokines or fragments thereof (for example, but not limited to, interleukin (IL)-2, IL-3, IL-4, IL-5, IL-6, IL-7, IL-8, IL-9, IL-10, IL-11, IL-12, IL-15, TNF-α, TNF-β, interferon (IFN)-α, IFN-β, IFN-γ, and GM-CSF); and anti-cytokine antibodies (for example, but not limited to, anti-IL-2 antibodies, anti-IL-4 antibodies, anti-IL-6 antibodies, anti-IL-10 antibodies, anti-IL-12 antibodies, anti-IL-15 antibodies, anti-TNF-α antibodies, and anti-IFN-γ antibodies).

Any TNF-α antagonist well-known to one of skill in the art can be used in the compositions and methods of the invention. Non-limiting examples of TNF-α antagonists which can be administered in combination with a liquid formulation of the invention to a subject with an inflammatory disorder include proteins, polypeptides, peptides, fusion proteins, antibodies (for example, but not limited to, human, humanized, chimeric, monoclonal, polyclonal, Fvs, ScFvs, Fab fragments, F(ab)₂ fragments, and antigen-binding fragments thereof) such as antibodies that specifically bind to TNF-α, nucleic acid molecules (for example, but not limited to, antisense molecules or triple helices), organic molecules, inorganic molecules, and small molecules that blocks, reduces, inhibits or neutralizes the function, activity and/or expression of TNF-α. In various embodiments, a TNF-α antagonist reduces the function, activity and/or expression of TNF-α by at least 10%, at least 15%, at least 20%, at least 25%, at least 30%, at least 35%, at least 40%, at least 45%, at least ⁵⁰%, at least 55%, at least 60%, at least 65%, at least 70%, at least 75%, at least 80%, at least 85%, at least 90%, at least 95% or at least 99% relative to a control such as phosphate buffered saline (PBS). Examples of antibodies that specifically bind to TNF-α include, but are not limited to, infliximab (REMICADE™; Centacor), D2E7 (Abbott Laboratories/Knoll Pharmaceuticals Co., Mt. Olive, N.J.), CDP571 which is also known as HUMICADE™ and CDP-870 (both of Celltech/Pharmacia, Slough, U.K.), and TN3-19.12 (Williams et al., 1994, Proc. Natl. Acad. Sci. USA 91: 2762-2766; Thorbecke et al., 1992, Proc. Natl. Acad. Sci. USA 89:7375-7379). The present invention also encompasses the use of antibodies that specifically bind to TNF-α disclosed in the following U.S. patents in the compositions and methods of the invention: U.S. Pat. Nos. 5,136,021; 5,147,638; 5,223,395; 5,231,024; 5,334,380; 5,360,716; 5,426,181; 5,436,154; 5,610,279; 5,644,034; 5,656,272; 5,658,746; 5,698,195; 5,736,138; 5,741,488; 5,808,029; 5,919,452; 5,958,412; 5,959,087; 5,968,741; 5,994,510; 6,036,978; 6,114,517; and 6,171,787; each of which are herein incorporated by reference in their entirety. Examples of soluble TNF-α receptors include, but are not limited to, sTNF-R1 (Amgen), etanercept (ENBREL™; Immunex) and its rat homolog RENBREL™, soluble inhibitors of TNF-α derived from TNFrI, TNFrII (Kohno et al., 1990, Proc. Natl. Acad. Sci. USA 87:8331-8335), and TNF-α Tnh (Seckinger et al, 1990, Proc. Natl. Acad. Sci. USA 87:5188-5192).

Other TNF-α antagonists encompassed by the invention include, but are not limited to, IL-10, which is known to block TNF-α production via interferon γ-activated macrophages (Oswald et al. 1992, Proc. Natl. Acad. Sci. USA 89:8676-8680), TNFR-IgG (Ashkenazi et al., 1991, Proc. Natl. Acad. Sci. USA 88:10535-10539), the murine product TBP-1 (Serono/Yeda), the vaccine CytoTAb (Protherics), antisense molecule104838 (ISIS), the peptide RDP-58 (SangStat), thalidomide (Celgene), CDC-801 (Celgene), DPC-333 (Dupont), VX-745 (Vertex), AGIX-4207 (AtheroGenics), ITF-2357 (Italfarmaco), NPI-13021-31 (Nereus), SCIO-469 (Scios), TACE targeter (Immunix/AHP), CLX-120500 (Calyx), Thiazolopyrim (Dynavax), auranofin (Ridaura) (SmithKline Beecham Pharmaceuticals), quinacrine (mepacrine dichlorohydrate), tenidap (Enablex), Melanin (Large Scale Biological), and anti-p38 MAPK agents by Uriach.

Non-limiting examples of anti-inflammatory agents which can be administered in combination with a liquid formulation of the invention to a subject with an inflammatory disorder include non-steroidal anti-inflammatory drugs (NSAIDs), steroidal anti-inflammatory drugs, beta-agonists, anticholingeric agents, and methyl xanthines. Examples of NSAIDs include, but are not limited to, aspirin, ibuprofen, celecoxib (CELEBREX™), diclofenac (VOLTAREN™), etodolac (LODINE™), fenoprofen (NALFON™), indomethacin (INDOCIN™), ketoralac (TORADOL™), oxaprozin (DAYPRO™), nabumentone (RELAFEN™), sulindac (CLINORIL™), tolmentin (TOLECTIN™), rofecoxib (VIOXX™), naproxen (ALEVE™, NAPROSYN™), ketoprofen (ACTRON™) and nabumetone (RELAFEN™). Such NSAIDs function by inhibiting a cyclooxgenase enzyme (for example, but not limited to, COX-1 and/or COX-2). Examples of steroidal anti-inflammatory drugs include, but are not limited to, glucocorticoids, dexamethasone (DECADRON™), cortisone, hydrocortisone, prednisone (DELTASONE™), prednisolone, triamcinolone, azulfidine, and eicosanoids such as prostaglandins, thromboxanes, and leukotrienes.

### 5.7.2. Autoimmune Disorder Treatment

The liquid formulations of the invention may be administered to a subject in need thereof to prevent, treat and/or manage an autoimmune disorder or one or more symptoms thereof. The liquid formulations of the invention may also be administered in combination with one or more other therapies, preferably therapies useful for the prevention, management or treatment of an autoimmune disorder to a subject in need thereof to prevent, treat and/or manage an autoimmune disorder or one or more symptoms thereof. In a specific embodiment, the invention provides a method of preventing, treating and/or managing an autoimmune disorder or one or more symptoms thereof, said method comprising administering to a subject in need thereof a dose of a prophylactically or therapeutically effective amount of a liquid formulation of the invention. In another embodiment, the invention provides a method of preventing, treating and/or managing an autoimmune disorder or one or more symptoms thereof, said method comprising administering to a subject in need thereof a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that specifically bind to aninterferon alpha polypeptide.

The invention provides methods for preventing, treating and/or managing an autoimmune disorder or one or more symptoms thereof in a subject refractory to conventional therapies for such an autoimmune disorder, said methods comprising administering to said subject a dose of a prophylactically or therapeutically effective amount of a liquid formulation of the invention. The invention also provides methods for preventing, treating and/or managing an autoimmune disorder or one or more symptoms thereof in a subject refractory to existing single agent therapies for such an autoimmune disorder, said methods comprising administering to said subject a dose of a prophylactically or therapeutically effective amount of a liquid formulation of the invention and a dose of a prophylactically or therapeutically effective amount of one or more therapies (e.g., prophylactic or therapeutic agents) other than antibodies (including antibody fragments thereof) that specifically bind to an interferon alpha polypeptide. The invention also provides methods for preventing, treating and/or managing an autoimmune disorder or one or more symptoms thereof by administering a liquid formulation of the invention in combination with any other treatment to patients who have proven refractory to other treatments but are no longer on these treatments. The invention also provides alternative methods for the management or treatment of an autoimmune disorder where another therapy has proven or may prove too toxic, i.e., results in unacceptable or unbearable side effects, for the subject being treated. Particularly, the invention provides alternative methods for the management or treatment of an autoimmune disorder where the patient is refractory to other therapies. Further, the invention provides methods for preventing the recurrence of an autoimmune disorder in patients that have been treated and have no disease activity by administering a liquid formulation of the invention.

In autoimmune disorders, the immune system triggers an immune response when there are no foreign substances to fight and the body's normally protective immune system causes damage to its own tissues by mistakenly attacking self. There are many different autoimmune disorders which affect the body in different ways. For example, the brain is affected in individuals with multiple sclerosis, the gut is affected in individuals with Crohn's disease, and the synovium, bone and cartilage of various joints are affected in individuals with rheumatoid arthritis. As autoimmune disorders progress destruction of one or more types of body tissues, abnormal growth of an organ, or changes in organ function may result. The autoimmune disorder may affect only one organ or tissue type or may affect multiple organs and tissues. Organs and tissues commonly affected by autoimmune disorders include red blood cells, blood vessels, connective tissues, endocrine glands (for example, but not limited to, the thyroid or pancreas), muscles, joints, and skin. Examples of autoimmune disorders that can be treated by the methods of the invention include, but are not limited to, alopecia areata, ankylosing spondylitis, antiphospholipid syndrome, autoimmune Addison's disease, autoimmune diseases of the adrenal gland, autoimmune hemolytic anemia, autoimmune hepatitis, autoimmune oophoritis and orchitis, autoimmune thrombocytopenia, Behçet's disease, bullous pemphigoid, cardiomyopathy, celiac sprue-dermatitis, chronic fatigue immune dysfunction syndrome (CFIDS), chronic inflammatory demyelinating polyneuropathy, Churg-Strauss syndrome, cicatrical pemphigoid, CREST syndrome, cold agglutinin disease, Crohn's disease, discoid lupus, essential mixed cryoglobulinemia, fibromyalgia-fibromyositis, glomerulonephritis, Graves' disease, Guillain-Barre, Hashimoto's thyroiditis, idiopathic pulmonary fibrosis, idiopathic thrombocytopenia purpura (ITP), IgA neuropathy, juvenile arthritis, lichen planus, lupus erthematosus, Ménière's disease, mixed connective tissue disease, multiple sclerosis, type 1 or immune-mediated diabetes mellitus, myasthenia gravis, pemphigus vulgaris, pernicious anemia, polyarteritis nodosa, polychrondritis, polyglandular syndromes, polymyalgia rheumatica, polymyositis and dermatomyositis, primary agammaglobulinemia, primary biliary cirrhosis, psoriasis, psoriatic arthritis, Raynauld's phenomenon, Reiter's syndrome, Rheumatoid arthritis, sarcoidosis, scleroderma, Sjögren's syndrome, stiff-man syndrome, systemic lupus erythematosus, lupus erythematosus, takayasu arteritis, temporal arteristis/giant cell arteritis, ulcerative colitis, uveitis, vasculitides such as dermatitis herpetiformis vasculitis, vitiligo, Wegener's granulomatosis, idiopathic inflammatory myopathies (IIM), dermatomyositis (DM), polymyositis (PM), and inclusion body myositis (IBM).

Autoimmune therapies and their dosages, routes of administration and recommended usage are known in the art and have been described in such literature as the Physicians' Desk Reference (60th ed., 2006).

### 5.7.2.1. Autoimmune Disorder Therapies

The present invention provides methods of preventing, treating and/or managing an autoimmune disorder or one or more symptoms thereof, said methods comprising administering to a subject in need thereof a liquid formulation of the invention and one or more therapies (e.g., prophylactic or therapeutic agents) other than antibodies (including antibody fragments thereof) that specifically bind to an interferon alpha polypeptide. Any agent or therapy which is known to be useful, or which has been used or is currently being used for the prevention, treatment and/or management of an autoimmune disorder or one or more symptoms thereof can be used in combination with a liquid formulation of the invention in accordance with the invention described herein. Examples of such agents include, but are not limited to, immunomodulatory agents, anti-inflammatory agents and TNF-α antagonists.

In specific embodiments, patients with multiple sclerosis (MS) are administered a prophylactically or therapeutically effective amount of a liquid formulation of the invention in combination with other agents or therapies useful in prevention, treatment and/or management of MS including but not limited to: IFN-β1b (Betaseron) (e.g., 8.0 million international unites (MIU) is administered by subcutaneous injection every other day); IFN-β1a (Avonex) (e.g., 6.0 MIU is administered by intramuscular injection once every week); glatiramer acetate (Copaxone) (e.g., 20 mg is administered by subcutaneous injection every day); mitoxantrone (e.g., 12 mg/m² is administered by intravenous infusion every third month); azathioprine (e.g., 2-3 mg/kg body weight is administered orally each day); methotrexate (e.g., 7.5 mg is administered orally once each week); cyclophosphamide; intravenous immunoglobulin (e.g., 0.15-0.2 g/kg body weight administered monthly for up to 2 years); glucocorticoids; methylprednisolone (e.g., administered in bimonthly cycles at high doses); 2-chlorodeoxyadenosine (cladribine); baclofen (e.g., 15 to 80 mg/d in divided doses, or orally in higher doses up to 240 mg/d, or intrathecally via an indwelling catheter); cycloenzaprine hydrochloride (e.g., 5-10 mg bid or tid); clonazepam (e.g., 0.5 to 1.0 mg tid, including bedtime dose); clonidine hydrochloride (e.g., 0.1 to 0.2 mg tid, including a bedtime dose); carbamazepine (e.g., 100-1200 mg/d in divided, escalating doses); gabapentin (e.g., 300-3600 mg/d); dilantin (e.g., 300-400 mg/d); amitriptyline (e.g., 25-150 mg/d); baclofen (e.g., 10-80 mg/d); primidone (e.g., 125-250 mg bid or tid); ondansetron (e.g., 4 to 8 mg bid or tid); isoniazid (e.g., up to 1200 mg in divided doses); oxybutynin (e.g., 5 mg bid or tid); tolterodine (e.g., 1-2 mg bid); propantheline (e.g., 7.5 to 15 mg qid); bethanecol (e.g., 10-50 mg tid or qid); terazosin hydrochloride (e.g., 1-5 mg at bedtime); sildenafil citrate (e.g., 50-100 mg po prn); amantading (e.g., 100 mg bid); pemoline (e.g., 37.5 mg bid); high dose vitamins; calcium orotate; gancyclovir; antibiotic; and plasma exchange.

In specific embodiments, patients with psoriasis are administered a prophylactically or therapeutically effective amount of a liquid formulation of the invention in combination with other agents or therapies useful in prevention, treatment and/or management of psoriasis including but not limited to: topical steroid cream or ointment; tar (examples including but not limited to, Estar, Psorigel, Fototar cream, and LCD 10% in Nutraderm lotion or mixed directly with triamcinolone 0.1% cream); occlusion; topical vitamin D analogue (a non-limiting example is calcipotriene ointment); ultraviolet light; PUVA (psoralen plus ultraviolet A); methotrexate (e.g., up to 25 mg once weekly or in divided doses every 12 hours for three doses once a week); synthetic retinoid (a non-limiting examples is etretinate, e.g., in dosage of 0.5-1 mg/kg/d); immunomodulatory therapy (a non-limiting example is cyclosporine); sulfasalazine (e.g., in dosages of 1 g three times daily).

In specific embodiments, patients with Crohn's disease are administered a prophylactically or therapeutically effective amount of a liquid formulation of the invention in combination with other agents or therapies useful in prevention, treatment and/or management of Crohn's disease including but not limited to: antidiarrheals (e.g., loperamide 2-4 mg up to 4 times a day, diphenoxylate with atropine 1 tablet up to 4 times a day, tincture of opium 8-15 drops up to 4 times a day, cholestyramine 2-4 g or colestipol 5 g once or twice daily), antispasmodics (e.g., propantheline 15 mg, dicyclomine 10-20 mg, or hyoscyamine 0.125 mg given before meals), 5-aminosalicylic acid agents (e.g., sulfasalazine 1.5-2 g twice daily, mesalamine (ASACOL®) and its slow release form (PENTASA®), especially at high dosages, e.g., PENTASA® 1 g four times daily and ASACOL® 0.8-1.2 g four times daily), corticosteroids, immunomodulatory drugs (e.g., azathioprine (1-2 mg/kg), mercaptopurine (50-100 mg), cyclosporine, and methotrexate), antibiotics, TNF inhibitors (e.g., inflixmab (REMICADE®)), immunosuppressive agents (e.g., tacrolimus, mycophenolate mofetil, and thalidomide), anti-inflammatory cytokines (for example, but not limited to, IL-10 and IL-11), nutritional therapies, enteral therapy with elemental diets (e.g., Vivonex for 4 weeks), and total parenteral nutrition.

In specific embodiments, patients with lupus erythematosus are administered a prophylactically or therapeutically effective amount of a liquid formulation of the invention in combination with other agents or therapies useful in prevention, treatment and/or management of lupus erythematosus including but not limited to: antimalarials (including but not limited to, hydroxychloroquine); glucocorticoids (for example, but not limited to, low dose, high dose, or high-dose intravenous pulse therapy can be used); immunosuppressive agents (including but not limited to, cyclophosphamide, chlorambucil, and azanthioprine); cytotoxic agents (including but not limited to methotrexate and mycophenolate mofetil); androgenic steroids (including but not limited to danazol); and anticoagulants (including but not limited to warfarin).

The antibody formulations of the invention or combination therapies of the invention may be used as the first, second, third, fourth, or fifth therapy to prevent, treat and/or manage an autoimmune disorder or one or more symptom thereof. The invention also includes methods of preventing, treating and/or managing an autoimmune disorder or one or more symptoms thereof in a patient undergoing therapies for other disease or disorders. The invention encompasses methods of preventing, treating and/or managing an autoimmune disorder or one or more symptoms thereof in a patient before any adverse effects or intolerance to therapies other than antibodies of the invention develops. The invention also encompasses methods of preventing, treating and/or managing an autoimmune disorder or a symptom thereof in refractory patients. The invention encompasses methods for preventing, treating and/or managing a proliferative disorder or a symptom thereof in a patient who has proven refractory to therapies other than antibodies, compositions, or combination therapies of the invention. The determination of whether a patient is refractory can be made either in vivo or in vitro by any method known in the art for assaying the effectiveness of a treatment of autoimmune disorders, using art-accepted meanings of “refractory” such a context. In certain embodiments, a patent with an autoimmune disorder is refractory to a therapy when one or more symptoms of an autoimmune disorder is not prevented, managed, and/or alleviated. The invention also encompasses methods of preventing, treating and/or managing an autoimmune disorder or a symptom thereof in patients who are susceptible to adverse reactions to conventional therapies.

The present invention encompasses methods for preventing, treating and/or managing an autoimmune disorder or one or more symptoms thereof as an alternative to other conventional therapies. In specific embodiments, the patient being managed or treated in accordance with the methods of the invention is refractory to other therapies or is susceptible to adverse reactions from such therapies. The patient may be a person with a suppressed immune system (for example, but not limited to, post-operative patients, chemotherapy patients, and patients with immunodeficiency disease, patients with broncho-pulmonary dysplasia, patients with congenital heart disease, patients with cystic fibrosis, patients with acquired or congenital heart disease, and patients suffering from an infection), a person with impaired renal or liver function, the elderly, children, infants, infants born prematurely, persons with neuropsychiatric disorders or those who take psychotropic drugs, persons with histories of seizures, or persons on medication that would negatively interact with conventional agents used to prevent, treat and/or manage a viral respiratory infection or one or more symptoms thereof.

Autoimmune therapies and their dosages, routes of administration and recommended usage are known in the art and have been described in such literature as the Physicians' Desk Reference (60th ed., 2006).

### 5.8. Methods of Administering the Antibody Formulations

The invention provides methods of prevention, treatment and/or management of a disorder, for example, a disorder associated with or characterized by aberrant expression and/or activity of, e.g., an interferon alpha polypeptide, a disorder associated with aberrant expression and/or activity of an interferon alpha receptor or one or more subunits thereof, an autoimmune disorder, an inflammatory disorder, a proliferative disorder, an infection, or one or more symptoms thereof by administrating to a subject of an effective amount of liquid formulations of the invention. Various delivery systems are known and can be used to administer a liquid formulation of the present invention or a prophylactic or therapeutic agent. Methods of administering antibody liquid formulations of the present invention or a therapy (e.g., a prophylactic or therapeutic agent) include, but are not limited to, parenteral administration (e.g., intradermal, intramuscular, intraperitoneal, intravenous and, and subcutaneous), epidural administration, topical administration, and mucosal administration (for example, but not limited to, intranasal and oral routes). In a specific embodiment, liquid formulations of the present invention are administered intramuscularly, intravenously, or subcutaneously. In one embodiment, the liquid formulations of the invention are administered subcutaneously. The formulations may be administered by any convenient route, for example by infusion or bolus injection, by absorption through epithelial or mucocutaneous linings (e.g., oral mucosa, rectal and intestinal mucosa, etc.) and may be administered together with other biologically active agents. Administration can be systemic or local.

The invention also provides that a liquid formulation of the present invention is packaged in a hermetically sealed container such as an ampoule or sachette indicating the quantity of antibody (including antibody fragment thereof). In one embodiment, a liquid formulation of the present invention is in a hermetically sealed container indicating the quantity and concentration of the antibody (including antibody fragment thereof). In one embodiment, a liquid formulation of the present invention is supplied in a hermetically sealed container and comprises about 10 mg/ml, about 15 mg/ml, about 20 mg/ml, about 30 mg/ml, about 40 mg/ml, about 50 mg/ml, about 60 mg/ml, about 70 mg/ml, about 80 mg/ml, about 90 mg/ml, about 100 mg/ml, about 150 mg/ml, about 175 mg/ml, about 200 mg/ml, about 250 mg/ml, or about 300 mg/ml of an antibody (including antibody fragment thereof) that specifically binds to an interferon alpha polypeptide, in a quantity of about 1 ml, about 2 ml, about 3 ml, about 4 ml, about 5 ml, 6 about ml, about 7 ml, about 8 ml, about 9 ml, about 10 ml, about 15 ml, or about 20 ml. In a specific embodiment of the invention, a liquid formulation of the invention is supplied in a hermetically sealed container and comprises at least about 15 mg/ml, at least about 20 mg/ml, at least about 25 mg/ml, at least about 50 mg/ml, at least about 100 mg/ml, at least about 150 mg/ml, at least about 175 mg/ml, at least about 200 mg/ml, at least about 250 mg/ml or at least about 300 mg/ml of an antibody (including antibody fragment thereof) that specifically binds to an interferon alpha polypeptide (for example, but not limited to, 13H5 or an antigen-binding fragment thereof) for intravenous injections, and at least about 15 mg/ml, at least about 20 mg/ml, at least about 50 mg/ml, at least about 80 mg/ml, at least about 100 mg/ml, at least about 150 mg/ml, at least about 175 mg/ml, at least about 200 mg/ml, at least about 250 mg/ml or at least about 300 mg/ml of an antibody (including antibody fragment thereof) that specifically binds to an interferon alpha polypeptide (for example, but not limited to, 13H5 or a fragment thereof) for repeated subcutaneous administration.

The amount of a liquid formulation of the present invention which will be effective in the prevention, treatment and/or management of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof can be determined by standard clinical techniques well-known in the art or described herein. The precise dose to be employed in the formulation will also depend on the route of administration, and the seriousness of the inflammatory disorder, or autoimmune disorder, and should be decided according to the judgment of the practitioner and each patient's circumstances. Effective doses may be extrapolated from dose-response curves derived from in vitro or animal model test systems.

For formulations of the antibodies, proteins, polypeptides, peptides and fusion proteins encompassed by the invention, the dosage administered to a patient may be calculated using the patient's weight in kilograms (kg) multiplied by the dose to be administered in mg/kg. The required volume (in mL) to be given is then determined by taking the mg dose required divided by the concentration of the antibody formulation. The final calculated required volume will be obtained by pooling the contents of as many vials as are necessary into syringe(s) to administer the antibody formulation of the invention. The final calculated required volume will be obtained by pooling the contents of as many vials as are necessary into syringe(s) to administer the drug. A maximum volume of 2.0 mL of the antibody formulation can be injected per site. The dose (in mL) can be calculated using the following formula: Dose (mL)=[volunteer weight](kg)×[dose]mg/kg÷100 mg/mL of the antibody formulation. Generally, human antibodies have a longer half-life within the human body than antibodies from other species due to the immune response to the foreign polypeptides. Thus, lower dosages of human antibodies and less frequent administration is often possible. Further, the dosage, volume and frequency of administration of liquid formulations of the present invention may be reduced by increasing the concentration of an antibody (including antibody fragment thereof) in the formulations, increasing affinity and/or avidity of the antibody (including antibody fragment thereof), and/or increasing the half-life of the antibody (including antibody fragment thereof).

In a specific embodiment, the dosage administered to a patient will be calculated using the patient's weight in kilograms (kg) multiplied by the dose to be administered in mg/kg. The required volume (in mL) to be given is then determined by taking the mg dose required divided by the concentration of the antibody (including antibody fragment thereof) in the formulations (100 mg/mL). The final calculated required volume will be obtained by pooling the contents of as many vials as are necessary into syringe(s) to administer the drug. A maximum volume of 2.0 mL of antibody (including antibody fragment thereof) in the formulations can be injected per site.

In a specific embodiment, 0.1 to 20 mg/kg/week, 1 to 15 mg/kg/week, 2 to 8 mg/week, 3 to 7 mg/kg/week, or 4 to 6 mg/kg/week of an antibody (including antibody fragment thereof) that specifically binds to an interferon alpha polypeptide (for example, but not limited to, 13H5 or a fragment thereof) in a liquid formulation of the invention is administered to a subject with an inflammatory disorder or an autoimmune disorder. In another embodiment, a subject is administered one or more doses of a prophylactically or therapeutically effective amount of a liquid formulation of the invention, wherein the prophylactically or therapeutically effective amount is not the same for each dose.

In one embodiment, a liquid formulation of the invention is administered in a dosing regimen that maintains the plasma concentration of the antibody specific for interferon alpha at a desirable level (e.g., from about 0.1 to about 100 μg/ml), which continuously blocks the interferon alpha polypeptide activity. In a specific embodiment, the plasma concentration of the antibody is maintained at about 0.2 μg/ml, about 0.5 μg/ml, about 1 μg/ml, about 2 μg/ml, about 3 μg/ml, about 4 μg/ml, about 5 μg/ml, about 6 μg/ml, about 7 μg/ml, about 8 μg/ml, about 9 μg/ml, about 10 μg/ml, about 15 μg/ml, about 20 μg/ml, about 25 μg/ml, about 30 μg/ml, about 35 μg/ml, about 40 μg/ml, about 45 μg/ml or about 50 μg/ml. The plasma concentration that is desirable in a subject will vary depending on several factors, including but not limited to, the nature of the disease or disorder, the severity of the disease or disorder and the condition of the subject. Such dosing regimens are especially beneficial in prevention, treatment and/or management of a chronic disease or disorder.

In specific embodiments, a liquid formulation of the invention comprising a conjugated antibody (including antibody fragment thereof) specific for an interferon alpha polypeptide is administered intermittently. As used herein, “a conjugated antibody or antibody fragment” refers to an antibody (including antibody fragment thereof) that is conjugated or fused to another moiety, including but not limited to, a heterologous peptide, polypeptide, another antibody (including antibody fragment thereof), a marker sequence, a diagnostic agent, a polymer, albumin, and a solid support.

In another embodiment, a human subject is administered one or more doses of a prophylactically or therapeutically effective amount of an antibody (including antibody fragment thereof) that specifically binds to an interferon alpha polypeptide (for example, but not limited to, 13H5 or a fragment thereof) in a liquid formulation of the invention, wherein the dose of a prophylactically or therapeutically effective amount of the antibody (including antibody fragment thereof) in the liquid formulation of the invention administered to said subject is increased by, e.g., about 0.01 μg/kg, about 0.02 μg/kg, about 0.04 μg/kg, about 0.05 μg/kg, about 0.06 μg/kg, about 0.08 μg/kg, about 0.1 μg/kg, about 0.2 μg/kg, about 0.25 μg/kg, about 0.5 μg/kg, about 0.75 μg/kg, about 1 μg/kg, about 1.5 μg/kg, about 2 μg/kg, about 4 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μg/kg, about 70 μg/kg, about 75 μg/kg, about 80 μg/kg, about 85 μg/kg, about 90 μg/kg, about 95 μg/kg, about 100 μg/kg, or about 125 μg/kg, as treatment progresses.

In another embodiment, a subject (e.g., a human) is administered one or more doses of a prophylactically or therapeutically effective amount of an antibody (including antibody fragment thereof) that specifically binds to an interferon alpha polypeptide (for example, but not limited to, 13H5 or a fragment thereof) in a liquid formulation of the invention, wherein the dose of a prophylactically or therapeutically effective amount of the antibody (including antibody fragment thereof) in the liquid formulation of the invention administered to said subject is decreased by, e.g., about 0.01 μg/kg, about 0.02 μg/kg, about 0.04 μg/kg, about 0.05 μg/kg, about 0.06 μg/kg, about 0.08 μg/kg, about 0.1 μg/kg, about 0.2 μg/kg, about 0.25 μg/kg, about 0.5 μg/kg, about 0.75 μg/kg, about 1 μg/kg, about 1.5 μg/kg, about 2 μg/kg, about 4 μg/kg, about 5 μg/kg, about 10 μg/kg, about 15 μg/kg, about 20 μg/kg, about 25 μg/kg, about 30 μg/kg, about 35 μg/kg, about 40 μg/kg, about 45 μg/kg, about 50 μg/kg, about 55 μg/kg, about 60 μg/kg, about 65 μi/kg, about 70 μg/kg, about 75 μg/kg, about 80 μg/kg, about 85 μg/kg, about 90 μg/kg, about 95 μi/kg, about 100 μg/kg, or about 125 μg/kg, as treatment progresses.

The dosages of prophylactic or therapeutic agents are described in the Physicians' Desk Reference (60th ed., 2006).

### 5.9. Antibody Characterization

The antibodies (including antibody fragment thereof) of the liquid formulations of the invention may be characterized in a variety of ways well-known to one of skill in the art. For example, antibodies (including antibody fragments thereof) of the liquid formulations of the invention may be assayed for the ability to specifically bind to antigen. Such an assay may be performed in solution (e.g., Houghten, 1992, Bio/Techniques 13:412-421), on beads (Lam, 1991, Nature 354:82-84), on chips (Fodor, 1993, Nature 364:555-556), on bacteria (U.S. Pat. No. 5,223,409), on spores (U.S. Pat. Nos. 5,571,698; 5,403,484; and 5,223,409), on plasmids (Cull et al., 1992, Proc. Natl. Acad. Sci. USA 89:1865-1869) or on phage (Scott and Smith, 1990, Science 249:386-390; Cwirla et al., 1990, Proc. Natl. Acad. Sci. USA 87:6378-6382; and Felici, 1991, J. Mol. Biol. 222:301-310) (each of these references is incorporated herein in its entirety by reference). For example, antibodies (including antibody fragments thereof) that have been identified to specifically bind to an interferon alpha polypeptide can then be assayed for their specificity and affinity for an interferon alpha polypeptide.

The antibodies (including antibody fragments thereof) of the liquid formulations of the invention may be assayed for specific binding to antigen and cross-reactivity with other antigens by any method known in the art. Immunoassays which can be used to analyze specific binding and cross-reactivity include, but are not limited to, competitive and non-competitive assay systems using techniques such as western blots, radioimmunoassays, ELISA (enzyme linked immunosorbent assay), “sandwich” immunoassays, immunoprecipitation assays, precipitin reactions, gel diffusion precipitin reactions, immunodiffusion assays, agglutination assays, complement-fixation assays, immunoradiometric assays, fluorescent immunoassays, protein A immunoassays, to name but a few. Such assays are routine and well known in the art (see, e.g., Ausubel et al., eds., 1994, Current Protocols in Molecular Biology, Vol. 1, John Wiley & Sons, Inc., New York, which is incorporated by reference herein in its entirety).

The binding affinity of an antibody to an antigen and the off-rate of an antibody-antigen interaction can be determined by competitive binding assays. One example of a competitive binding assay is a radioimmunoassay comprising the incubation of labeled antigen (for example, but not limited to, ³H or ¹²⁵I) with the antibody of interest in the presence of increasing amounts of unlabeled antigen, and the detection of the antibody bound to the labeled antigen. The affinity of the antibody of the contained in a liquid formulation of the present invention or a fragment thereof for a specific antigen and the binding off-rates can be determined from the data by scatchard plot analysis. Competition with a second antibody can also be determined using radioimmunoassays. In one example, an interferon alpha polypeptide is incubated with an antibody conjugated to a labeled compound (for example, but not limited to, ³H or ¹²⁵I) in the presence of increasing amounts of an unlabeled second antibody.

If the antibodies (including antibody fragments thereof) of the liquid formulations of the invention are specific for a ligand, the antibodies can also be assayed for their ability to inhibit the binding of the ligand to its receptor using techniques known to those of skill in the art. In a specific embodiment, the ability of antibodies (including antibody fragments thereof) of the liquid formulations of the invention to inhibit ligand binding to its receptor can be measured by cell proliferation assays.

The antibodies (including antibody fragments thereof) of the liquid formulations of the invention can be tested for binding to IFN alpha by, for example, standard ELISA or by Biacore analysis. Briefly, for ELISAs, microtiter plates are coated with IFN alpha (e.g., the recombinant form of different IFN alpha subtypes, or leukocyte or lymphoblastoid IFN) at 0.25 μg/ml in PBS, and then blocked with 5% bovine serum albumin in PBS. Dilutions of antibody (e.g., dilutions of plasma from IFN alpha-immunized mice) are added to each well and incubated for 1-2 hours at 37° C. The plates are washed with PBS/Tween and then incubated with secondary reagent (e.g., for human antibodies, a goat-anti-human IgG Fc-specific polyclonal reagent) conjugated to alkaline phosphatase for 1 hour at 37° C. After washing, the plates are developed with pNPP substrate (1 mg/ml), and analyzed at OD of 405-650. Mice which develop the highest titers may be used for fusions.

To determine if the selected anti-IFN alpha monoclonal antibodies bind to unique epitopes, each antibody can be biotinylated using commercially available reagents (Pierce, Rockford, Ill.). Competition studies using unlabeled monoclonal antibodies and biotinylated monoclonal antibodies can be performed using IFN alpha coated-ELISA plates as described above. Biotinylated mAb binding can be detected with a strep-avidin-alkaline phosphatase probe.

To determine the isotype of purified antibodies, isotype ELISAs can be performed using reagents specific for antibodies of a particular isotype. For example, to determine the isotype of a human monoclonal antibody, wells of microtiter plates can be coated with 1 μg/ml of anti-human immunoglobulin overnight at 4° C. After blocking with 1% BSA, the plates are reacted with 1 μg/ml or less of test monoclonal antibodies or purified isotype controls, at ambient temperature for one to two hours. The wells can then be reacted with either human IgG1 or human IgM-specific alkaline phosphatase-conjugated probes. Plates are developed and analyzed as described above.

Anti-IFN alpha human IgGs can be further tested for reactivity with IFN alpha antigen by Western blotting. Briefly, cell extracts from cells expressing IFN alpha can be prepared and subjected to sodium dodecyl sulfate polyacrylamide gel electrophoresis. After electrophoresis, the separated antigens are transferred to nitrocellulose membranes, blocked with 10% fetal calf serum, and probed with the monoclonal antibodies to be tested. Human IgG binding can be detected using anti-human IgG alkaline phosphatase and developed with BCIP/NBT substrate tablets (Sigma Chem. Co., St. Louis, Mo.).

The 13H5 antibody contains a potential deamidation site at Asn-55 in the CDR2 region of the heavy chain. Deamidation of asparagines residues is a common modification of polypeptides and proteins obtained using recombinant DNA technology and may result in decreased biological activity and/or stability, though deamidation does not always correlate with loss of biological activity. Deamidation of asparagines to form aspartic acid (and iso-Asp) results in a change of net charge, which can be detected by charge-based analytical methods. To examine deamidation of 13H5 under accelerated conditions (basic pH), methods for detection of deamidated variants of Fab fragment by IEX-HPLC and capillary isoelectric focusing (cEIF) may be used (see, US Patent Publication 2007/0014724A1).

### 5.9.1. In Vivo Assays

The antibodies (including antibody fragment thereof) of the liquid formulations of the invention may be characterized in a variety of in vivo assays known to one of skill in the art. For example, interferon alpha inhibits the proliferation of Daudi (Burkitts lymphoma, ATCC #CCL-213) cells in a dose dependant manner. A neutralizing antibody, which blocks interferon binding to its receptor, will restore proliferation. Using this cell proliferation assay, the activity of human anti-IFN alpha antibodies may be assayed (see, US Patent Publication 2007/0014724A1).

Additionally, the addition of IFN alpha 2b to cell culture media is known to induce the expression of the cell surface markers CD38 and MHC Class I on normal peripheral blood mononuclear cells (PBMNC). The activity of a human anti-IFN alpha antibody may be tested for inhibition of interferon induced cell surface marker expression on cultures of primary human cells. The addition of IFN alpha 2b to cell culture media is also known to induce IP-10 expression in normal peripheral blood mononuclear cells (PBMNC). The activity of a human anti-IFN alpha antibody may be tested for inhibition of interferon induced expression of IP-10 in normal PBMNC cultures by an ELISA binding assay. For detailed description of these assays see US Patent Publication 2007/0014724A1.

SLE plasma induces dendritic cell development from normal human monocytes. Anti-IFN alpha antibodies may be tested for inhibition of dendritic cell development, as assessed by the ability of the antibodies to inhibit the induction of the cell surface markers CD38, MHC Class I and CD123 by SLE plasma (see, US Patent Publication 2007/0014724A1).

The antibodies, compositions, or combination therapies of the invention can be tested in suitable animal model systems prior to use in humans. Such animal model systems include, but are not limited to, rats, mice, chicken, cows, monkeys, pigs, dogs, rabbits, etc. Any animal system well-known in the art may be used. Several aspects of the procedure may vary; said aspects include, but are not limited to, the temporal regime of administering the therapies (e.g., prophylactic and/or therapeutic agents), whether such therapies are administered separately or as an admixture, and the frequency of administration of the therapies.

Animal models for autoimmune disorders can also be used to assess the efficacy of an antibody, a composition, or a combination therapy of the invention. Animal models for autoimmune disorders such as type 1 diabetes, thyroid autoimmunity, systemic lupus erythematosus, and glomerulonephritis have been developed (Flanders et al., 1999, Autoimmunity 29:235-246; Krogh et al., 1999, Biochimie 81:511-515; Foster, 1999, Semin. Nephrol. 19:12-24).

Further, any assays known to those skilled in the art can be used to evaluate the prophylactic and/or therapeutic utility of an antibody, a composition, a combination therapy disclosed herein for prevention, treatment, management, and/or amelioration of disease or disorder associated with or characterized by aberrant expression and/or activity of an interferon alpha polypeptide, a disease or disorder associated with or characterized by aberrant expression and/or activity of the interferon alpha receptro or one or more subunits thereof, an autoimmune disease, an autoimmune disease, transplant rejection, graft versus host disease, or one or more symptoms thereof.

### 5.9.2. Toxicity Assays

The toxicity and/or efficacy of the prophylactic and/or therapeutic protocols of the instant invention can be determined by standard pharmaceutical procedures in cell cultures or experimental animals, e.g., for determining the LD50 (the dose lethal to 50% of the population) and the ED50 (the dose therapeutically effective in 50% of the population). The dose ratio between toxic and therapeutic effects is the therapeutic index and it can be expressed as the ratio LD50/ED50. Therapies that exhibit large therapeutic indices are preferred. While therapies that exhibit toxic side effects may be used, care should be taken to design a delivery system that targets such agents to the site of affected tissue in order to minimize potential damage to uninfected cells and, thereby, reduce side effects.

The data obtained from the cell culture assays and animal studies can be used in formulating a range of dosage of the prophylactic and/or therapeutic agents for use in humans. In one embodiment, the dosage of such agents lies within a range of circulating concentrations that include the ED50 with little or no toxicity. The dosage may vary within this range depending upon the dosage form employed and the route of administration utilized. For any therapy used in the method of the invention, the therapeutically effective dose can be estimated initially from cell culture assays. A dose may be formulated in animal models to achieve a circulating plasma concentration range that includes the IC50 (i.e., the concentration of the test compound that achieves a half-maximal inhibition of symptoms) as determined in cell culture. Such information can be used to more accurately determine useful doses in humans. Levels in plasma may be measured, for example, by ELISA.

Further, any assays known to those skilled in the art can be used to evaluate the prophylactic and/or therapeutic utility of an antibody, a composition, a combination therapy disclosed herein for a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof.

### 5.10. Diagnostic Uses of Antibody Formulations

Antibodies (including molecules comprising, or alternatively consisting of, antibody fragments or variants thereof) of the liquid formulations of the invention that specifically bind to an antigen of interest (e.g., an interferon alpha polypeptide) can be used for diagnostic purposes to detect, diagnose, prognose, or monitor a disease or disorder, for example, a disease or disorder associated with or characterized by aberrant expression and/or activity of, e.g., an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof. The invention provides for the detection of aberrant expression of interferon alpha comprising: (a) assaying the expression of interferon alpha in a biological sample from an individual using one or more antibodies of the liquid formulations of the invention that specifically binds to an interferon alpha polypeptide; and (b) comparing the level of interferon alpha with a standard level of interferon alpha, e.g., in normal biological samples, whereby an increase or decrease in the assayed level of interferon alpha compared to the standard level of interferon alpha is indicative of a disease or disorder associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof. In specific embodiments, aberrant expression level of interferon alpha is indicative of an autoimmune disorder or a disease or condition associated therewith. In another specific embodiment, an aberrant expression level of interferon alpha is indicative of an inflammatory disorder or a disease or condition associated therewith, such as inflammatory bowel disease.

Antibodies of the liquid formulations of the invention can be used to assay interferon alpha levels in a biological sample using classical immunohistological methods known to those of skill in the art. Other antibody-based methods useful for detecting protein gene expression include immunoassays, such as the enzyme linked immunosorbent assay (ELISA) and the radioimmunoassay (RIA). Suitable antibody assay labels are known in the art and include enzyme labels, such as, glucose oxidase; radioisotopes, such as iodine (¹²⁵I, ¹²¹I), carbon (¹⁴C), sulfur (³⁵S), tritium (³H), indium (¹²¹In), and technetium (⁹⁹Tc); luminescent labels, such as luminol; and fluorescent labels, such as fluorescein and rhodamine, and biotin.

### 5.11. Kits

The invention provides a pharmaceutical pack or kit comprising one or more containers filled with a liquid formulation of the invention. In one embodiment, a container filled with a liquid formulation of the invention is a pre-filled syringe. In a specific embodiment, the liquid formulations of the invention comprise antibodies (including antibody fragments thereof) recombinantly fused or chemically conjugated to another moiety, including but not limited to, a heterologous protein, a heterologous polypeptide, a heterologous peptide, a large molecule, a small molecule, a marker sequence, a diagnostic or detectable agent, a therapeutic moiety, a drug moiety, a radioactive metal ion, a second antibody, and a solid support. The invention also provides a pharmaceutical pack or kit comprising in one or more first containers a liquid formulation of the invention and in one or more second containers one or more other prophylactic or therapeutic agents useful for the prevention, management or treatment of a disease or disorder, for example, a disease or disorder associated with or characterized by aberrant expression and/or activity of, e.g., an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof. In a specific embodiment, the liquid formulations of the invention are formulated in single dose vials as a sterile liquid containing 25 mM histidine buffer at pH 6.0, 8% trehalose and 0.02% Polysorbate 80. The formulations of the invention may be supplied in 3 cc USP Type I borosilicate amber vials (West Pharmaceutical Serices—Part No. 6800-0675) with a target volume of 1.2 mL. Optionally associated with such container(s) can be a notice in the form prescribed by a governmental agency regulating the manufacture, use or sale of pharmaceuticals or biological products, which notice reflects approval by the agency of manufacture, use or sale for human administration. In another embodiment, a formulation of the invention may be supplied in a pre-filled syringe.

In one embodiment, a container filled with a liquid formulation of the invention is a pre-filled syringe. Any pre-filled syringe known to one of skill in the art may be used in combination with a liquid formulation of the invention. Pre-filled syringes that may be used are described in, for example, but not limited to, PCT Publications WO05032627, WO08094984, WO9945985, WO03077976, U.S. Pat. No. 6,792,743, U.S. Pat. No. 5,607,400, U.S. Pat. No. 5,893,842, U.S. Pat. No. 7,081,107, U.S. Pat. No. 7,041,087, U.S. Pat. No. 5,989,227, U.S. Pat. No. 6,807,797, U.S. Pat. No. 6,142,976, U.S. Pat. No. 5,899,889, US Patent Publications US20070161961A1, US20050075611A1, US20070092487A1, US20040267194A1, US20060129108A1. Pre-filled syringes may be made of various materials. In one embodiment a pre-filled syringe is a glass syringe. In another embodiment a pre-filled syringe is a plastic syringe. One of skill in the art understands that the nature and/or quality of the materials used for manufacturing the syringe may influence the stability of a protein formulation stored in the syringe. For example, it is understood that silicon based lubricants deposited on the inside surface of the syringe chamber may affect particle formation in the protein formulation. In one embodiment, a pre-filled syringe comprises a silicone based lubricant. In one embodiment, a pre-filled syringe comprises baked on silicone. In another embodiment, a pre-filled syringe is free from silicone based lubricants. One of skill in the art also understands that small amounts of contaminating elements leaching into the formulation from the syringe barrel, syringe tip cap, plunger or stopper may also influence stability of the formulation. For example, it is understood that tungsten introduced during the manufacturing process may adversely affect formulation stability. In one embodiment, a pre-filled syringe may comprise tungsten at a level above 500 ppb. In another embodiment, a pre-filled syringe is a low tungsten syringe. In another embodiment, a pre-filled syringe may comprise tungsten at a level between about 500 ppb and about 10 ppb, between about 400 ppb and about 10 ppb, between about 300 ppb and about 10 ppb, between about 200 ppb and about 10 ppb, between about 100 ppb and about 10 ppb, between about 50 ppb and about 10 ppb, between about 25 ppb and about 10 ppb.

The present invention provides kits that can be used in the above methods. In one embodiment, a kit comprises a liquid formulation of the invention, in one or more containers. In another embodiment, a kit comprises a liquid formulation of the invention, in one or more containers, and one or more other prophylactic or therapeutic agents useful for the prevention, management or treatment of a disease or disorder. The disease or disorder may be associated with or characterized by aberrant expression and/or activity of an interferon alpha polypeptide, a disease or disorder associated with or characterized by aberrant expression and/or activity of the interferon alpha receptor or one or more subunits thereof, an autoimmune disease, an autoimmune disease, transplant rejection, graft versus host disease, or one or more symptoms thereof, in one or more other containers. In a specific embodiment, the antibody (including antibody fragments thereof) included in said liquid formulations is 13H5 or an antigen-binding fragment. In an alternative embodiment, the antibody (including antibody fragment thereof) included in said liquid formulations is not 13H5 or an antigen-binding fragment thereof. The kit may further comprise instructions for preventing, treating and/or managing a disorder (e.g., using the liquid formulations of the invention alone or in combination with another prophylactic or therapeutic agent), as well as side effects and dosage information for method of administration.

### 5.12. Articles of Manufacture

The present invention also encompasses a finished packaged and labeled pharmaceutical product. This article of manufacture includes the appropriate unit dosage form in an appropriate vessel or container such as a glass vial, pre-filled syringe or other container that is hermetically sealed. The unit dosage form is provided as a sterile particulate free solution comprising an anti-interferon alpha antibody that is suitable for parenteral administration.

In one embodiment, the unit dosage form is suitable for intravenous, intramuscular, intranasal, oral, topical or subcutaneous delivery. Thus, the invention encompasses sterile solutions suitable for each delivery route.

As with any pharmaceutical product, the packaging material and container are designed to protect the stability of the product during storage and shipment. Further, the products of the invention include instructions for use or other informational material that advise the physician, technician or patient on how to appropriately prevent or treat the disease or disorder in question. In other words, the article of manufacture includes instruction means indicating or suggesting a dosing regimen including, but not limited to, actual doses, monitoring procedures, and other monitoring information.

Specifically, the invention provides an article of manufacture comprising packaging material, such as a box, bottle, tube, vial, container, pre-filled syringe, sprayer, insufflator, intravenous (i.v.) bag, envelope and the like; and at least one unit dosage form of a pharmaceutical agent contained within said packaging material, wherein said pharmaceutical agent comprises a liquid formulation containing an antibody. The packaging material includes instruction means which indicate that said antibody can be used to prevent, treat and/or manage one or more symptoms associated with a disorder associated with aberrant expression and/or activity of, e.g., an interferon alpha polypeptide, a disorder associated with aberrant expression and/or activity of an interferon alpha receptor or one or more subunits thereof, an autoimmune disorder, an inflammatory disorder, a proliferative disorder, an infection, or one or more symptoms thereof by administering specific doses and using specific dosing regimens as described herein.

The invention also provides an article of manufacture comprising packaging material, such as a box, bottle, tube, vial, container, pre-filled syringe, sprayer, insufflator, intravenous (i.v.) bag, envelope and the like; and at least one unit dosage form of each pharmaceutical agent contained within said packaging material, wherein one pharmaceutical agent comprises a liquid formulation containing an antibody that specifically binds to an interferon alpha polypeptide and the other pharmaceutical agent comprises a prophylactic or therapeutic agent other than an antibody that specifically binds to an interferon alpha polypeptide, and wherein said packaging material includes instruction means which indicate that said agents can be used to prevent, treat and/or manage one or more symptoms associated with a disorder associated with aberrant expression and/or activity of an interferon alpha polypeptide, a disorder associated with aberrant expression and/or activity of an interferon alpha receptor or one or more subunits thereof, an autoimmune disorder, an inflammatory disorder, a proliferative disorder, an infection, or one or more symptoms thereof by administering specific doses and using specific dosing regimens as described herein.

The present invention provides that the adverse effects that may be reduced or avoided by the methods of the invention are indicated in informational material enclosed in an article of manufacture for use in preventing, treating and/or managing one or more symptoms associated with an autoimmune disorder, an inflammatory disorder or an infection. Adverse effects that may be reduced or avoided by the methods of the invention include, but are not limited to, vital sign abnormalities (fever, tachycardia, bardycardia, hypertension, hypotension), hematological events (anemia, lymphopenia, leukopenia, thrombocytopenia), headache, chills, dizziness, nausea, asthenia, back pain, chest pain (chest pressure), diarrhea, myalgia, pain, pruritus, psoriasis, rhinitis, sweating, injection site reaction, and vasodilatation.

Further, the information material enclosed in an article of manufacture described herein can indicate that foreign proteins may also result in allergic reactions, including anaphylaxis, or cytosine release syndrome. The information material should indicate that allergic reactions may exhibit only as mild pruritic rashes or they may be severe such as erythroderma, Stevens-Johnson syndrome, vasculitis, or anaphylaxis. The information material should also indicate that anaphylactic reactions (anaphylaxis) are serious and occasionally fatal hypersensitivity reactions. Allergic reactions including anaphylaxis may occur when any foreign protein is injected into the body. They may range from mild manifestations such as urticaria or rash to lethal systemic reactions. Anaphylactic reactions occur soon after exposure, usually within 10 minutes. Patients may experience paresthesia, hypotension, laryngeal edema, mental status changes, facial or pharyngeal angioedema, airway obstruction, bronchospasm, urticaria and pruritus, serum sickness, arthritis, allergic nephritis, glomerulonephritis, temporal arthritis, or eosinophilia.

### 5.13. Specific Embodiments

What is embodimented is:

1. A sterile, stable aqueous formulation comprising an antibody or fragment thereof that specifically binds human interferon alpha.

2. The formulation of embodiment 1, wherein said antibody or fragment thereof was not subjected to lyophilization.

3. The formulation of embodiment 1, wherein said antibody or a fragment thereof is from an immunoglobulin type selected from the group consisting of IgA, IgE, IgM, IgD, IgY and IgG.

4. The formulation of embodiment 1, wherein said antibody or a fragment thereof is of the IgG1, IgG2, IgG3, or IgG4 human isotype.

5. The formulation of embodiment 1, wherein said antibody or a fragment thereof is a murine antibody or a fragment thereof, a chimeric antibody or a fragment thereof, a humanized antibody or a fragment thereof, or human antibody or a fragment thereof.

6. The formulation of any one of embodiments 1 to 5, wherein said antibody or fragment thereof comprises a heavy chain variable sequence of SEQ ID NO: 1.

7. The formulation of any one of embodiments 1 to 5, wherein said antibody or fragment thereof comprises a light chain variable sequence of SEQ ID NO:2.

8. The formulation of any one of embodiments 1 to 5, wherein said antibody or fragment thereof comprises a heavy chain variable sequence of SEQ ID NO:1 and a light chain variable sequence of SEQ ID NO:2.

9. The formulation of any one of embodiments 1 to 5, wherein said antibody is the 13H5 anti-human interferon alpha antibody.

10. The formulation of any one of embodiments 1 to 9, wherein the concentration of said antibody or fragment thereof is at a concentration of at least 50 mg/ml, at least 60 mg/ml, at least 70 mg/ml, at least 80 mg/ml, at least 90 mg/ml, at least 100 mg/ml, at least 120 mg/ml, at least 150 mg/ml, at least 160 mg/ml, at least 180 mg/ml, at least 200 mg/ml, at least 250 mg/ml, or at least 300 mg/ml.

11. The formulation of any one of embodiments 1 to 9, wherein the concentration of said antibody or fragment thereof is at a concentration of at least 100 mg/ml.

12. The formulation of any one of embodiments 1 to 9, wherein the concentration of said antibody or fragment thereof is at a concentration of at least 125 mg/ml.

13. The formulation of any one of embodiments 1 to 9, wherein the concentration of said antibody or fragment thereof is at a concentration of at least 150 mg/ml.

14. The formulation of any one of embodiments 1 to 9, wherein the concentration of said antibody or fragment thereof is at a concentration of at least 175 mg/ml.

15. The formulation of any one of embodiments 1 to 9, wherein the concentration of said antibody or fragment thereof is at a concentration of at least 200 mg/ml

16. The formulation of any one of embodiments 1 to 9, wherein the concentration of said antibody or fragment thereof is at a concentration of between about 90 mg/ml and about 250 mg/ml.

17. The formulation of any one of embodiments 1 to 9, wherein the concentration of said antibody or fragment thereof is at a concentration of between about 110 mg/ml and about 250 mg/ml.

18. The formulation of any one of embodiments 1 to 17, wherein said formulation further comprises a buffering component.

19. The formulation of any one of embodiments 1 to 18, wherein said formulation further comprises an at least one excipient.

20. The formulation of embodiments 18 or 19, wherein said buffering component is selected from the group consisting of histidine, citrate, phosphate, glycine, and acetate.

21. The formulation of embodiments 18 or 19, wherein said buffering component is histidine.

22. The formulation of embodiment 21, wherein said histidine is at a concentration from about 1 nM to about 200 nM.

23. The formulation of embodiment 21, wherein said histidine is at a concentration from about 10 nM to about 50 nM.

24. The formulation of embodiment 21, wherein said histidine is at a concentration from about 20 nM to about 30 nM.

25. The formulation of embodiment 21, wherein said histidine is at a concentration of about 25 nM.

26. The formulation of any one of embodiments 18 or 19, wherein said buffering component is citrate.

27. The formulation of embodiment 26, wherein said citrate is at a concentration from about 1 nM to about 200 nM.

28. The formulation of embodiment 26, wherein said citrate is at a concentration from about 10 nM to about 50 nM.

29. The formulation of embodiment 26, wherein said citrate is at a concentration from about 20 nM to about 30 nM.

30. The formulation of embodiment 26, wherein said citrate is at a concentration of about 25 nM.

31. The formulation of embodiment 19 wherein said excipient is a saccharide.

32. The formulation of embodiment 31, wherein said saccharide is a disaccharide.

33. The formulation of embodiment 32, wherein said disaccharide is trehalose or sucrose.

34. The formulation of embodiment 32, wherein said disaccharide is trehalose.

35. The formulation of embodiment 34, wherein said trehalose is at a concentration from about 1% to about 40%.

36. The formulation of embodiment 34, wherein said trehalose is at a concentration from about 2% to about 20%.

37. The formulation of embodiment 34, wherein said trehalose is at a concentration from about 4% to about 15%.

38. The formulation of embodiment 34, wherein said trehalose is at a concentration of about 8%.

39. The formulation of embodiment 32, wherein said disaccharide is sucrose.

40. The formulation of embodiment 39, wherein said sucrose is at a concentration from about 1% to about 40%.

41. The formulation of embodiment 39, wherein said sucrose is at a concentration from about 2% to about 20%.

42. The formulation of embodiment 39, wherein said sucrose is at a concentration from about 2% to about 15%.

43. The formulation of embodiment 39, wherein said sucrose is at a concentration of about 5%.

44. The formulation of embodiment 19, wherein said excipient is a polyol.

45. The formulation of embodiment 44, wherein said polyol is mannitol.

46. The formulation of embodiment 45, wherein said mannitol is at a concentration from about 0.1% to about 10%.

47. The formulation of embodiment 45, wherein said mannitol is at a concentration from about 0.5% to about 5%.

48. The formulation of embodiment 45, wherein said mannitol is at a concentration of about 1.5%.

49. The formulation of embodiment 19, wherein said excipient is a salt.

50. The formulation of embodiment 49, wherein said salt is sodium chloride.

51. The formulation of embodiment 50, wherein said sodium chloride is at a concentration from about 50 mM to about 200 mM.

52. The formulation of embodiment 50, wherein said sodium chloride is at a concentration of about 125 mM.

53. The formulation of embodiment 19, wherein said excipient is a surfactant.

54. The formulation of embodiment 53, wherein said surfactant is a polysorbate.

55. The formulation of embodiment 54, wherein said polysorbate is polysorbate 20 or polysorbate 80.

56. The formulation of embodiment 54, wherein said polysorbate is polysorbate 80.

57. The formulation of embodiment 56, wherein said polysorbate 80 is at a concentration from about 0.001% to about 2%.

58. The formulation of embodiment 56, wherein said polysorbate 80 is at a concentration of about 0.02%.

59. The formulation of any one of embodiments 1 to 58, wherein said formulation has a pH of between about 5.5 and 6.5.

60. The formulation of any one of embodiments 1 to 58, wherein said formulation has a pH of about 6.0.

61. The formulation of any one of embodiments 1 to 60, wherein said formulation is isotonic.

62. The formulation of any one of embodiments 1 to 61, wherein said formulation is stable upon storage at about 40° C. for at least 4 weeks.

63. The formulation of any one of embodiments 1 to 61, wherein said formulation is stable upon storage at about 5° C. for at least 3 months.

64. The formulation of any one of embodiments 1 to 61, wherein said formulation is stable upon storage at about 5° C. for at least 12 months.

65. The formulation of any one of embodiments 1 to 61, wherein said antibody or fragment thereof retains at least 80% of binding ability to a human interferon alpha polypeptide compared to a reference antibody representing the antibody prior to the storage at about 40° C. for at least 4 weeks.

66. The formulation of any one of embodiments 1 to 61, wherein said antibody or fragment thereof retains at least 80% of binding ability to a human interferon alpha polypeptide compared to a reference antibody representing the antibody prior to the storage at about 5° C. for at least 3 months.

67. The formulation of any one of embodiments 1 to 61, wherein said antibody or fragment thereof retains at least 80% of binding ability to a human interferon alpha polypeptide compared to a reference antibody representing the antibody prior to the storage at about 5° C. for at least 12 months.

68. The formulation of any one of embodiments 1 to 61, wherein said antibody or fragment thereof retains at least 90% of binding ability to a human interferon alpha polypeptide compared to a reference antibody representing the antibody prior to the storage at about 40° C. for at least 4 weeks.

69. The formulation of any one of embodiments 1 to 61, wherein said antibody or fragment thereof retains at least 90% of binding ability to a human interferon alpha polypeptide compared to a reference antibody representing the antibody prior to the storage at about 5° C. for at least 3 months.

70. The formulation of any one of embodiments 1 to 61, wherein said antibody or fragment thereof retains at least 90% of binding ability to a human interferon alpha polypeptide compared to a reference antibody representing the antibody prior to the storage at about 5° C. for at least 12 months.

71. The formulation of any one of embodiments 1 to 61, wherein said antibody or fragment thereof retains at least 95% of binding ability to a human interferon alpha polypeptide compared to a reference antibody representing the antibody prior to the storage at about 40° C. for at least 4 weeks.

72. The formulation of any one of embodiments 1 to 61, wherein said antibody or fragment thereof retains at least 95% of binding ability to a human interferon alpha polypeptide compared to a reference antibody representing the antibody prior to the storage at about 5° C. for at least 3 months.

73. The formulation of any one of embodiments 1 to 61, wherein said antibody or fragment thereof retains at least 95% of binding ability to a human interferon alpha polypeptide compared to a reference antibody representing the antibody prior to the storage at about 5° C. for at least 12 months.

74. The formulation of any one of embodiments 1 to 61, wherein said antibody or fragment thereof is susceptible to aggregation, fragmentation, or deamidation.

75. The formulation of any one of embodiments 1 to 61, wherein less than 2% of said antibody or fragment thereof forms an aggregate upon storage at about 40° C. for at least 4 weeks as determined by as determined by HPSEC.

76. The formulation of any one of embodiments 1 to 61, wherein less than 2% of said antibody or fragment thereof forms an aggregate upon storage at about 5° C. for at least 3 months as determined by HPSEC.

77. The formulation of any one of embodiments 1 to 61, wherein less than 2% of said antibody or fragment thereof forms an aggregate upon storage at about 5° C. for at least 12 months as determined by HPSEC.

78. The formulation of any one of embodiments 1 to 61, wherein less than 5% of said antibody or fragment thereof is fragmented upon storage at about 40° C. for at least 4 weeks as determined by RP-HPLC.

79. The formulation of any one of embodiments 1 to 61, wherein less than 5′% of said antibody or fragment thereof is fragmented upon storage at about 5° C. for at least 3 months as determined by RP-HPLC.

80. The formulation of any one of embodiments 1 to 61, wherein less than 5% of said antibody or fragment thereof is fragmented upon storage at about 5° C. for at least 12 months as determined by RP-HPLC.

81. The formulation of any one of embodiments 1 to 61, wherein less than 60% of said antibody or fragment thereof is subject to deamidation upon storage at about 40° C. for at least 4 weeks as determined by IEC.

82. The formulation of any one of embodiments 1 to 61, wherein less than 30% of said antibody or fragment thereof is subject to deamidation upon storage at about 5° C. for at least 3 months as determined by IEC.

83. The formulation of any one of embodiments 1 to 61, wherein less than 60% of said antibody or fragment thereof is subject to deamidation upon storage at about 5° C. for at least 12 months as determined by IEC.

84. The formulation of any one of embodiments 1 to 61, wherein said formulation is clear and colorless upon storage at about 5° C. for at least 3 months as determined by visual inspection.

85. The formulation of any one of embodiments 1 to 61, wherein said formulation is clear and colorless upon storage at about 5° C. for at least 12 months as determined by visual inspection.

86. The formulation of any one of embodiments 1 to 85, wherein said formulation is an injectable formulation.

87. The formulation of embodiment 86, wherein said formulation is suitable for intravenous, subcutaneous, or intramuscular administration.

88. The formulation of embodiment 87, wherein said formulation is suitable for intravenous administration and the antibody or antibody fragment concentration is from about 20 mg/ml to about 40 mg/ml.

89. The formulation of embodiment 87, wherein said formulation is suitable for subcutaneous administration and the antibody or antibody fragment concentration is from about 70 mg/ml to about 250 mg/ml.

90. The formulation of any one of embodiments 1 to 85, wherein said formulation is suitable for aerosol administration.

91. A pharmaceutical unit dosage form suitable for parenteral administration to a human which comprises an antibody formulation of any one of embodiments 1 to 85 in a suitable container.

92. The pharmaceutical unit dosage form of embodiment 91, wherein the antibody formulation is administered intravenously, subcutaneously, or intramuscularly.

93. A pharmaceutical unit dosage form suitable for aerosol administration to a human which comprises an antibody formulation of any one of embodiments 1 to 85 in a suitable container.

94. The pharmaceutical unit dosage of embodiment 93, wherein the antibody formulation is administered intranasally.

95. A sealed container containing the formulation of any one of embodiments 1 to 90.

96. A kit comprising the formulation of any one of embodiments 1 to 90.

97. A method of preventing, managing, treating or ameliorating an inflammatory disease or disorder, an autoimmune disease or disorder, a proliferative disease, an infection, a disease or disorder associated with or characterized by aberrant expression and/or activity of an interferon alpha polypeptide, a disease or disorder associated with or characterized by aberrant expression and/or activity of the interferon alpha receptor or one or more subunits thereof, or one or more symptoms thereof, said method comprising administering to a subject in need thereof a prophylactically or therapeutically effective amount of an antibody formulation of any one of embodiments 1 to 90.

98. The method of embodiment 97, wherein the disease or disorder is systemic lupus erythematosus.

99. The method of embodiment 97, wherein the disease or disorder is selected from the group consisting of multiple sclerosis, inflammatory bowel disease, insulin dependent diabetes mellitus, psoriasis, autoimmune thyroiditis, rheumatoid arthritis, glomerulonephritis, idiopathic inflammatory myopathies (IIM), dermatomyositis (DM), polymyositis (PM), and inclusion body myositis (IBM).

100. The method of embodiment 97, wherein the disease or disorder is transplant rejection or graft versus host disease.

101. The method of embodiment 97, further comprising administering to said subject a prophylactically or therapeutically effective amount of a prophylactic or therapeutic agent other than an antibody or antibody fragment that specifically binds to an interferon alpha polypeptide.

102. The method of embodiment 101, wherein the prophylactic or therapeutic agent is an anti-inflammatory agent, immunomodulatory agent, anti-angiogenic agent, or anti-cancer agent.

103. A sterile, stable aqueous formulation comprising a 13H5 anti-human interferon alpha antibody, and further comprising histidine, sodium chloride, sucrose, trehalose or polysorbate 80.

104. The composition of embodiment 103, wherein said composition comprises a 13H5 anti-human interferon alpha antibody, histidine, trehalose and polysorbate 80.

105. The composition of embodiment 104, wherein said composition comprises between about 50 mg/ml and about 150 mg/ml of a 13H5 anti-human interferon alpha antibody, between about 1 mM and about 100 mM histidine, between about 1% and about 40% trehalose and between about 0.001% and about 5% polysorbate 80 and wherein the pH of said composition is between about 5 and about 7.

106. The composition of embodiment 104, wherein said composition comprises between about 80 mg/ml and about 120 mg/ml of a 13H5 anti-human interferon alpha antibody, between about 10 mM and about 50 mM histidine, between about 4% and about 20% trehalose and between about 0.005% and about 1% polysorbate 80 and wherein the pH of said composition is between about 5.5 and about 6.5.

107. The composition of embodiment 104, wherein said composition comprises about 100 mg/ml of a 13H5 anti-human interferon alpha antibody, about 25 mM histidine, about 8% trehalose and about 0.02% polysorbate 80 and wherein the pH of said composition is about 6.

108. The composition of embodiment 103, wherein said composition comprises a 13H5 anti-human interferon alpha antibody, histidine, sucrose and polysorbate 80.

109. The composition of embodiment 108, wherein said composition comprises about 100 mg/ml of a 13H5 anti-human interferon alpha antibody, about 25 mM histidine, about 5% sucrose and about 0.02% polysorbate 80 and wherein the pH of said composition is about 6.

110. The composition of embodiment 108, wherein said composition comprises about 125 mg/ml of a 13H5 anti-human interferon alpha antibody, about 25 mM histidine, about 5% sucrose and about 0.02% polysorbate 80 and wherein the pH of said composition is about 6.

111. The composition of embodiment 108, wherein said composition comprises about 150 mg/ml of a 13H5 anti-human interferon alpha antibody, about 25 mM histidine, about 5% sucrose and about 0.02% polysorbate 80 and wherein the pH of said composition is about 6.

112. The composition of embodiment 108, wherein said composition comprises about 175 mg/ml of a 13H5 anti-human interferon alpha antibody, about 25 mM histidine, about 5% sucrose and about 0.02% polysorbate 80 and wherein the pH of said composition is about 6.

113. The composition of embodiment 108, wherein said composition comprises about 200 mg/ml of a 13H5 anti-human interferon alpha antibody, about 25 mM histidine, about 5% sucrose and about 0.02% polysorbate 80 and wherein the pH of said composition is about 6.

114. The composition of any one of embodiments 104 to 113, wherein said composition is isotonic.

115. The composition of any one of embodiments 104 to 113, wherein said formulation is stable upon storage at about 40° C. for at least 4 weeks.

116. The composition of any one of embodiments 104 to 113, wherein said formulation is stable upon storage at about 5° C. for at least 3 months.

117. The composition of any one of embodiments 104 to 113, wherein said formulation is stable upon storage at about 5° C. for at least 12 months.

118. The composition of any one of embodiments 104 to 113, wherein said antibody or fragment thereof retains at least 80% of binding ability to a human interferon alpha polypeptide compared to a reference antibody representing the antibody prior to the storage at about 40° C. for at least 4 weeks.

119. The composition of any one of embodiments 104 to 113, wherein said antibody or fragment thereof retains at least 80% of binding ability to a human interferon alpha polypeptide compared to a reference antibody representing the antibody prior to the storage at about 5° C. for at least 3 months.

120. The composition of any one of embodiments 104 to 113, wherein said antibody or fragment thereof retains at least 80% of binding ability to a human interferon alpha polypeptide compared to a reference antibody representing the antibody prior to the storage at about 5° C. for at least 12 months.

121. The composition of any one of embodiments 104 to 113, wherein said antibody or fragment thereof retains at least 90% of binding ability to a human interferon alpha polypeptide compared to a reference antibody representing the antibody prior to the storage at about 40° C. for at least 4 weeks.

122. The composition of any one of embodiments 104 to 113, wherein said antibody or fragment thereof retains at least 90% of binding ability to a human interferon alpha polypeptide compared to a reference antibody representing the antibody prior to the storage at about 5° C. for at least 3 months.

123. The composition of any one of embodiments 104 to 113, wherein said antibody or fragment thereof retains at least 90% of binding ability to a human interferon alpha polypeptide compared to a reference antibody representing the antibody prior to the storage at about 5° C. for at least 12 months.

124. The composition of any one of embodiments 104 to 113, wherein said antibody or fragment thereof retains at least 95% of binding ability to a human interferon alpha polypeptide compared to a reference antibody representing the antibody prior to the storage at about 40° C. for at least 4 weeks.

125. The composition of any one of embodiments 104 to 113, wherein said antibody or fragment thereof retains at least 95% of binding ability to a human interferon alpha polypeptide compared to a reference antibody representing the antibody prior to the storage at about 5° C. for at least 3 months.

126. The composition of any one of embodiments 104 to 113, wherein said antibody or fragment thereof retains at least 95% of binding ability to a human interferon alpha polypeptide compared to a reference antibody representing the antibody prior to the storage at about 5° C. for at least 12 months.

127. The composition of any one of embodiments 104 to 113, wherein said antibody or fragment thereof is susceptible to aggregation, fragmentation, or deamidation.

128. The composition of any one of embodiments 104 to 113, wherein less than 2% of said antibody or fragment thereof forms an aggregate upon storage at about 40° C. for at least 4 weeks as determined by as determined by HPSEC.

129. The composition of any one of embodiments 104 to 113, wherein less than 2% of said antibody or fragment thereof forms an aggregate upon storage at about 5° C. for at least 3 months as determined by HPSEC.

130. The composition of any one of embodiments 104 to 113, wherein less than 2% of said antibody or fragment thereof forms an aggregate upon storage at about 5° C. for at least 12 months as determined by HPSEC.

131. The composition of any one of embodiments 104 to 113, wherein less than 5% of said antibody or fragment thereof is fragmented upon storage at about 40° C. for at least 4 weeks as determined by RP-HPLC.

132. The composition of any one of embodiments 104 to 113, wherein less than 5% of said antibody or fragment thereof is fragmented upon storage at about 5° C. for at least 3 months as determined by RP-HPLC.

133. The composition of any one of embodiments 104 to 113, wherein less than 5% of said antibody or fragment thereof is fragmented upon storage at about 5° C. for at least 12 months as determined by RP-HPLC.

134. The composition of any one of embodiments 104 to 113, wherein less than 60% of said antibody or fragment thereof is subject to deamidation upon storage at about 40° C. for at least 4 weeks as determined by IEC.

135. The composition of any one of embodiments 104 to 113, wherein less than 30% of said antibody or fragment thereof is subject to deamidation upon storage at about 5° C. for at least 3 months as determined by IEC.

136. The composition of any one of embodiments 104 to 113, wherein less than 60% of said antibody or fragment thereof is subject to deamidation upon storage at about 5° C. for at least 12 months as determined by IEC.

137. The composition of any one of embodiments 104 to 113, wherein said formulation is clear and colorless upon storage at about 5° C. for at least 3 months as determined by visual inspection.

138. The composition of any one of embodiments 104 to 113, wherein said formulation is clear and colorless upon storage at about 5° C. for at least 12 months as determined by visual inspection.

139. The composition of any one of embodiments 104 to 113, wherein said formulation is an injectable formulation.

140. The composition of embodiment 139, wherein said formulation is suitable for intravenous, subcutaneous, or intramuscular administration.

141. The formulation of embodiment 140, wherein said formulation is suitable for intravenous administration.

142. The formulation of embodiment 140, wherein said formulation is suitable for subcutaneous administration.

143. The composition of any one of embodiments 104 to 113, wherein said formulation is suitable for aerosol administration.

144. A process for the preparation of a composition according to any one of embodiments 104 to 113 comprising:

a) concentrating a 13H5 antibody solution to between about 10 mg/ml and about 50 mg/ml;

b) diafiltering said concentrated 13H5 antibody with a solution comprising histidine.

145. The process of embodiment 144 further comprising:

(c) concentrating said 13H5 antibody diafilterd with a solution comprising histidine to between about 50 mg/ml and 250 mg/ml;

(d) admixing said concentrated 13H5 solution with at least one solution comprising at least one excipient.

146. A method for stabilizing a 13H5 antibody comprising combining said antibody with histidine-HCl, trehalose and polysorbate 80 at a pH of about 6.

147. The method of embodiment 146, wherein said 13H5 antibody concentration is between about 80 mg/ml and about 120 mg/ml.

148. A method for stabilizing a 13H5 antibody comprising combining said antibody with histidine-HCl, sucrose and polysorbate 80 at a pH of about 6.

149. The method of embodiment 148, wherein said 13H5 antibody concentration is between about 90 mg/ml and about 210 mg/ml.

150. A pharmaceutical unit dosage form suitable for parenteral administration to a human which comprises an antibody formulation of any one of embodiments 104 to 143 in a suitable container.

151. The pharmaceutical unit dosage form of embodiment 150, wherein the antibody formulation is administered intravenously, subcutaneously, or intramuscularly.

152. A pharmaceutical unit dosage form suitable for aerosol administration to a human which comprises an antibody formulation of any one of embodiments 104 to 143 in a suitable container.

153. The pharmaceutical unit dosage of embodiment 152, wherein the antibody formulation is administered intranasally.

154. A sealed container containing the formulation of any one of embodiments 104 to 143.

155. A kit comprising the formulation of any one of embodiments 104 to 143.

156. A method of preventing, managing, treating or ameliorating an inflammatory disease or disorder, an autoimmune disease or disorder, a proliferative disease, an infection, a disease or disorder associated with or characterized by aberrant expression and/or activity of an interferon alpha polypeptide, a disease or disorder associated with or characterized by aberrant expression and/or activity of the interferon alpha receptor or one or more subunits thereof, or one or more symptoms thereof, said method comprising administering to a subject in need thereof a prophylactically or therapeutically effective amount of an antibody formulation of any one of embodiments 104 to 143.

157. The method of embodiment 156, wherein the disease or disorder is systemic lupus erythematosus.

158. The method of embodiment 156, wherein the disease or disorder is selected from the group consisting of multiple sclerosis, inflammatory bowel disease, insulin dependent diabetes mellitus, psoriasis, autoimmune thyroiditis, rheumatoid arthritis, glomerulonephritis, idiopathic inflammatory myopathies (IIM), dermatomyositis (DM), polymyositis (PM), and inclusion body myositis (IBM).

159. The method of embodiment 156, wherein the disease or disorder is transplant rejection or graft versus host disease.

160. The method of embodiment 156, further comprising administering to said subject a prophylactically or therapeutically effective amount of a prophylactic or therapeutic agent other than an antibody or antibody fragment that specifically binds to an interferon alpha polypeptide.

161. The method of embodiment 160, wherein the prophylactic or therapeutic agent is an anti-inflammatory agent, immunomodulatory agent, anti-angiogenic agent, or anti-cancer agent.

162. The formulation of any one of embodiments 1 to 90 or 103 to 143, wherein said formulation is a pharmaceutically acceptable formulation.

163. The embodiment of any one of embodiments 86, 87, 89, or 162, wherein the embodiment is in a pre-filled syringe.

164. The embodiment of embodiment 163, wherein the pre-filled syringe comprises a needle.

165. The embodiment of embodiment 163, wherein the pre-filled syringe is a plastic syringe or a glass syringe.

166. The embodiment of embodiment 163, wherein the pre-filled syringe is a plastic syringe.

167. The embodiment of embodiment 165, wherein the pre-filled syringe is a glass syringe.

168. The embodiment of the embodiment 163, wherein the pre-filled syringe is made of materials that are substantially free from tungsten.

169. The embodiment of embodiment 163, wherein the pre-filled syringe is substantially free from silicone.

170. The embodiment of embodiment 169, wherein the pre-filled syringe does not comprise a silicone based lubricant.

171. The embodiment of embodiment 163, wherein the pre-filled syringe comprises a plunger having a fluoropolymer resin disk.

172. The pharmaceutical unit dosage of any one of embodiments 91, 92, 150 or 151, wherein the suitable contained is a pre-filled syringe.

173. The pharmaceutical unit dosage of embodiment 172, wherein the pre-filled syringe comprises a needle.

174. The pharmaceutical unit dosage of embodiment 172, wherein the pre-filled syringe is a plastic syringe or a glass syringe.

175. The pharmaceutical unit dosage of embodiment 174, wherein the pre-filled syringe is a plastic syringe.

176. The pharmaceutical unit dosage of embodiment 174, wherein the pre-filled syringe is a glass syringe.

177. The pharmaceutical unit dosage of the embodiment 172, wherein the pre-filled syringe is made of materials that are substantially free from tungsten.

178. The pharmaceutical unit dosage of embodiment 172, wherein the pre-filled syringe is substantially free from silicone.

179. The pharmaceutical unit dosage of embodiment 178, wherein the pre-filled syringe does not comprise a silicone based lubricant.

180. The pharmaceutical unit dosage of embodiment 172, wherein the pre-filled syringe comprises a plunger having a fluoropolymer resin disk.

181. The sealed container of any one of embodiments 95 or 154, wherein the sealed container is in a pre-filled syringe.

182. The sealed container of embodiment 181, wherein the pre-filled syringe comprises a needle.

183. The sealed container of embodiment 181, wherein the pre-filled syringe is a plastic syringe or a glass syringe.

184. The sealed container of embodiment 183, wherein the pre-filled syringe is a plastic syringe.

185. The sealed container of embodiment 183, wherein the pre-filled syringe is a glass syringe.

186. The sealed container of the embodiment 181, wherein the pre-filled syringe is made of materials that are substantially free from tungsten.

187. The scaled container of embodiment 181, wherein the pre-filled syringe is substantially free from silicone.

188. The sealed container of embodiment 187, wherein the pre-filled syringe does not comprise a silicone based lubricant.

189. The sealed container of embodiment 181, wherein the pre-filled syringe comprises a plunger, wherein the plunger comprises a fluoropolymer resin disk.

190. The kit of any one of embodiments 96 or 155, wherein the kit comprises a pre-filled syringe.

191. The kit of embodiment 190, wherein the pre-filled syringe comprises a needle.

192. The kit of embodiment 190, wherein the pre-filled syringe is a plastic syringe or a glass syringe.

193. The kit of embodiment 192, wherein the pre-filled syringe is a plastic syringe.

194. The kit of embodiment 192, wherein the pre-filled syringe is a glass syringe.

195. The kit of the embodiment 190, wherein the pre-filled syringe is made of materials that are substantially free from tungsten.

196. The kit of embodiment 190, wherein the pre-filled syringe is substantially free from silicone.

197. The kit of embodiment 196, wherein the pre-filled syringe does not comprise a silicone based lubricant.

198. The kit of embodiment 190, wherein the pre-filled syringe comprises a plunger, wherein the plunger comprises a fluoropolymer resin disk.

199. The composition of any one of embodiments 139, 140 or 142, wherein the embodiment is in a pre-filled syringe.

200. The composition of embodiment 199, wherein the pre-filled syringe comprises a needle.

201. The composition of embodiment 199, wherein the pre-filled syringe is a plastic syringe or a glass syringe.

202. The composition of embodiment 201, wherein the pre-filled syringe is a plastic syringe.

203. The composition of embodiment 201, wherein the pre-filled syringe is a glass syringe.

204. The composition of the embodiment 199, wherein the pre-filled syringe is made of materials that are substantially free from tungsten.

205. The composition of embodiment 199, wherein the pre-filled syringe is substantially free from silicone.

206. The composition of embodiment 205, wherein the pre-filled syringe does not comprise a silicone based lubricant.

207. The composition of embodiment 199, wherein the pre-filled syringe comprises a plunger, wherein the plunger comprises a fluoropolymer resin disk.

208. A pre-filled syringe containing a sterile, stable aqueous embodiment comprising a 13H5 anti-human interferon alpha antibody, and further comprising histidine, sodium chloride, sucrose, trehalose or polysorbate 80.

209. The pre-filled syringe of embodiment 208, wherein the pre-filled syringe comprises a needle.

210. The pre-filled syringe of embodiment 208, wherein the pre-filled is sealed.

211. The pre-filled syringe of embodiment 208, wherein the pre-filled syringe is a plastic syringe or a glass syringe.

212. The pre-filled syringe of embodiment 211, wherein the pre-filled syringe is a plastic syringe.

213. The pre-filled syringe of embodiment 211, wherein the pre-filled syringe is a glass syringe.

214. The pre-filled syringe of embodiment 208, wherein the pre-filled syringe is made of materials that are substantially free from tungsten.

215. The pre-filled syringe of embodiment 208, wherein the pre-filled syringe is substantially free from silicone.

216. The pre-filled syringe of embodiment 209, wherein the pre-filled syringe does not comprise a silicone based lubricant.

217. The pre-filled syringe of embodiment 208, wherein the pre-filled syringe comprises a plunger having a fluoropolymer resin disk.

218. The pre-filled syringe of embodiment 208, wherein the syringe is a plastic syringe substantially free from silicone and tungsten comprising a plunger having a fluoropolymer resin disk.

219. The pre-filled syringe of any one of embodiments 208-218, wherein said embodiment comprises a 13H5 anti-human interferon alpha antibody, histidine, trehalose and polysorbate 80.

220. The pre-filled syringe of embodiment 219, wherein said embodiment comprises between about 50 mg/ml and about 150 mg/ml of a 13H5 anti-human interferon alpha antibody, between about 1 mM and about 100 mM histidine, between about 1% and about 40% trehalose and between about 0.001% and about 5% polysorbate 80 and wherein the pH of said embodiment is between about 5 and about 7.

221. The pre-filled syringe of embodiment 219, wherein said embodiment comprises between about 80 mg/ml and about 120 mg/ml of a 13H5 anti-human interferon alpha antibody, between about 10 mM and about 50 mM histidine, between about 4% and about 20% trehalose and between about 0.005% and about 1% polysorbate 80 and wherein the pH of said embodiment is between about 5.5 and about 6.5.

222. The pre-filled syringe of embodiment 219, wherein said embodiment comprises about 100 mg/ml of a 13H5 anti-human interferon alpha antibody, about 25 mM histidine, about 8% trehalose and between about 0.005% and about 1% polysorbate 80 and wherein the pH of said embodiment is about 6.

223. The pre-filled syringe of embodiment 219, wherein said embodiment comprises about 100 mg/ml of a 13H5 anti-human interferon alpha antibody, about 25 mM histidine, about 8% trehalose and about 0.02% polysorbate 80 and wherein the pH of said embodiment is about 6.

224. The pre-filled syringe of any one of embodiments 219 to 223, wherein said embodiment is isotonic.

225. The pre-filled syringe of any one of embodiments 219 to 223, wherein said embodiment is stable upon storage at about 40° C. for at least 4 weeks.

226. The pre-filled syringe of any one of embodiments 219 to 223, wherein said embodiment is stable upon storage at about 5° C. for at least 3 months.

227. The pre-filled syringe of any one of embodiments 219 to 223, wherein said embodiment is stable upon storage at about 5° C. for at least 12 months.

228. The pre-filled syringe of any one of embodiments 219 to 223, wherein said antibody or fragment thereof retains at least 80% of binding ability to a human interferon alpha polypeptide compared to a reference antibody representing the antibody prior to the storage at about 40° C. for at least 4 weeks.

229. The pre-filled syringe of any one of embodiments 219 to 223, wherein said antibody or fragment thereof retains at least 80% of binding ability to a human interferon alpha polypeptide compared to a reference antibody representing the antibody prior to the storage at about 5° C. for at least 3 months.

230. The pre-filled syringe of any one of embodiments 219 to 223, wherein said antibody or fragment thereof retains at least 80% of binding ability to a human interferon alpha polypeptide compared to a reference antibody representing the antibody prior to the storage at about 5° C. for at least 12 months.

231. The pre-filled syringe of any one of embodiments 219 to 223, wherein said antibody or fragment thereof retains at least 90% of binding ability to a human interferon alpha polypeptide compared to a reference antibody representing the antibody prior to the storage at about 40° C. for at least 4 weeks.

232. The pre-filled syringe of any one of embodiments 219 to 223, wherein said antibody or fragment thereof retains at least 90% of binding ability to a human interferon alpha polypeptide compared to a reference antibody representing the antibody prior to the storage at about 5° C. for at least 3 months.

233. The pre-filled syringe of any one of embodiments 219 to 223, wherein said antibody or fragment thereof retains at least 90% of binding ability to a human interferon alpha polypeptide compared to a reference antibody representing the antibody prior to the storage at about 5° C. for at least 12 months.

234. The pre-filled syringe of any one of embodiments 219 to 223, wherein said antibody or fragment thereof retains at least 95% of binding ability to a human interferon alpha polypeptide compared to a reference antibody representing the antibody prior to the storage at about 40° C. for at least 4 weeks.

235. The pre-filled syringe of any one of embodiments 219 to 223, wherein said antibody or fragment thereof retains at least 95% of binding ability to a human interferon alpha polypeptide compared to a reference antibody representing the antibody prior to the storage at about 5° C. for at least 3 months.

236. The pre-filled syringe of any one of embodiments 219 to 223, wherein said antibody or fragment thereof retains at least 95% of binding ability to a human interferon alpha polypeptide compared to a reference antibody representing the antibody prior to the storage at about 5° C. for at least 12 months.

237. The pre-filled syringe of any one of embodiments 219 to 223, wherein said antibody or fragment thereof is susceptible to aggregation, fragmentation, or deamidation.

238. The pre-filled syringe of any one of embodiments 219 to 223, wherein less than 2% of said antibody or fragment thereof forms an aggregate upon storage at about 40° C. for at least 4 weeks as determined by as determined by HPSEC.

239. The pre-filled syringe of any one of embodiments 219 to 223, wherein less than 2% of said antibody or fragment thereof forms an aggregate upon storage at about 5° C. for at least 3 months as determined by HPSEC.

240. The pre-filled syringe of any one of embodiments 219 to 223, wherein less than 2% of said antibody or fragment thereof forms an aggregate upon storage at about 5° C. for at least 12 months as determined by HPSEC.

241. The pre-filled syringe of any one of embodiments 219 to 223, wherein less than 5% of said antibody or fragment thereof is fragmented upon storage at about 40° C. for at least 4 weeks as determined by RP-HPLC.

242. The pre-filled syringe of any one of embodiments 219 to 223, wherein less than 5% of said antibody or fragment thereof is fragmented upon storage at about 5° C. for at least 3 months as determined by RP-HPLC.

243. The pre-filled syringe of any one of embodiments 219 to 223, wherein less than 5% of said antibody or fragment thereof is fragmented upon storage at about 5° C. for at least 12 months as determined by RP-HPLC.

244. The pre-filled syringe of any one of embodiments 219 to 223, wherein less than 60% of said antibody or fragment thereof is subject to deamidation upon storage at about 40° C. for at least 4 weeks as determined by IEC.

245. The pre-filled syringe of any one of embodiments 219 to 223, wherein less than 30% of said antibody or fragment thereof is subject to deamidation upon storage at about 5° C. for at least 3 months as determined by IEC.

246. The pre-filled syringe of any one of embodiments 219 to 223, wherein less than 60% of said antibody or fragment thereof is subject to dearnidation upon storage at about 5° C. for at least 12 months as determined by IEC.

247. The pre-filled syringe of any one of embodiments 219 to 223, wherein said embodiment is clear and colorless upon storage at about 5° C. for at least 3 months as determined by visual inspection.

248. The pre-filled syringe of any one of embodiments 219 to 223, wherein said embodiment is clear and colorless upon storage at about 5° C. for at least 12 months as determined by visual inspection.

249. The pre-filled syringe of any one of embodiments 219 to 223, wherein said embodiment is substantially free from particulates upon storage at about 5° C. for at least 3 months as determined by visual inspection.

250. The pre-filled syringe of any one of embodiments 219 to 223, wherein said embodiment is substantially free from particulates upon storage at about 5° C. for at least 12 months as determined by visual inspection.

251. The pre-filled syringe of any one of embodiments 219 to 223, wherein said embodiment is an injectable embodiment.

252. The pre-filled syringe of embodiment 251, wherein said embodiment is suitable for subcutaneous or intramuscular.

253. The pre-filled syringe of embodiment 252, wherein said embodiment is suitable for subcutaneous administration.

254. The prefilled syringe of any one of embodiments 219 to 253, wherein said embodiment is a pharmaceutically acceptable formulation.

255. A kit comprising the pre-filled syringe of any one of embodiments 219 to 254.

Those skilled in the art will recognize, or be able to ascertain using no more than routine experimentation, many equivalents to the specific embodiments of the invention described herein. Such equivalents are intended to be encompassed by the following claims.

All publications, patents and patent applications mentioned in this specification are herein incorporated by reference into the specification to the same extent as if each individual publication, patent or patent application was specifically and individually indicated to be incorporated herein by reference.

Citation or discussion of a reference herein shall not be construed as an admission that such is prior art to the present invention.

### 6. EXAMPLES

The invention is now described with reference to the following examples. These examples are provided for the purpose of illustration only and the invention should in no way be construed as being limited to these examples but rather should be construed to encompass any and all variations which become evident as a result of the teachings provided herein.

### 6.1. Example 1

### Formulation development for 13H5

The following section describes the characterization of various formulations comprising an anti-human interferon alpha antibody. Experimental results presented here were generated using the 13H5 antibody unless stated otherwise. 13H5 is a human IgG1 monoclonal antibody produced by recombinant DNA technology that that binds to IFN alpha and inhibits the biological activity of multiple IFN alpha subtypes, but does not substantially inhibit the biological activity of IFN alpha subtype 21, or of IFN beta or IFN omega. The 13H5 antibody used for the examples described herein comprises a heavy chain having the amino acid sequence of SEQ ID NO:1 and a light chain having the amino acid sequence of SEQ ID NO:6.

### 6.1.1. Experimental Methods

Purified 13H5 antibody is generated following standard industrial scale protocols. Details of cell culture condition and antibody purification are described in the co-pending U.S. Provisional Patent Application 60/909,232, filed on Mar. 30, 2007. The purified antibody is eluted from the final column in 10 mM sodium acetate (pH 5.2) at an approximate concentration of 2.5 mg/ml. Protein concentration is estimated from optical density measurement at 280 nm.

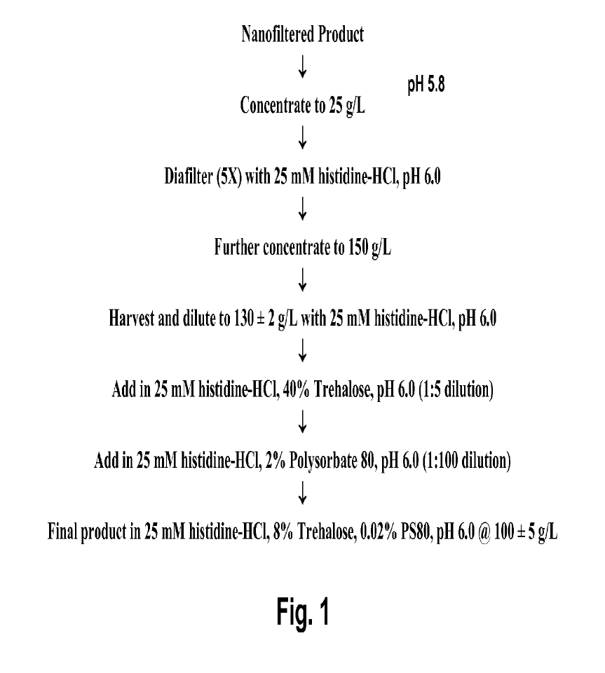

Purified 13H5 antibody is nanofiltered using a Planova 20N filter to remove particulate matter. 13H5 formulations are prepared using Tangential Flow Filtration (TFF). The nanofiltered 13H5 antibody is concentrated to approximately 25 mg/ml on a Millipore Labscale TFF device. The antibody is then 5× diafiltered into the appropriate buffer (e.g., 25 mM histidine-HCL (pH 6.0)). Once the buffer exchange is complete, the antibody is concentrated to approximately 150 mg/ml. Excipients are introduced by spiking the concentrated antibody preparation with the appropriate concentrated stock solutions. For example, a final concentration of 8% Trehalose is achieved by adding 25 ml of 25 mM histidine-HCl, 40% Trehalose (pH 6.0) to every 100 ml of concentrated antibody preparation. Multiple excipients may be introduced in consecutive steps. For example, a final concentration of 0.02% Polysorbate 80 is introduced after the addition of Trehalose by diluting 100 fold a 25 mM histidine-HCl, 8% Trehalose, 2% Polysorbate 80 (pH 6.0) stock solution with the Trehalose containing antibody preparation. The final concentration of 13H5 is adjusted to 100±5 mg/ml with the final formulation buffer (e.g., 25 mM histidine-HCl, 8% Trehalose, 0.01% Polysorbate 80 (pH 6.0)). A flow chart for the preparation of 13H5 formulations is presented in FIG. 1.

Details of various formulations tested for 13H5 are described in Table 1. Single dose aliquots of each formulation were stored for extended periods of time (e.g., 1 months, 2 months, 3 months etc.) before being subjected to analysis.

TABLE 1
13H5 formulations tested.

| | | | Conc. |
|---|---|---|---|
| Formulations | | pH | (mg/ml) |
| A | 25 mM Histidine, 125 mM Sodium Chloride, | 5.5 | 100 |
| | 0.02% Polysorbate 80 | | |
| B | 25 mM Histidine, 125 mM Sodium Chloride, | 6 | 50, 100 |
| | 0.02% Polysorbate 80 | | |
| C | 25 mM Histidine, 125 mM Sodium Chloride, | 6.5 | 100 |
| | 0.02% Polysorbate 80 | | |
| D | 25 mM Histidine, 125 mM Sodium Chloride, | 6 | 50, 100 |
| | 1.5% Trehalose, 0.02% Polysorbate 80 | | |
| E | 25 mM Histidine, 8% Trehalose, 0.02% | 6 | 50, 100 |
| | Polysorbate 80 | | |
| F | 20 mM Sodium Citrate, 1.5% Mannitol, 100 | 6 | 100 |
| | mM Sodium Chloride, 0.02% Polysorbate 80 | | |

The stability of each formulation was tested by analyzing the physical properties of single dose aliquots stored for extended periods of time. Some aliquots were stored under temperatures recommended for clinical storage (5° C.). Other aliquots were stored under elevated temperature (40° C.) to simulate the effects of very long term storage.

Additional storage conditions that may affect stability of a formulation include, but are not limited to, light intensity, light wavelength, humidity, vial composition, and stopper composition. The effect of these parameters on formulation stability may also be determined using the methods described herein.

Size exclusion chromatography was utilized to measure the amount of antibody aggregates in the formulation. SEC was performed using the Agilent 1100 Series High Performance Liquid Chromatography (HPLC) system. Samples were diluted to 10 mg/ml. 25 μl diluted sample containing 250 ug protein was injected onto a TSK-Gel 3000 column (size 7.8 mm×30.0 cm.; Tosoh Biosciences Corporation). Protein elution profile was determined by following the eluate's optical density at 280 nm. Data analysis was performed using ChemStation (Agilent) auto integration parameters. The percent of protein in aggregate form in various formulations is plotted in FIGS. 1-9

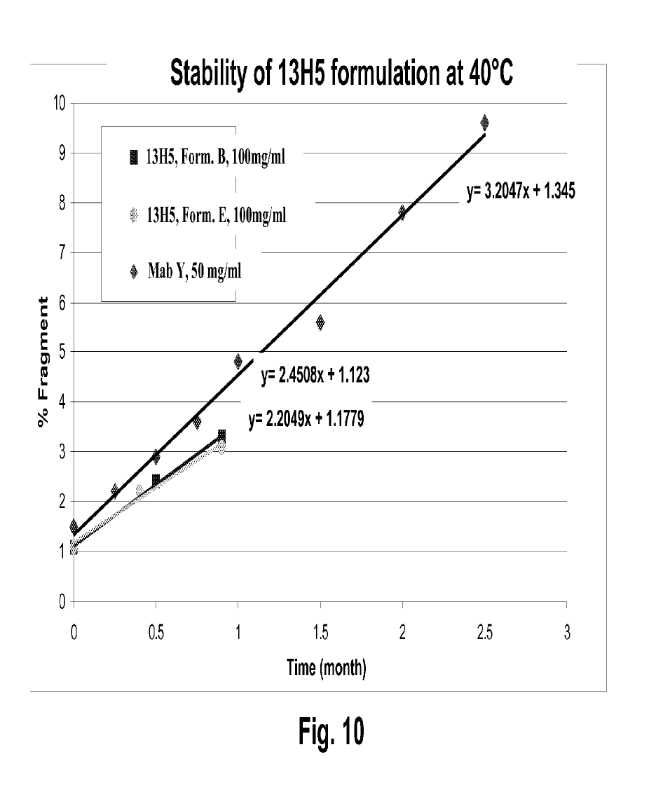

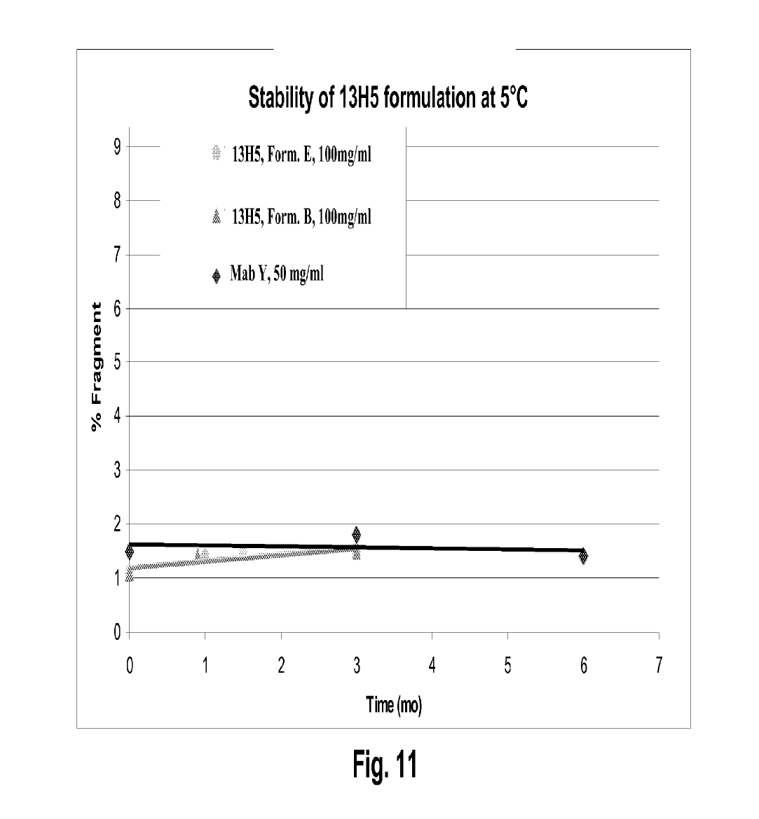

Reversed Phase High Performance Liquid Chromatography (RP-HPLC) was used to determine the amount of antibody fragments in the formulation. RP-HPLC was performed using the Agilent 1100 Series High Performance Liquid Chromatography (HPLC) system. Samples were analysed on a PLRP-S (8 um, 4000 A, 2.0×150 mm) column from Michrom Bioresources. Protein elution profile was determined by following the eluate's optical density at 280 nm. Data analysis was performed using ChemStation (Agilent) auto integration parameters. The percent of fragmented antibody in various formulations is plotted in FIGS. 10 and 11.

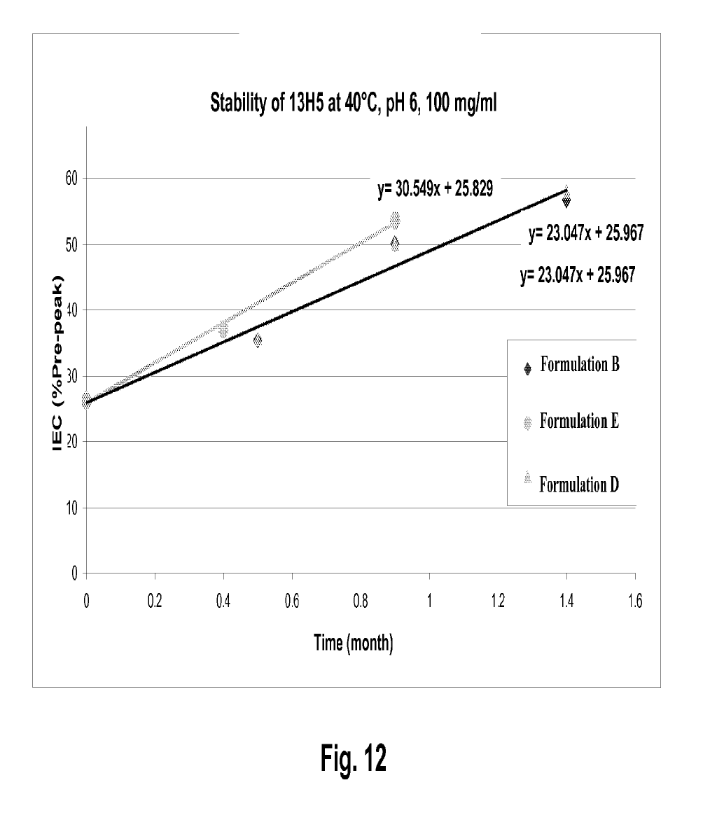

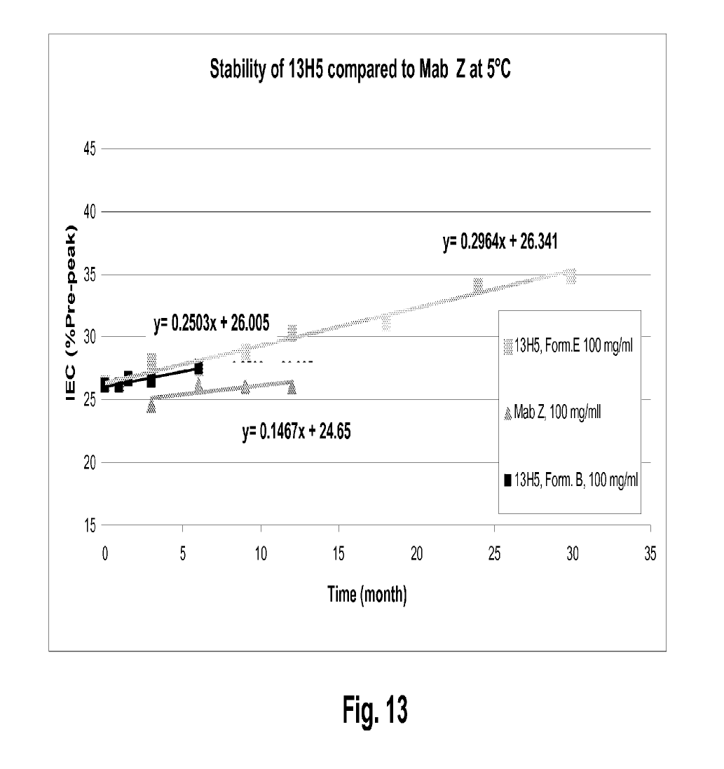

Ion exchange chromatography (IEC) was employed to measure the C-terminal Lysine and charge isoform heterogeneity of 13H5 in various formulations. Agilent 1100 Series High Performance Liquid Chromatography (HPLC) systems were used for this analysis. Samples were analysed o a Propac WCX-10G (4×250 mm) Analytical Column (Dionex). Data analysis was performed using the ChemStation (Agilent) auto integration parameters. The percent of pre-peak charge variant protein that elutes before the main antibody peak is plotted in FIGS. 12 and 13.

Visual inspection: color, clarity, and amount of particulates in a given formulation are determined by inspecting the sample with a naked eye.

### 6.1.2. Results

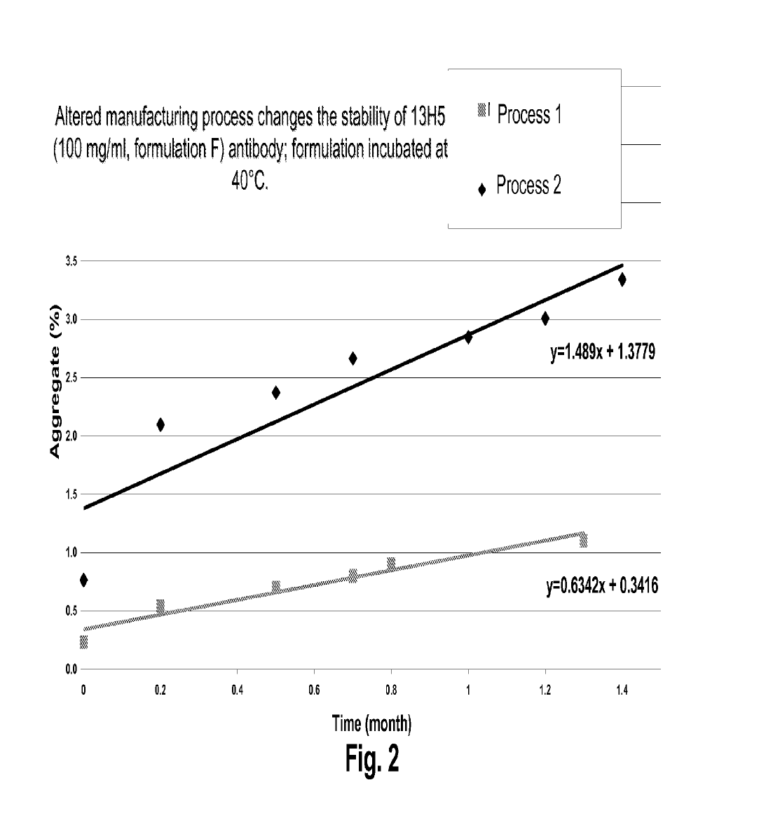

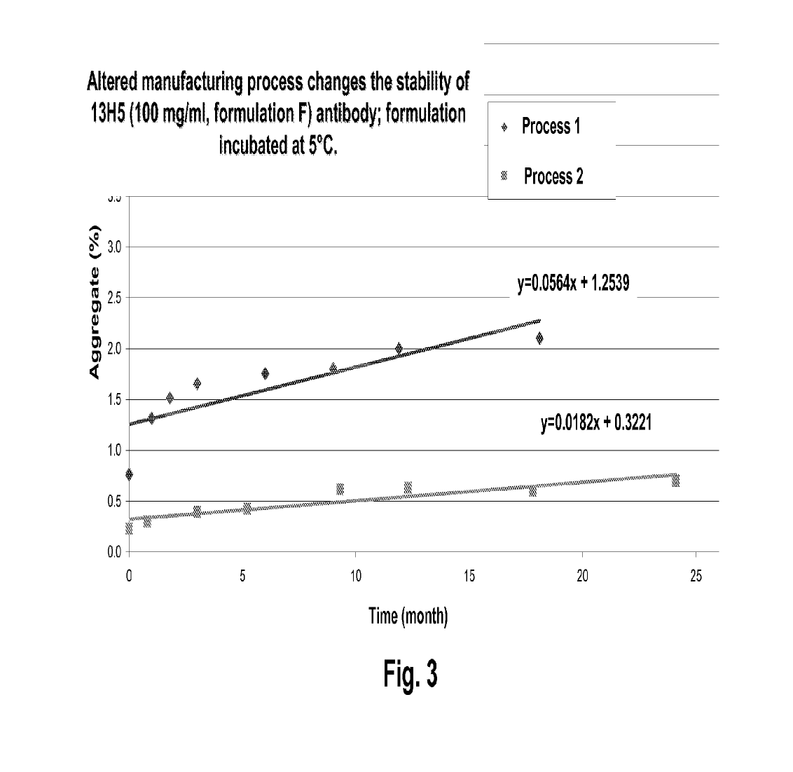

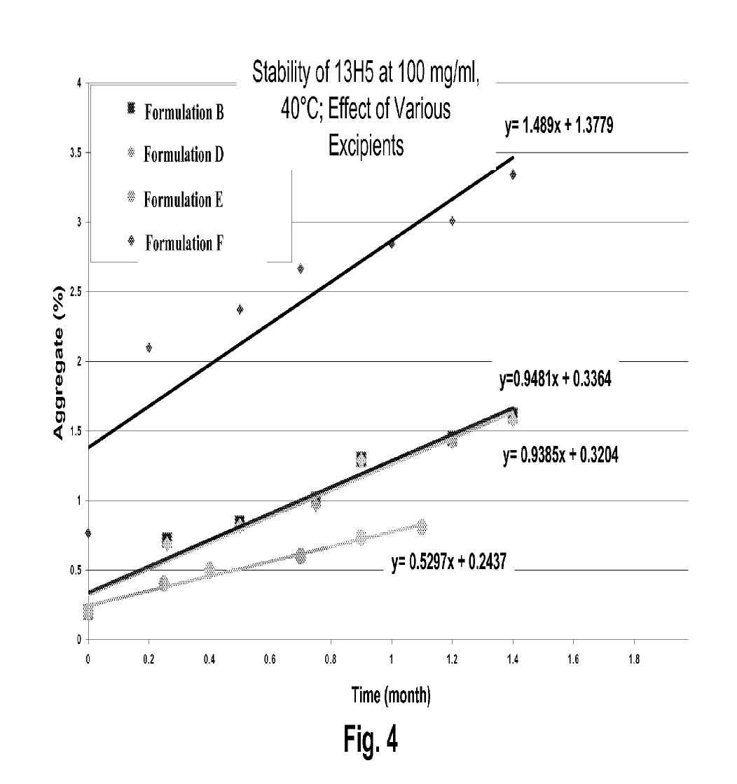

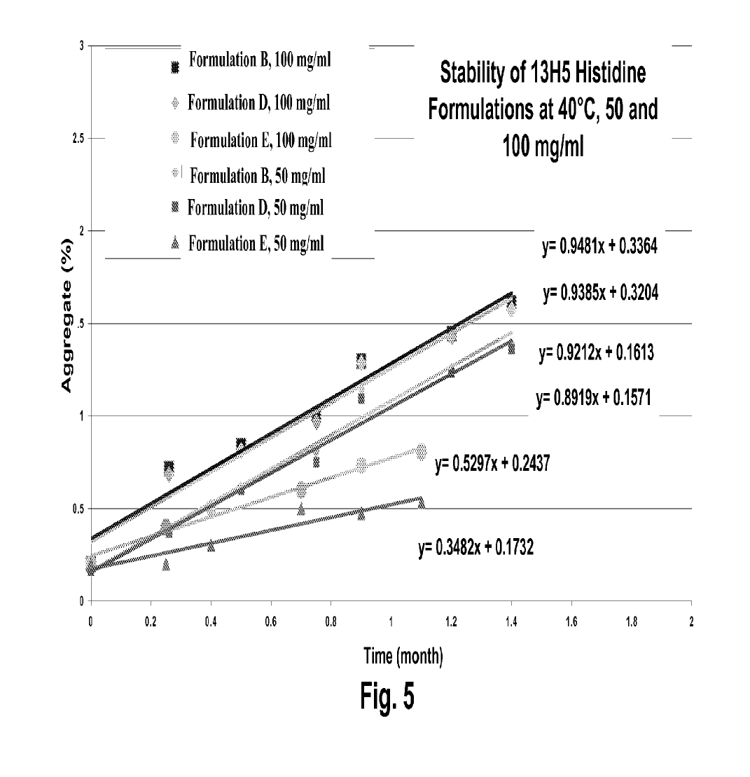

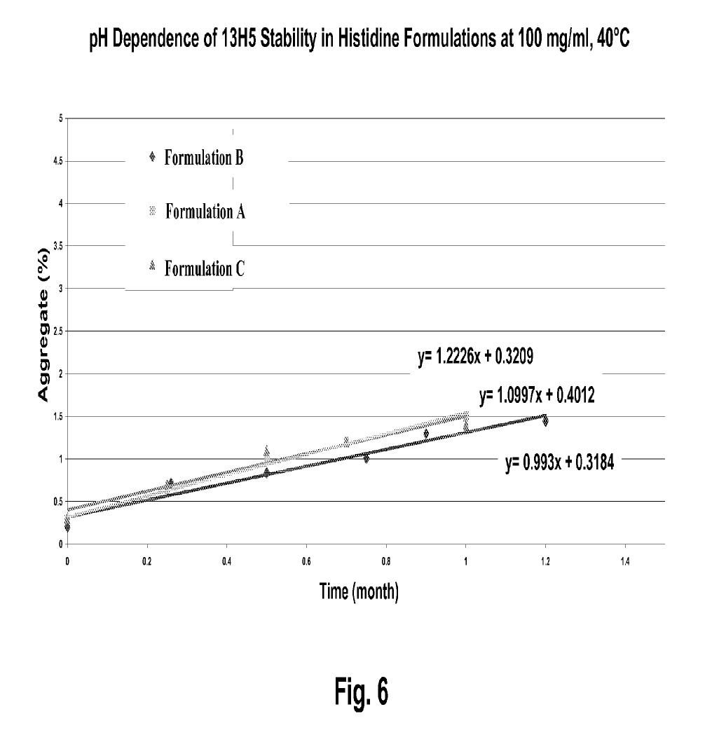

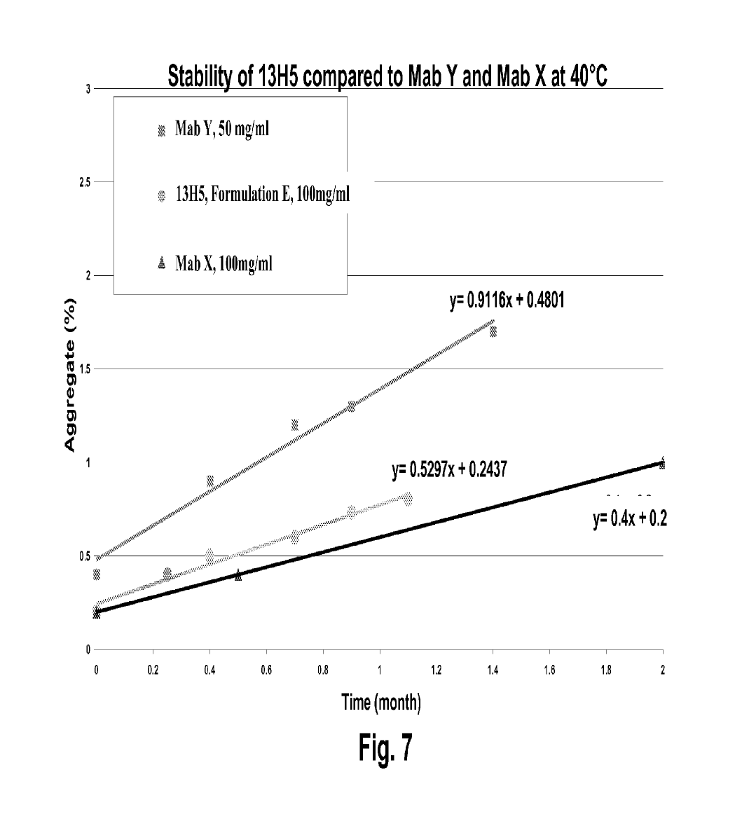

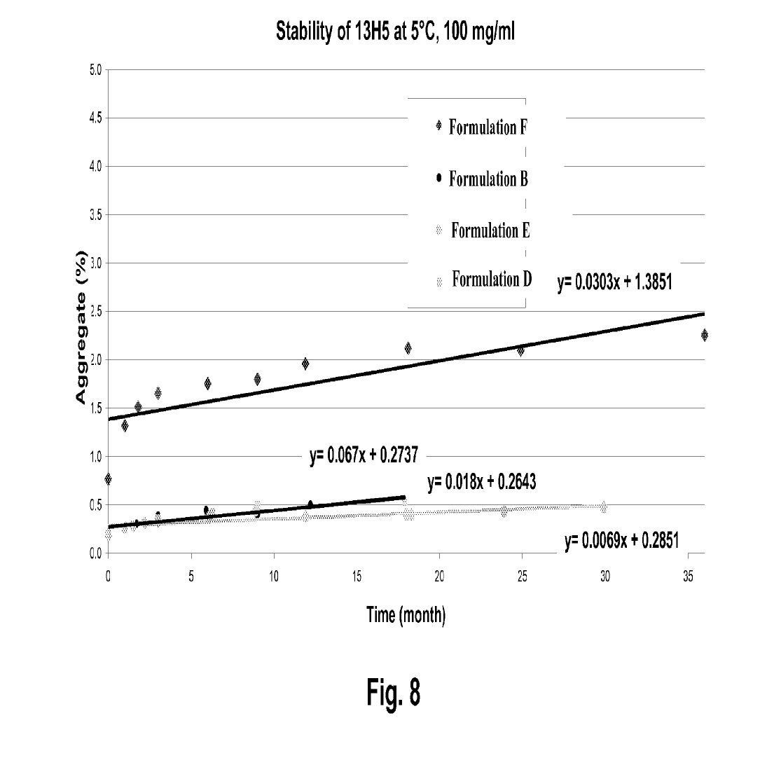

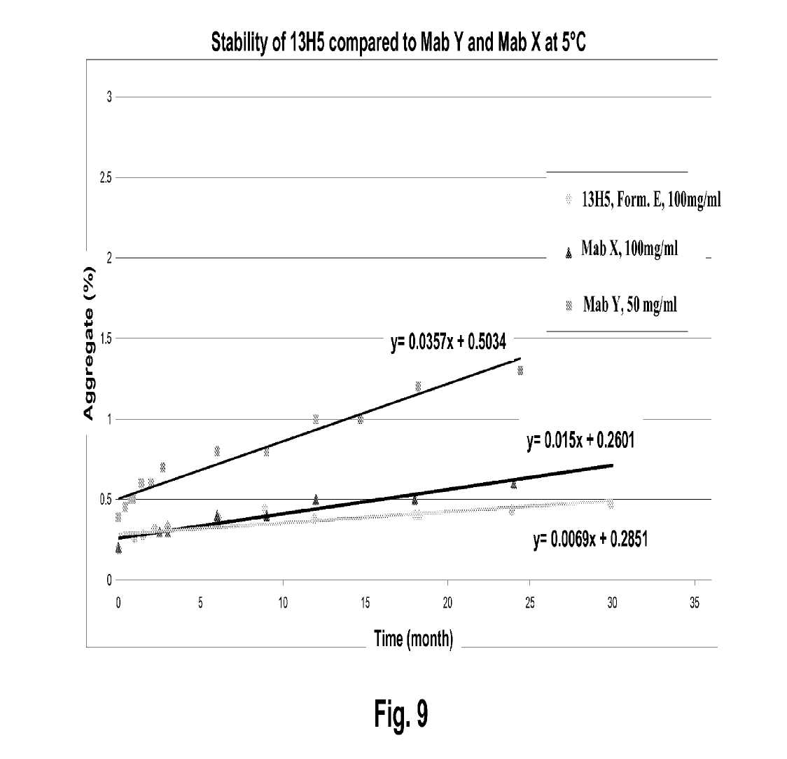

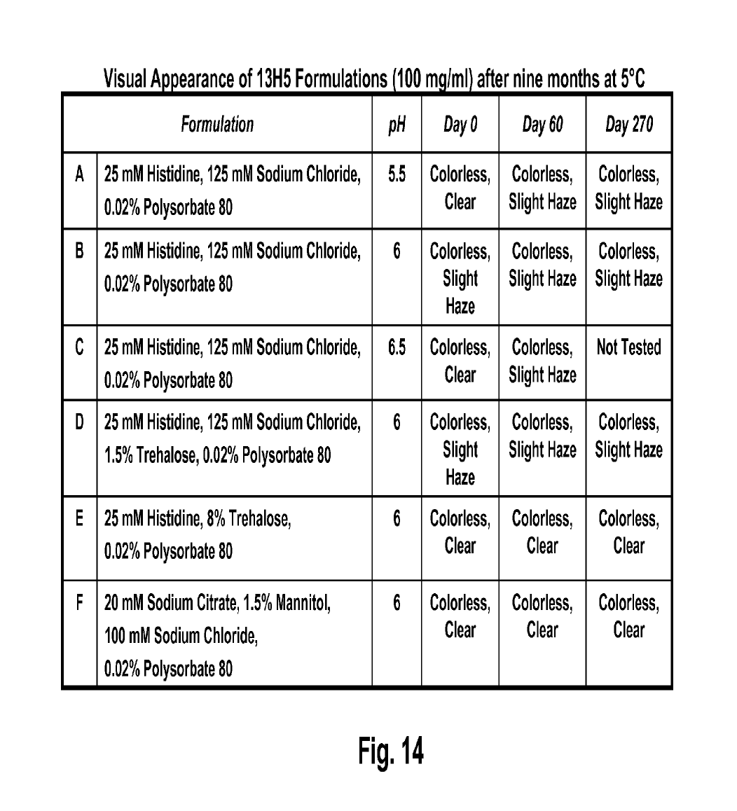

Antibody aggregate formation in various 13H5 formulations was monitored using size exclusion chromatography. Samples were stored at 5° C. or 40° C. 40° C. storage was used to simulate the effects of extended storage time. Experimental results are presented in FIGS. 2-14. FIGS. 2 and 3 show that alteration of the manufacturing process changes the aggregation profile of 13H5. FIG. 4 shows the effect of various excipients on the stability of 13H5 antibody formulations stored at 40° C.; formulation E with 8% Trehalose and 0.02% Polysorbate 80 shows the lowest aggregation rate. FIG. 5 addresses the effect of protein concentration on formulation stability. For all formulations examined, stability is lower at 100 mg/ml antibody concentration compared to that of at 50 mg/ml. Stability of 100 mg/ml 13H5 in formulation E is higher that the stability of 50 mg/ml 13H5 in formulation B or D. FIG. 6 shows that the formulation pH (within the examined range of pH 5-7) has no effect on stability. FIGS. 7 and 9 shows that the stability of 13H5 in formulation E is very similar to that of two unrelated clinical candidate high concentration liquid antibody formulations. FIG. 8 presents a stability comparison of various high concentration 13H5 formulations stored at 5° C.; formulations B, E, and D display identical stability characteristics that are better than that of formulation F.

Antibody fragmentation in various 13H5 formulations was ascertained by RP-HPLC. FIGS. 10 and 11 show the fragmentation rates of 13H5 in formulation B and E stored at 40° C. and 5° C., respectively. The observed fragmentation rates are identical in the two formulations and very similar to the fragmentation rate of an unrelated antibody in high concentration liquid formulation.

The visual appearance of 13H5 formulations after two month at 5° C. are summarized in Table 2.

TABLE 2
Visual appearance of 13H5 formulations
stored for two months at 5° C.

| Formulations | | pH | Day 0 | Day 60 |
|---|---|---|---|---|
| A | 25 mM Histidine, 125 mM Sodium | 5.5 | Colorless, | Colorless, |
| | Chloride, 0.02% Polysorbate 80 | | clear | slight haze |
| B | 25 mM Histidine, 125 mM Sodium | 6 | Colorless, | Colorless, |
| | Chloride, 0.02% Polysorbate 80 | | slight haze | slight haze |
| C | 25 mM Histidine, 125 mM Sodium | 6.5 | Colorless, | Colorless, |
| | Chloride, 0.02% Polysorbate 80 | | clear | slight haze |
| D | 25 mM Histidine, 125 mM Sodium | 6 | Colorless, | Colorless, |
| | Chloride, 1.5% Trehalose, 0.02% | | slight haze | slight haze |
| | Polysorbate 80 | | | |
| E | 25 mM Histidine, 8% Trehalose, | 6 | Colorless, | Colorless, |
| | 0.02% Polysorbate 80 | | clear | clear |
| F | 20 mM Sodium Citrate, 1.5% | 6 | Colorless, | Colorless, |
| | Mannitol, 100 mM Sodium Chloride, | | clear | clear |
| | 0.02% Polysorbate 80 | | | |

Formulation E displayed the highest stability under all conditions examined. Formulation E is colorless and clear after two month storage at 5° C. Based on its superior stability, formulation E was selected to be used as a clinical candidate high concentration 13H5 formulation. 13H5 in formulation E at a concentration of 100±5 mg/ml is hereinafter referred to as “13H5 fE” formulation.

### 6.2. Example 2

### Physical Characterization of 13H5 fE Formulation

The following section describes methods that may be used to further characterize the 13H5 fE formulation comprising 100 mg/ml 13H5 anti-human interferon alpha antibody in 25 mM Histidine (pH 6.0), 8% Trehalose, 0.02% Polysorbate 80 in a sterile aqueous solution.

### 6.2.1. Size Exclusion Chromatography (SEC)

Size exclusion chromatography may be performed to analyze the antibody formulation for the presence of antibody aggregates and fragments. The test samples are injected onto a high resolution size exclusion column (e.g., G3000 SW_{XL} 5 μm, 300 Å, 7.8×300 mm, TosoHaas). The mobile phase is 0.1 M di-sodium phosphate, 0.1 M sodium sulphate and 0.05% sodium azide (pH 6.7), running isocratically at a flow rate of 0.25-1.0 mL/min. Eluted protein may be detected by UV absorbance at 280 nm and collected for further characterization. The relative amount of any protein species detected is reported as the area percent of the product peak as compared to the total area of all other detected peaks excluding the initial excluded volume peak. Peaks eluting earlier than the antibody monomer peak are recorded in the aggregate percentile, while peaks eluting later than the antibody monomer peak, but earlier than the buffer peak, are recorded in the fragment percentile. The hydrodynamic radius and molecular weight of the individual peaks may be obtained with a coupled multiangle light scattering detector.

SEC may be used to monitor antibody aggregate formation and antibody fragmentation in a formulations stored for extended time periods (e.g., multiple measurements performed over 9 months). The formulation may be stored at different temperature ranges (e.g., 2-8° C., 20-24° C. and 38-42° C.). Temperature ranges above the proposed clinical storage temperature (2-8° C.) are used to stress the formulation with the goal of simulating the effects of storage beyond 9 months. The ratio of fragments and aggregates is expected to increase over time; this increase is likely to be accelerated at elevated temperatures. A finding that fragmentation and aggregation rates are constant within each temperature range would show that higher storage temperatures accurately simulate an accelerated time scale.

The logarithm of the estimated rates of fragmentation/aggregation (log(rate)) may also be determined. A finding that the log(rate) shows a linear dependence to the reciprocal of the storage temperature (1/T (K⁻¹) would allow the investigator to predict the rate of aggregation/fragmentation of the formulation at any temperature or, more importantly, the formulation characteristics at any time at a given temperature.

In situations where the chromatography peaks corresponding to aggregates and fragments are not be sufficiently distinct from each other, or from the monomer peak (e.g., at low relative levels of aggregates/fragments), SEC may not serve as an accurate measure of fragmentation/aggregation.

### 6.2.2. Analytical Ultracentrifugation

Analytical ultracentrifugation (AUC) may also be used to characterize the antibody formulation for the presence of antibody aggregates and fragments. AUC is an orthogonal technique which determines the sedimentation coefficients (reported in Svedberg, S) of macromolecules in a liquid sample. Like SEC, AUC is capable of separating and detecting antibody fragments/aggregates from monomers and is further able to provide information on molecular mass. Compared to SEC, AUC eliminates the possibility of aggregate loss due to solid-phase interaction and is better able to resolve differing species of a given macromolecule.

Sedimentation velocity experiments may be performed using an analytical ultracentrifuge, for example, Beckman Optima XL-A. Test samples are diluted to an antibody concentration of 0.5 mg/ml with reference buffer (e.g., 20 mM citric acid, 100 mM NaCl, 1.5% mannitol, 50 μM diethylenetriamine-pentaacetic acid, 0.02% Polysorbate 80, pH 6.0). 415 μl of the diluted antibody sample and 412 μl or the reference buffer is loaded into a 12 mm centrifuge cell in the sample and reference channels, respectively. Loaded cells are placed into an AN-50Ti analytical rotor and equilibrated to 25° C. Samples are scanned at 280 nm with a rotor speed of 42000 rpm at full vacuum. A total of 80 scans for each cell are collected for analysis. The first scan for each sample is excluded from downstream data processing to avoid artifacts caused by meniscus.

The data is analyzed using the c(s) method developed by Peter Shuck at N.I.H. and the SEDFIT (version 8.8) program with implemented c(s). Using the c(s) method, raw data scans are directly fit to a Lamm function of S in order to derive a distribution of sedimentation coefficients. The parameters used for the fitting procedure are resolution, 400; confidence interval, 0.75; grid size, 1000; partial specific volume, 0.7245; buffer density, 1.000; and buffer viscosity, 0.1002. Frictional ratio, meniscus and bottom positions are set as fitted parameters. Time independent noise is also fitted. The detected peaks are integrated and classified as follows: from 0 to 6 S, fragments; from 6 to 9 S, monomer; and from 9 to 20 S, aggregates.

AUC may be used to characterize antibody formulations with low relative levels of aggregation and fragmentation. AUC may be able to better resolve antibody fragments and aggregates from the monomer species in situations that are beyond the resolution capabilities of SEC. peaks. AUC estimates of the molecular mass of an aggregate peak may also be used as an indicator of its composition (e.g., dimers vs. higher multimers).

Compared to SEC, AUC may also able to better resolve differing species of a given macromolecule. It is, however, necessary to establish first the proper sample dilution rate, as the noise/signal ratio of AUC is dependent on the antibody concentration in the sample.

### 6.2.3. Turbidity Measurement:

Protein aggregation in the antibody formulation may also be characterized by turbidity measurement. Turbidity is a measure of the amount by which the particles in a solution scatter light and, thus, may be used as a general indicator of protein aggregation or denaturation. Elevated turbidity may indicate a higher level of aggregation or an increased number/increased size of particles.

Turbidity measurement may be performed with a turbidimeter (e.g., 2100AN or 2100N, Hatch) following the manufacturer's instructions. Approximately 3 to 4 ml of formulation sample is transferred into a glass test tube and degassed for 2 minutes using an in-line vacuum system. The degassed sample is then placed into a turbidimeter (e.g., 2100AN or 2100N, Hatch) sample compartment at room temperature for analysis. The turbidimeter is calibrated with STABLCAL® Stabilized Formazin Turbidity standard (Hatch) at 40, 200, 1000 and 4000 NTU (nephelometric turbidity unit) and verified by analyzing control suspensions of formazin at 3, 6, 18, 30 and 60 NTU.

### 6.2.4. Particle Count

The number and size of particles in a particular formulation may be determined using a particle counter (e.g., Beckman Coulter Multisizer 3) according to the manufacturers instruction.

### 6.2.5. Viscosity Profile

Viscosities of antibody formulations may be measured using a viscometer (e.g., ViscoLab 4000 Viscometer System from Cambridge Applied Systems equipped with a ViscoLab Piston (0.3055″, 1-20 cP)). The viscometer is calibrated before use with the appropriate standards (e.g., S6S Reference Standard from Koehler Instrument Company, Inc.). The viscometer is connected to a water bath to equilibrate the system to 20° C. Piston is checked using S6S viscosity reference standard (8.530 cP @20.00° C.). Piston is also checked using RODI H₂O (1.00 cP @20.0° C.). The piston is cleaned and rinsed thoroughly with soap and water between measurements of each different solution type. Subsequently the system is cooled to ≦2° C. Once the system temperature is at or below 2° C., sample is loaded into the chamber and the piston is lowered into the sample. After sample is equilibrated to the temperature of the chamber, measurement is initiated. The temperature is increased at 1° C. increments every 7-10 minutes to a final temperature of ≧25° C. The viscosity result is recorded immediately prior to increasing the temperature. The piston remains in motion during measurements to minimize the need for re-equilibration.

### 6.2.6. Differential Scanning Calorimetry

Differential Scanning Calorimetry (DSC) may be used to ascertain changes over time in the thermal stability of an antibody in a particular formulation. Thermal melting temperatures (Tₘ) are determined with a differential scanning calorimeter (e.g., VP-DSC from MicroCal, LLC) following the manufacturer's instruction. In one example, VP-DSC is used at a scan rate of 1.0° C./min and with a temperature range of 25-120° C. A filter period of 8 seconds is used along with a 5 minute pre-scan thermostating. Samples are prepared by dialysis into 10 mM Histidine-HCl, pH 6 using Pierce dialysis cups (3.5 kD). Average Mab concentrations are 50 μg/mL as determined by A₂₈₀. Melting temperatures are determined following the manufacturer's instructions using software supplied with the system.

### 6.3. Biochemical Characterization of the 13H5 fE Formulation

### 6.3.1. Liquid Chromatography Mass Spectrometry (LC-MS)

Liquid Chromatography Mass Spectrometry (LC-MS) may be used to characterize a degradation fragment detected by SEC or AUC in the antibody formulation.

Peak SEC column fractions containing the degradation fragment are collected and digested with N-Glycosidase F, also known as PNGase F, at 37° C. overnight. PNGase F is an amidase used to deglycosylate protein samples. The enzyme cleaves between the innermost GlcNAc and asparagine residues of high mannose, hybrid and complex oligosaccharides on N-linked glycoproteins. The deglycosylated samples mixed with a reducing buffer (e.g., 2.5 mg/mL DTT, 6.0 M guanindine HCl, pH 8.2) and kept at 56° C. in a water bath for 60 minutes. Neat 4-vinylpyridine (e.g., Aldrich Chem. Co., WI) is then added to the sample, and the reaction mixture is held at ambient temperature for 30 minutes. The deglycosylated, reduced and alkylated sample is immediately loaded onto a reversed phase column in order to separate the modified samples from the reactants.

Deglycosylated, reduced, and alkylated samples are fractionated using a reversed phase column (e.g., Jupiter 5 μm C4, 300 Å, 250×2.00 mm, Phenomenex) with a binary gradient HPLC system (Agilent 1100). Mobile phase A consists of 30% acetonitrile in water with 0.1% trifluoroacetic acid and mobile phase B consists of 50% acetonitrile in water with 0.1% trifluoroacetic acid. The samples are separated using a linear gradient of 30-50% acetonitrile in water, over 16 min. with a flow rate of approximately 200 μL/min. The column effluent is directed to a UV detector and then split 1:1, one half going through a switching valve on an Ion Trap mass spectrometer (e.g., LTQ, ThermoElectro, San Jose, Calif.), and the remaining half to waste.

The ion-trap mass spectrometer is calibrated before the experimental run using a mixture of caffeine, L-methionyl-arginyl-phenylalanyl-alanine acetate H₂O, and Ultramark 162. The Electrospray Ionisation Mass Spectrometry (ESI-MS) data is acquired in positive ESI full scan mode. The BioWork deconvolution program (ThermoFinnigan) may be used to reconstruct the mass spectra and obtain the molecular masses of the peptides/proteins from their original mass spectra. The mass data subsequently is used to determine the identity of the degradation fragment.

### 6.3.2. Differential Scanning Calorimetry

Differential Scanning Calorimetry (DSC) may be used to ascertain changes over time in the thermal stability of an antibody in a particular formulation. Thermal melting temperatures (Tₘ) are determined with a differential scanning calorimeter (e.g., VP-DSC from MicroCal, LLC) following the manufacturer's instruction. In one example, VP-DSC is used at a scan rate of 1.0° C./min and with a temperature range of 25-120° C. A filter period of 8 seconds is used along with a 5 minute pre-scan thermostating. Samples are prepared by dialysis into 10 mM Histidine-HCl, pH 6 using Pierce dialysis cups (3.5 kD). Average Mab concentrations are 50 μg/mL as determined by A₂₈₀. Melting temperatures are determined following the manufacturer's instructions using software supplied with the system.

### 6.3.3. Isoelectric Focusing Gel Electrophoresis

Isoelectric point measurements of 13H5 may be used to ascertain the antibody's chemical stability in a given formulation. Isoelectric points are determined using a Pharmacia Biotech Multiphor 2 electrophoresis system with a multi temp 3 refrigerated bath recirculation unit and an EPS 3501 XL power supply. Pre-cast ampholine gels (Amersham Biosciences, pI range 2.5-10) are loaded with 5 μg of protein. Broad range pI marker standards (Amersham, pI range 3-10, 8 μL) are used to determine relative pI for the Mabs. Electrophoresis is performed at 1500 V, 50 mA for 105 minutes. The gel is fixed using a Sigma fixing solution (5×) diluted with purified water to 1×. Staining is performed overnight at room temperature using Simply Blue stain (Invitrogen). Destaining is carried out with a solution of 25% ethanol, 8% acetic acid and 67% purified water. Isoelectric points are determined using a Bio-Rad Densitometer relative to calibration curves of the standards.

### 6.3.4. Disulfide Bond Determination

Disulfude bond determination protocols may be used to monitor the stability of disulfide bridge crosslinks in a particular antibody formulation. Antibody samples are denatured, for example, in 10 mM phosphate buffer, 250 mM NaCl, 5 mM NEM, 6 M Guanidine, pH 7.0 at 37° C. for 1 to 3 hr. The denatured samples are diluted 6 fold with 100 mM phosphate buffer, 0.1 mM EDTA, pH 7.0, to which Endoproteinase Lys-C (e.g., Roche) is added at a 1:10 enzyme to protein ratio. The reaction mixtures are incubated at 37° C. for 16 to 24 hours. In half of the reaction mixture disulfide bridges are reduced by adding 5-10 μL of 100 mM DTT followed by incubation at 37° C. for 1 hr. Lys-C digested samples are fractionated by reverse-phase HPLC (e.g., Phenomenex Jupiter 5m C18 column; 250×2.1 mm). Eluant is analyzed by an UV-detector and an in-line LCQ or LTQ Ion Trap mass spectrometer (e.g., ThermoElectron). The RP-HPLC mobile phase A is 0.1% TFA in H₂O and mobile phase B is 0.1% TFA in acetonitrile. The peptides are eluted at a flow rate of 0.2 mL/min with the following step gradient: 1) 0-2 min, 5% Mobile Phase B; 2) 2-32 min, 5-20% Mobile Phase B; 3) 32-132 min, 20-40% Mobile Phase B; 4) 132-152 min, 40-60% Mobile Phase B; 5) 152-155 min, 60-95% Mobile Phase B.

The ion-trap mass spectrometer is calibrated before the experimental run using a mixture of caffeine, L-methionyl-arginyl-phenylalanyl-alanine acetate H₂O, and Ultramark 162. The Electrospray Ionisation Mass Spectrometry (ESI-MS) data is acquired in positive ESI full scan mode. The BioWork deconvolution program (ThermoFinnigan) may be used to reconstruct the mass spectra and obtain the molecular masses of the peptides from their original mass spectra. Comparison of the mass data acquired using the DTT reduced and non-reduced samples allows the identification of the disulfide crosslinked peptides.

### 6.3.5. Binding Affinity Characterization

Binding affinity of 13H5 monoclonal antibody recovered form 13H5 fE formulation may be determined by surface plasmon resonance (see, e.g., Jonsson et al., Biotechniques 11(5):620-627 (1991); Johne, B., Molecular Biotechnology 9(1):65-71(1989)) using a BIAcore 3000 instrument (BIAcore, Inc., Piscataway, N.J.). 13H5 antibody is captured on a Prot-G coated CM5 chip. A Prot-G coated CM5 chip with captured isotype control human-IgG (Sigmna) antibody is used for reference purposes. Various human interferon alpha isotypes may be used as a binding partner for 13H5 (see, US Patent Application No. 2007/0014724A1). Interferon alpha dissolved in HBS-EP running buffer is passed over the chip at a rate of 25 ul/min. 5 minutes of association time is followed by a 10 minute dissociation period. Independent measurements are performed by exposing the chips to different concentrations of interferon alpha (e.g. concentrations between 10 nM and 80 nM). Chips are regenerated by a 0.4 minute wash with 20 mM NaOH+400 mM NaCl at a flow rate of 100 ul/min. Once the entire data set is collected, the resulting binding curves are globally fitted to a 1:1 Langmuir binding model using BIAevaluation software (BIAcore, Inc., Piscataway, N.J.). This algorithm calculates both the association rate (kₒₙ) and the dissociation rate (k_{off}), from which the apparent equilibrium binding constant, K_{D}, is deduced as the ratio of the two rate constants, k_{off}/kₒₙ. A more detailed explanation of how the individual rate constants are derived can be found in the BIAevaluation Software Handbook (BIAcore, Inc., Piscataway, N.J.).

### 6.4. Characterization of Ultra High Concentration Liquid Formulations of the 13H5 Antibody

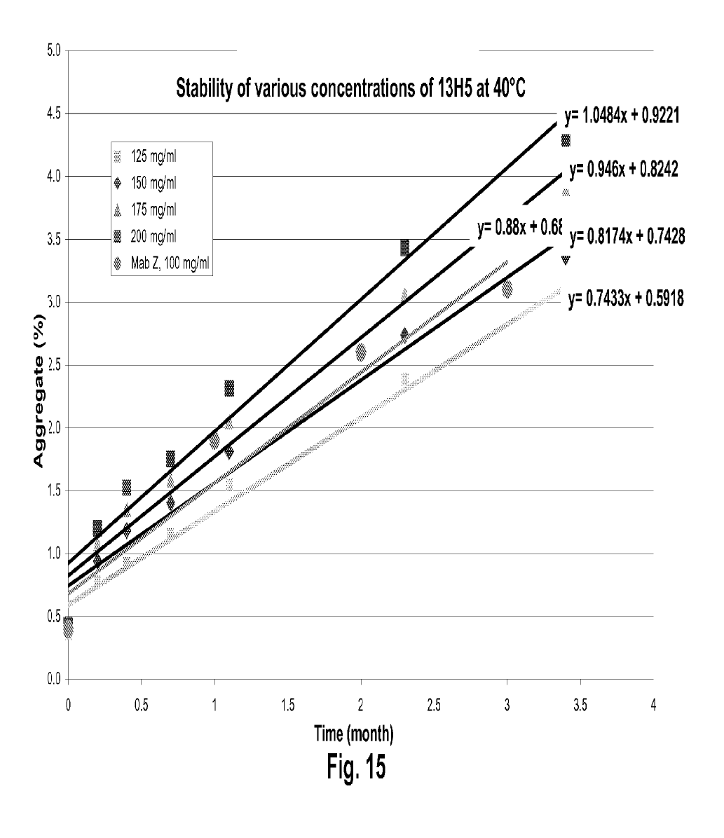

The stability of liquid formulations comprising 125 mg/ml, 150 mg/ml, 175 mg/ml and 200 mg/ml 13H5 antibody were ascertained using the experimental methods described above. The base formulation contained 25 mM histidine-HCl (pH 6.0), 5% sucrose, and 0.02% Polysorbate 80. The stability measurement results for the ultra high concentration antibody formulations are presented in FIGS. 15-18.

Preparation of ultra concentrated liquid formulations: Purified 13H5 antibody was nanofiltered using a Planova 20N filter to remove particulate matter. 13H5 formulations were prepared using Tangential Flow Filtration (TFF). The nanofiltered 13H5 antibody was concentrated to approximately 25 mg/ml on a Millipore Labscale TFF device. The antibody was then 5× diafiltered into 25 mM histidine-HCl (pH 6.0), 5% sucrose. Once the buffer exchange was complete, the antibody was concentrated to a concentration slightly higher than the final target concentration. Polysorbate 80 was introduced by spiking (1:100 dilution) the concentrated antibody preparation with a concentrated stock solution of 25 mM histidine-HCl (ph 6.0), 5% sucrose, 2% Polysorbate 80. The final concentration of 13H5 was adjusted to the target concentration with the final formulation buffer (e.g., 25 mM histidine-HCl (pH 6.0), 5% sucrose, 0.02% Polysorbate 80).

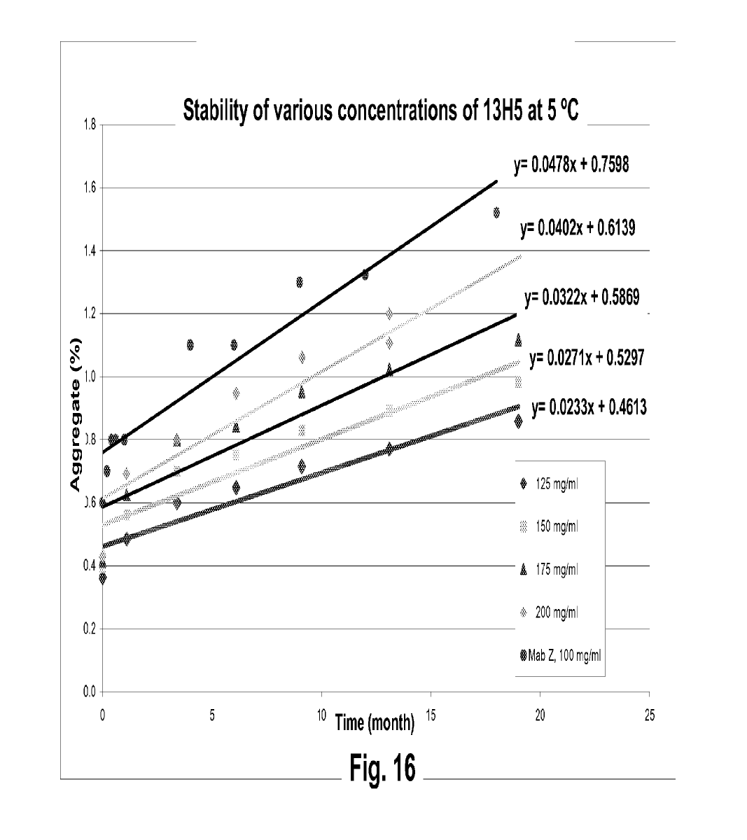

Antibody aggregate formation by the ultra concentrated liquid 13H5 formulations was monitored using size exclusion chromatography. Samples were stored at 5° C. or 40° C. 40° C. storage was used to simulate the effects of extended storage time. Experimental results obtained at 40° C. and 5° C. are presented in FIGS. 15 and 16, respectively. The rate of 13H5 aggregate formation slightly increased with increased antibody concentration. The aggregation properties of the ultra concentrated 13H5 antibody formulation was comparable to the aggregation properties of a reference antibody formulation containing 100 mg/ml antibody Z.

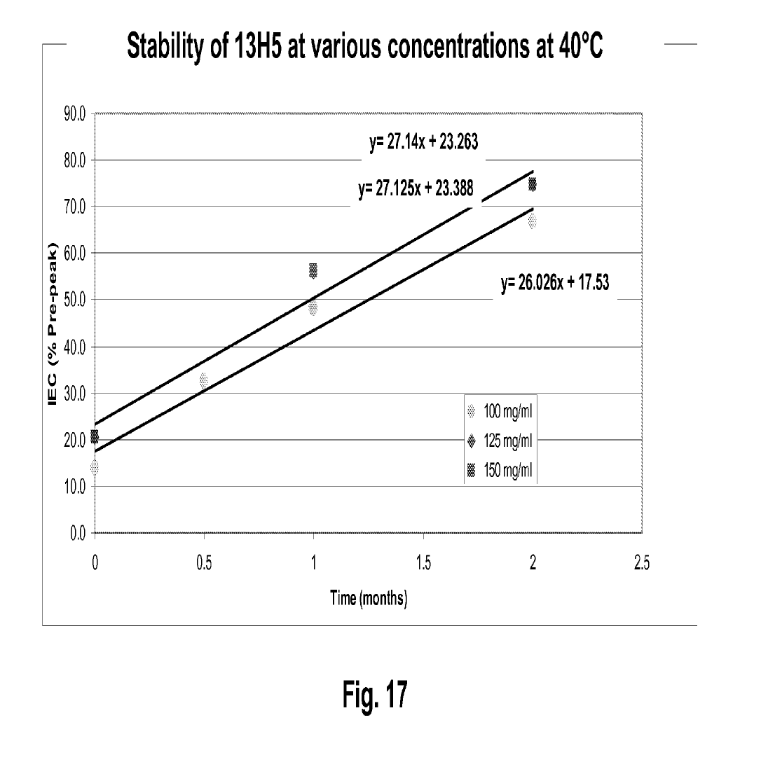

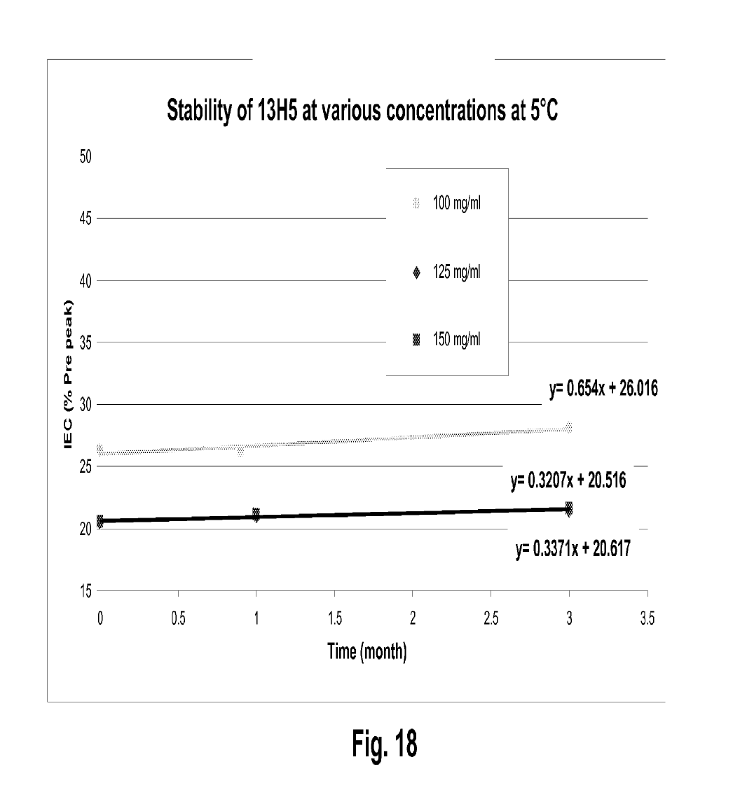

Antibody degradation in ultra concentrated liquid 13H5 formulations was monitored using ion exchange chromatography. Samples were stored at 5° C. or 40° C. 40° C. storage was used to simulate the effects of extended storage time. Experimental results obtained at 40° C. and 5° C. are presented in FIGS. 17 and 18, respectively. The rate of 13H5 degradation was unaffected by increased antibody concentration.

### 6.5. Formulation Stability in Pre-Filled Syringes

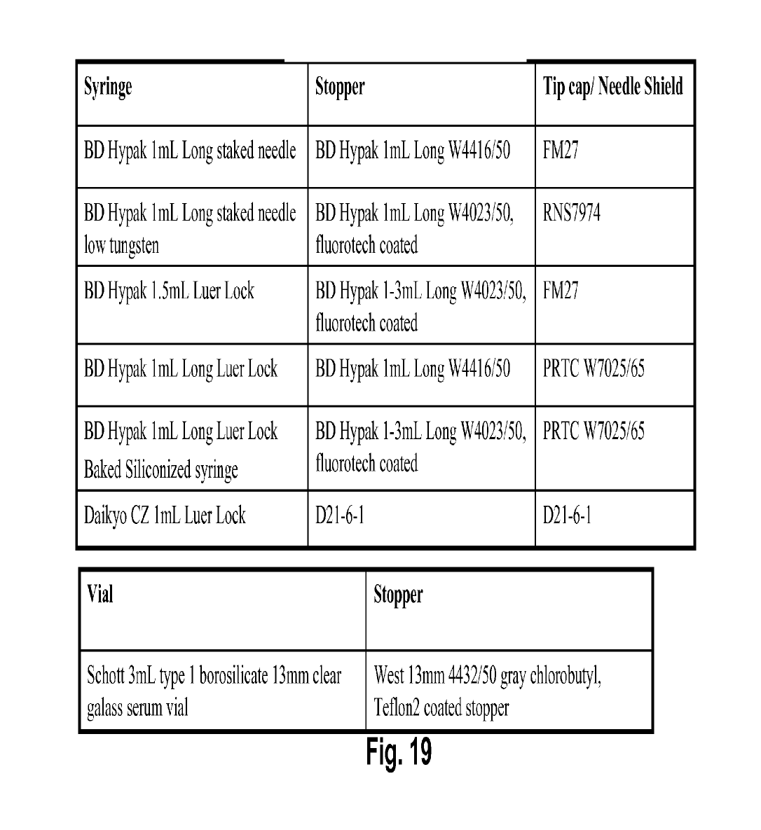

The stability of a formulation comprising 100 mg/ml 13H5 antibody, 25 mM histidine, 8% trehalose and 0.02% PS-80 at pH 6.0 was tested in pre-filled syringes. A description of the various types of syringes tested is provided in FIG. 19. Stability testing was performed by loading 1 ml of the formulation into a syringe and storing the formulation-filled syringe at 5° C., 25° C. or 40° C. for extended periods of time. Formulation stability was analysed using the analytical methods described herein. Particle formation was assessed by visual inspection. Protein aggregation was measured by subjecting the formulation recovered from the syringe to SEC. Deamidation profile of the 13H5 antibody recovered from the syringes at various time points was determined by IEC.

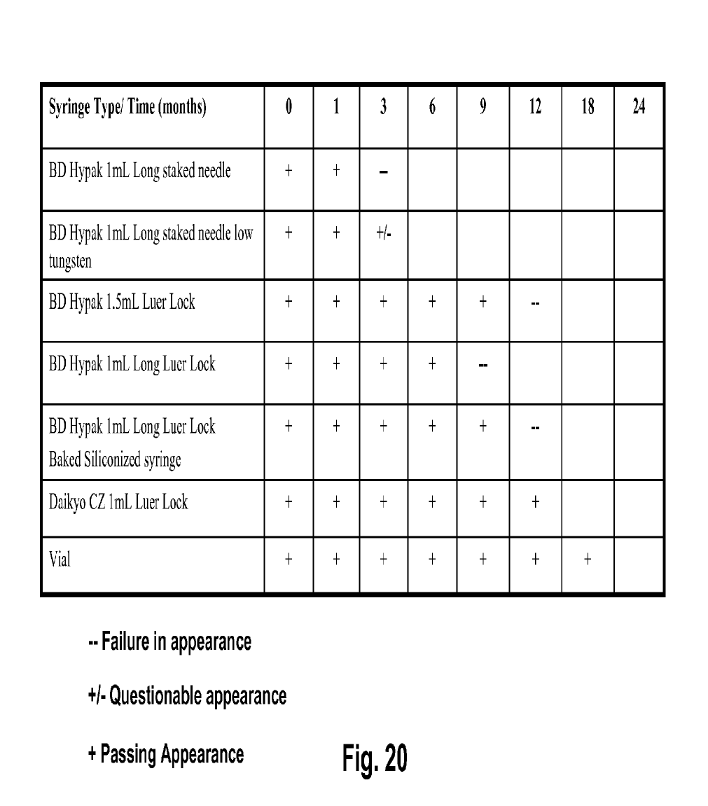

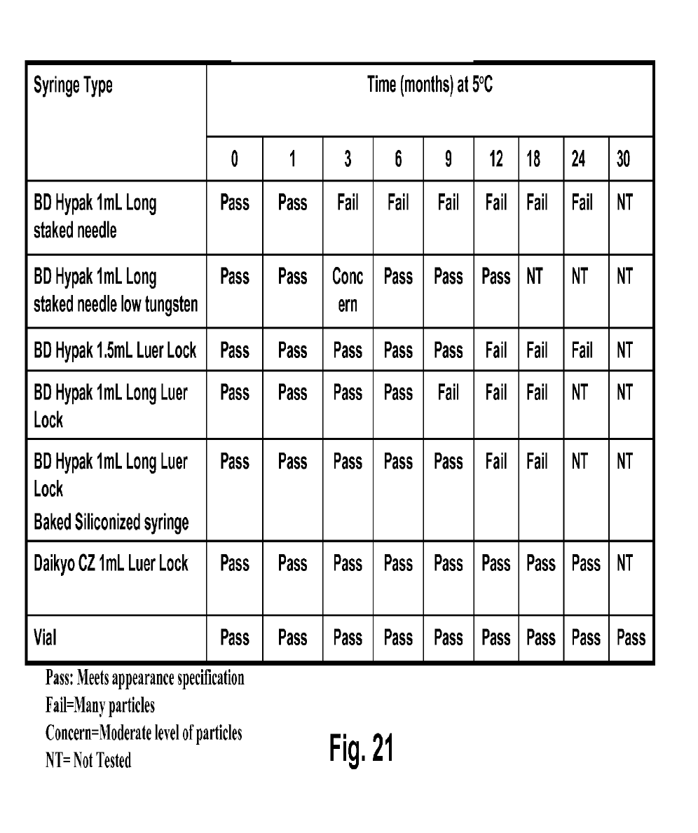

Particle formation as determined by visual inspection is a key determinant of formulation stability in pre-filled syringes. Particle formation detected in various pre-filled syringes stored at 5° C. is shown in FIGS. 20 and 21. After 12 month storage at 5° C., particles detectable with the naked eye appeared in all syringes tested except for the Daikyo CZ1 mL Luer Lock syringe (CZ syringe). No particle formation was detected in the CZ syringe after 12 month storage at 5° C. The stability of the formulation, as characterized by particle formation, is substantially the same in the CZ syringe as in a glass vial.

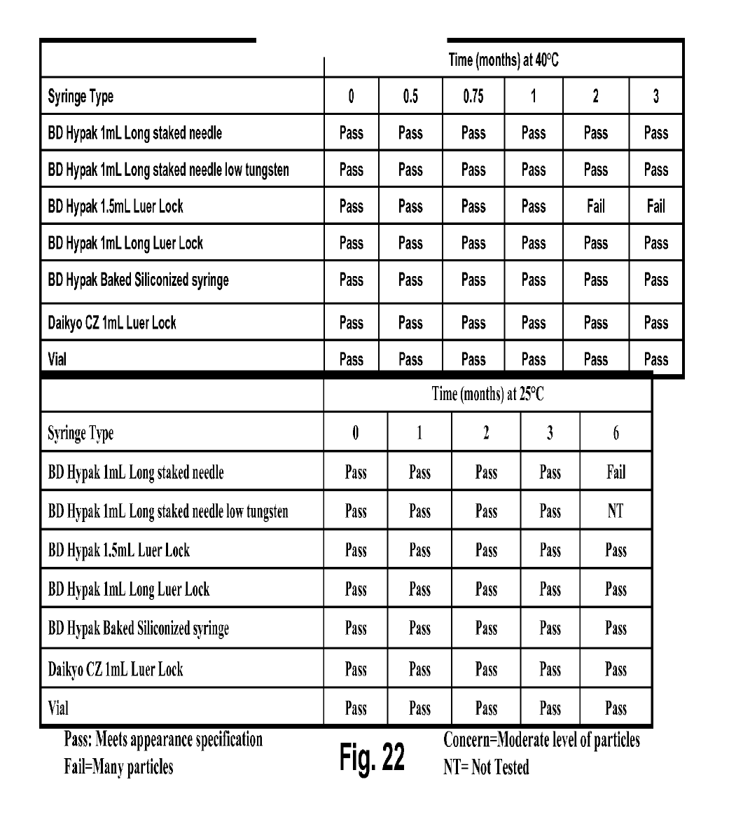

Particle formation in pre-filled syringes stored at 25 or 40° C. was also examined. Results from the experiment are shown in FIG. 22. Particle formation rates at 25 or 40° C. were not sufficiently different in the various syringes to predict which syringe has the best long term stability at 5° C.

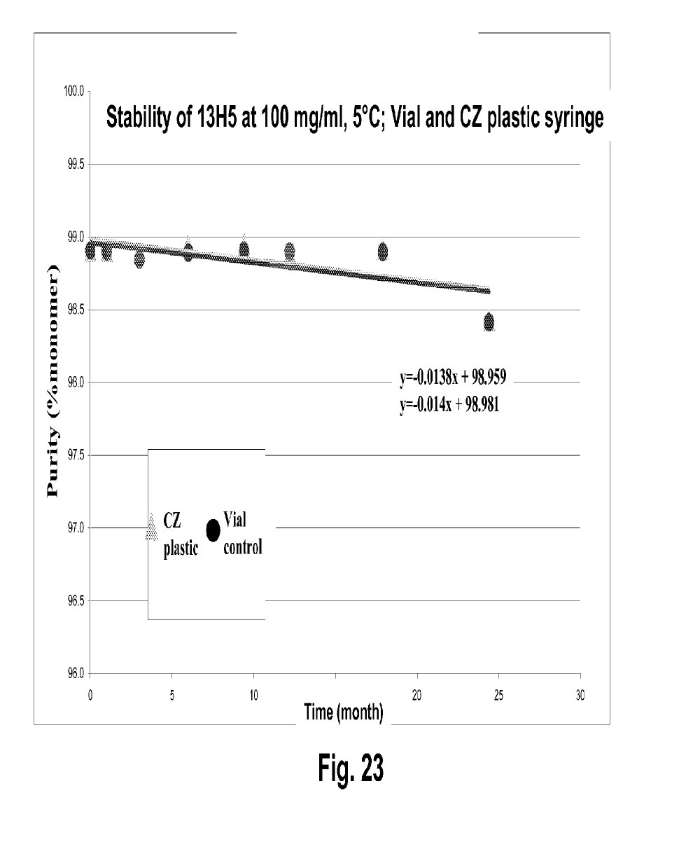

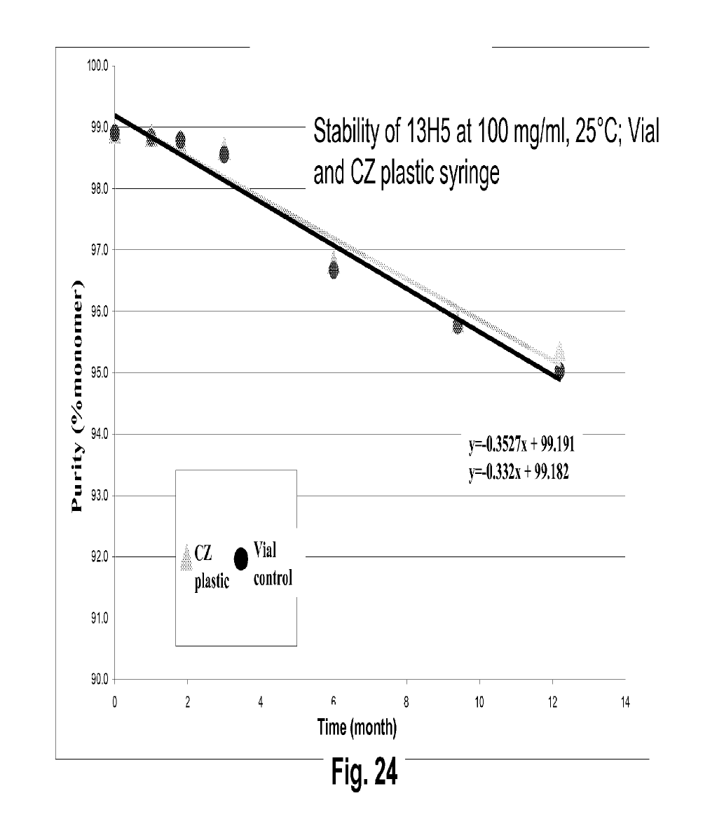

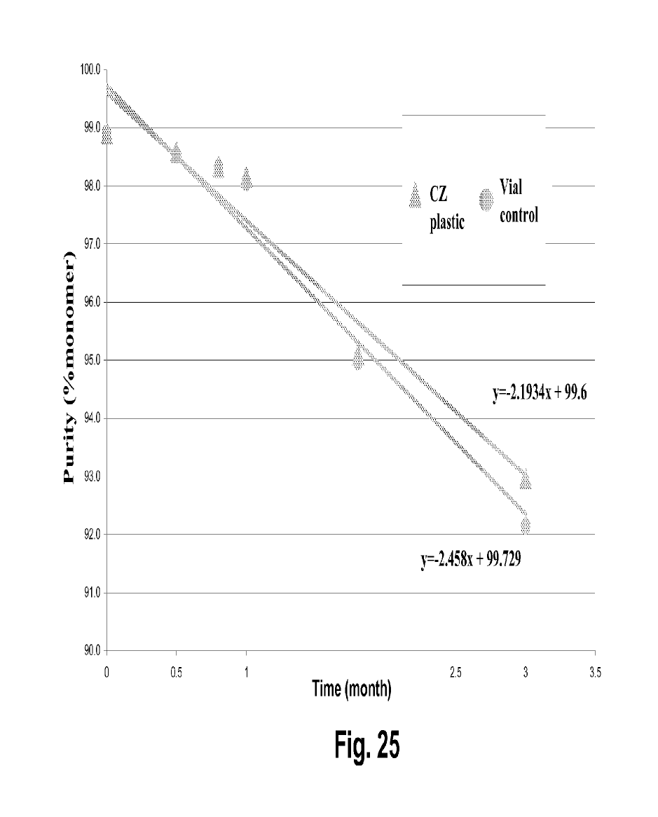

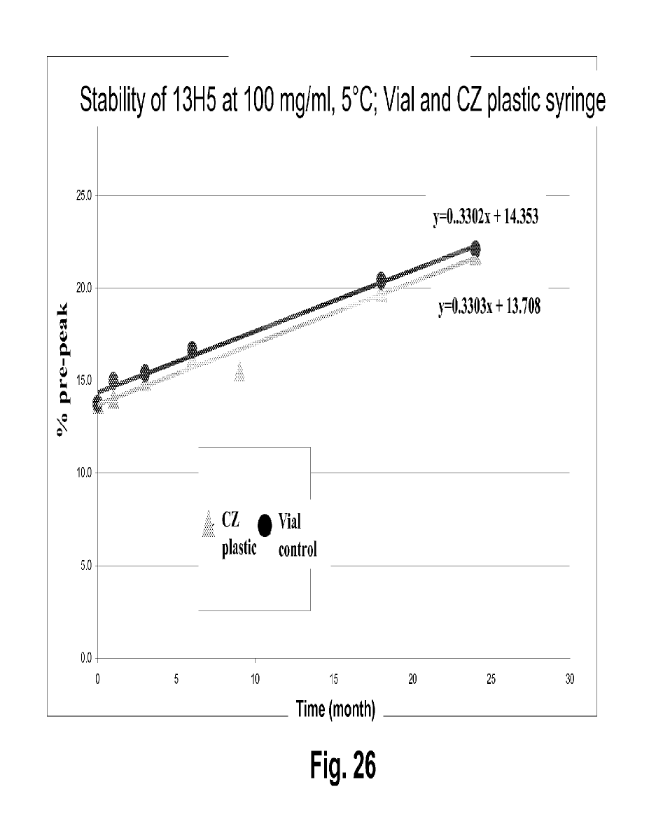

Formulation stability in a prefilled CZ syringe was further characterized using SEC and IEC. Results are shown in FIGS. 23-26. Aggregate formation rates in pre-filled CZ syringe and glass vial were substantially the same upon long term storage at all temperatures (5° C., 25° C. and 40° C.) examined. Deamidation rate of the 13H5 antibody stored at 5° C. was substantially the same in a pre-filled CZ syringe and a glass vial.

Whereas, particular embodiments of the invention have been described above for purposes of description, it will be appreciated by those skilled in the art that numerous variations of the details may be made without departing from the invention as described in the appended claims.

All publications, patents and patent applications mentioned in this specification are herein incorporated by reference into the specification to the same extent as if each individual publication, patent or patent application was specifically and individually indicated to be incorporated herein by reference. In addition, PCT Publication WO/2008/121616, filed Mar. 25, 2008, PCT Publication WO 2008/121615 filed Mar. 25, 2008, U.S. Provisional Application 61/104,174, filed Oct. 9, 2008 and U.S. Provisional Application 61/106,281, filed Oct. 17, 2008 are hereby incorporated by reference in their entirety for all purposes.

1. A pre-filled syringe containing a sterile, stable aqueous formulation comprising a 13H5 anti-human interferon alpha antibody, histidine, trehalose and polysorbate 80.

2. The pre-filled syringe of claim 1, wherein the pre-filled syringe is made of materials that are substantially free from tungsten.

3. The pre-filled syringe of claim 1, wherein the pre-filled syringe is substantially free from silicone.

4. The pre-filled syringe of claim 1, wherein said formulation is isotonic.

5. The pre-filled syringe of claim 4, wherein said formulation is suitable for subcutaneous administration or intramuscular administration.

6. The pre-filled syringe of claim 1, wherein said formulation comprises at least 50 mg/ml, at least 60 mg/ml, at least 70 mg/ml, at least 80 mg/ml, at least 90 mg/ml, at least 100 mg/ml, at least 120 mg/ml, at least 150 mg/ml, at least 160 mg/ml, at least 180 mg/ml, at least 200 mg/ml, at least 250 mg/ml, or at least 300 mg/ml of a 13H5 anti-human interferon alpha antibody.

7. The pre-filled syringe of claim 1, wherein said formulation comprises between about 50 mg/ml and about 150 mg/ml of a 13H5 anti-human interferon alpha antibody, between about 1 mM and about 100 mM histidine, between about 1% and about 40% trehalose and between about 0.001% and about 5% polysorbate 80 and wherein the pH of said formulation is between about 5 and about 7.

8. The pre-filled syringe of claim 1, wherein said formulation comprises between about 80 mg/ml and about 120 mg/ml of a 13H5 anti-human interferon alpha antibody, between about 10 mM and about 50 mM histidine, between about 4% and about 20% trehalose and between about 0.005% and about 1% polysorbate 80 and wherein the pH of said formulation is between about 5.5 and about 6.5.

9. The pre-filled syringe of claim 1, wherein said formulation comprises about 100 mg/ml of a 13H5 anti-human interferon alpha antibody, about 25 mM histidine, about 8% trehalose and between about 0.005% and about 1% polysorbate 80 and wherein the pH of said formulation is about 6.

10. The pre-filled syringe of claim 1, wherein said formulation comprises about 100 mg/ml of a 13H5 anti-human interferon alpha antibody, about 25 mM histidine, about 8% trehalose and about 0.02% polysorbate 80 and wherein the pH of said formulation is about 6.

11. The pre-filled syringe of claim 9, wherein said formulation is stable upon storage at about 5° C. for at least 6 months.

12. The pre-filled syringe of claim 9, wherein said formulation is stable upon storage at about 5° C. for at least 12 months.

13. The pre-filled syringe of claim 9, wherein said antibody or fragment thereof retains at least 90% of binding ability to a human interferon alpha polypeptide compared to a reference antibody preparation.

14. The pre-filled syringe of claim 9, wherein less than 2% of said antibody or fragment thereof forms an aggregate upon storage at about 5° C. for at least 6 months as determined by HPSEC.

15. The pre-filled syringe of claim 9, wherein less than 5% of said antibody or fragment thereof is fragmented upon storage at about 5° C. for at least 6 months as determined by RP-HPLC.

16. The pre-filled syringe of claim 9, wherein less than 30% of said antibody or fragment thereof is subject to deamidation upon storage at about 5° C. for at least 3 months as determined by IEC.

17. The pre-filled syringe of claim 9, wherein less than 60% of said antibody or fragment thereof is subject to deamidation upon storage at about 5° C. for at least 12 months as determined by IEC.

18. The pre-filled syringe of claim 9, wherein said formulation is clear and colorless upon storage at about 5° C. for at least 6 months as determined by visual inspection.

19. The pre-filled syringe of claim 9, wherein said formulation is substantially free from particulates upon storage at about 5° C. for at least 6 months as determined by visual inspection.

20-21. (canceled)


SEQUENCE LISTING (26 sequences)

SEQ ID NO: 1 - protein, 446 aa, Homo sapiens
QVQLVQSGAEVKKPGASVKVSCKASGYTFTSYSISWVRQAPGQGLEWMGWISVYNGNTNYAQKFQGRVTMTTDTSTSTAYLELRSLRSDDTAVYYCARDPIAAGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - protein, 116 aa, Homo sapiens [sequence omitted]

SEQ ID NO: 3 - protein, 5 aa, Homo sapiens
SYSIS

SEQ ID NO: 4 - protein, 17 aa, Homo sapiens
WISVYNGNTNYAQKFQG

SEQ ID NO: 5 - protein, 7 aa, Homo sapiens
DPIAAGY

SEQ ID NO: 6 - protein, 215 aa, Homo sapiens
EIVLTQSPGTLSLSPGERATLSCRASQSVSSTYLAWYQQKPGQAPRLLIYGASSRATGIPDRFSGSGSGTDFTLTISRLEPEDFAVYYCQQYGSSPRTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 7 - protein, 108 aa, Homo sapiens [sequence omitted]

SEQ ID NO: 8 - protein, 12 aa, Homo sapiens
RASQSVSSTYLA

SEQ ID NO: 9 - protein, 7 aa, Homo sapiens
GASSRAT

SEQ ID NO: 10 - protein, 9 aa, Homo sapiens
QQYGSSPRT

SEQ ID NO: 11 - protein, 121 aa, Homo sapiens
QVQLQESGPGLMKPSETLSLTCTVSGGSVSSGSYYWSWIRQPPGMGLEWIGYIYSGGGANYNPSLKSRVTISVDTSKNQFSLKLNSVTAADTAVYFCARGIPMVRGILHYWGQGTLVTVSS

SEQ ID NO: 12 - protein, 7 aa, Homo sapiens
SGSYYWS

SEQ ID NO: 13 - protein, 16 aa, Homo sapiens
YIYSGGGANYNPSLKS

SEQ ID NO: 14 - protein, 11 aa, Homo sapiens
GIPMVRGILHY

SEQ ID NO: 15 - protein, 108 aa, Homo sapiens
EIVLTQSPGTLSLSPGERATLSCRASQSVSSSFLAWYQQKPGQAPRLLIYGASSRATGIPDRFSGSGSGTDFTLTISRLEPEDFAVYYCQQYGSSPYTFGQGTKLEIK

SEQ ID NO: 16 - protein, 12 aa, Homo sapiens
RASQSVSSSFLA

SEQ ID NO: 17 - protein, 7 aa, Homo sapiens
GASSRAT

SEQ ID NO: 18 - protein, 9 aa, Homo sapiens
QQYGSSPYT

SEQ ID NO: 19 - protein, 116 aa, Homo sapiens
QVQLVQSGAEVKKPGASVKVSCKASGYTFSSYGISWVRQAPGQGLEWMGWISAYNGNTNYLQKLQGRVTLTTDTSTNTAYMELRSLRSDDTAVYYCTRDPIAAGYWGQGTLVTVSS

SEQ ID NO: 20 - protein, 5 aa, Homo sapiens
SYGIS

SEQ ID NO: 21 - protein, 17 aa, Homo sapiens
WISAYNGNTNYLQKLQG

SEQ ID NO: 22 - protein, 7 aa, Homo sapiens
DPIAAGY

SEQ ID NO: 23 - protein, 108 aa, Homo sapiens [sequence omitted]

SEQ ID NO: 24 - protein, 12 aa, Homo sapiens
RASQSVSSTYLA

SEQ ID NO: 25 - protein, 7 aa, Homo sapiens
GASSRAT

SEQ ID NO: 26 - protein, 9 aa, Homo sapiens
QQYGSSPRT